SEQUENCE LISTING (16 sequences)

SEQ ID NO: 1 - DNA, 87 bp, Artificial Sequence
  Oligonucleotide comprising the “F48” stabilization strand of a synthetic antibody.
  misc_feature 23, 24, 25, 26, 27, 28, 29, 30, 31, 32, 33, 34, 35, 36,37, 38, 39, 40, 41, 42, 43, 44, 45, 46, 47, 48, 49, 50, 51,52, 53, 54, 55, 56, 57, 58, 59, 60, 61, 62: n = any nucleotide or modified nucleotide
gtaaaacgacggccagtgtctcnnnnnnnnnnnnnnnnnnnnnnnnnnnnnnnnnnnnnnnnagattcctgtgtgaaattgttatcc

SEQ ID NO: 2 - DNA, 48 bp, Artificial Sequence
  Oligonucleotide comprising the “F22-40-25” specificity strand of a synthetic antibody.
ggataacaatttcacacaggagctctcgaggactggccgtcgttttac

SEQ ID NO: 3 - DNA, 24 bp, Artificial Sequence
  Primer B21-40
ggataacaatttcacacaggaatc

SEQ ID NO: 4 - DNA, 22 bp, Artificial Sequence
  Primer F17-50
gtaaaacgacggccagtgtctc

SEQ ID NO: 5 - DNA, 78 bp, Artificial Sequence
  Oligonucleotide comprising 78nt specificity strand of a synthetic antibody
  misc_feature 18, 19, 20, 21, 22, 23, 24, 25, 26, 27, 28, 29, 30, 31,32, 33, 34, 35, 36, 37, 38, 39, 40, 41, 42, 43, 44, 45, 46,47, 48, 49, 50, 51, 52, 53, 54, 55, 56, 57: n = any nucleotide or modified nucleotide
gtaaaacgacggccagtnnnnnnnnnnnnnnnnnnnnnnnnnnnnnnnnnnnnnnnntcctgtgtgaaattgttatcc

SEQ ID NO: 6 - DNA, 40 bp, Artificial Sequence
  Oligonucleotide comprising 40nt stabilization strand.
  misc_feature 1: The “G” at position 1 is a modified base having Biotin attached thereto
ggttaacaatttcacacaggaggactggccgtcgttttac

SEQ ID NO: 7 - DNA, 21 bp, Artificial Sequence
  Primer M13R48
  misc_feature 1: The “G” at position 1 is a modified base having Biotin attached thereto
ggataacaatttcacacagga

SEQ ID NO: 8 - DNA, 17 bp, Artificial Sequence
  Primer M13-20
  misc_feature 1: The “G” at position 1 is a modified base having Biotin attached thereto
gtaaaacgacggccagt

SEQ ID NO: 9 - DNA, 87 bp, Artificial Sequence; identical to SEQ ID NO: 1
  Oligonucleotide comprising the 87nt specificity strand designated #22-40-25 of a synthetic antibody molecule.
  misc_feature 23, 24, 25, 26, 27, 28, 29, 30, 31, 32, 33, 34, 35, 36,37, 38, 39, 40, 41, 42, 43, 44, 45, 46, 47, 48, 49, 50, 51,52, 53, 54, 55, 56, 57, 58, 59, 60, 61, 62: n = any nucleotide or modified nucleotide

SEQ ID NO: 10 - DNA, 48 bp, Artificial Sequence; identical to SEQ ID NO: 2
  Oligonucleotide comprising the 48nt stabilization strand of a synthetic antibody molecule.

SEQ ID NO: 11 - DNA, 22 bp, Artificial Sequence
  Primer F22-5
  misc_feature 1: The “G” at position 1 is a modified base having FAM attached thereto
gtaaaacgacggccagtgtctc

SEQ ID NO: 12 - DNA, 25 bp, Artificial Sequence
  Primer Bio-21-4
  misc_feature 1: The “G” at position 1 is a modified base having Biotin attached thereto
ggataacaatttcacacaggaatct

SEQ ID NO: 13 - DNA, 78 bp, Artificial Sequence; identical to SEQ ID NO: 5
  Oligonucleotide comprising the 78nt specificity strand designated #17-40-21 of a synthetic antibody.
  misc_feature 18, 19, 20, 21, 22, 23, 24, 25, 26, 27, 28, 29, 30, 31,32, 33, 34, 35, 36, 37, 38, 39, 40, 41, 42, 43, 44, 45, 46,47, 48, 49, 50, 51, 52, 53, 54, 55, 56, 57: n = any nucleotide or modified nucleotide

SEQ ID NO: 14 - DNA, 48 bp, Artificial Sequence; identical to SEQ ID NO: 2
  Oligonucleotide comprising the 48nt stabilization strand of a synthetic antibody.

SEQ ID NO: 15 - DNA, 17 bp, Artificial Sequence
  Primer FM13-20
  misc_feature 1: The “G” at position 1 is a modified base having FITC attached thereto
gtaaaacgacggccagt

SEQ ID NO: 16 - DNA, 21 bp, Artificial Sequence
  Primer BioM13R48
  misc_feature 1: The “G” at position 1 is a modified base having Biotin attached thereto
ggataacaatttcacacagga

(19) United States
(12) Patent Application Publication
(10) Pub. No.: US 2007/0065809 A1
(43) Pub. Date: Mar. 22, 2007

(54) Compositions and methods screening using populations of surrogate antibodies

(75) Inventor: Stephen B. Friedman, Chapel Hill, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: SYNTHERICA CORPORATION, DURHAM, NC (US)

(21) Appl. No.: 10/545,495

(22) PCT Filed: Feb. 19, 2004
(86) PCT No.: PCT/US04/04903

§ 371 Date: Oct. 16, 2006

Related U.S. Application Data
(60) Provisional application No. 60/448,712, filed on Feb. 19, 2003.
(60) Provisional application No. 60/495,996, filed on Aug. 18, 2003.

(51) Int. Cl. C12Q 1/70; G01N 33/53

(52) U.S. Cl. 435/5; 435/7.92

(57) ABSTRACT

Methods and compositions for the detection, identification, and quantification of compounds of interest in a sample are provided. The compositions and methods include arrays and kits comprising a population of surrogate antibodies that bind compounds of interest. The surrogate antibodies can be immobilized on to a solid support by means of an interaction between a recognition nucleotide sequence comprised in the surrogate antibody and a capture nucleotide sequence comprised in a capture probe attached to the solid support. Also provided are methods of using the arrays for research and clinical diagnostics, drug discovery, environmental testing, food testing, and testing for the use of agents of biological and chemical warfare.

### FIELD OF THE MENTION

The present invention relates to the parallel detection, identification, and quantification of compounds of interest in a sample. More specifically, the present invention is directed to arrays of surrogate antibody molecules and methods for their use.

### BACKGROUND OF THE INVENTION

The detection, identification, and quantification of molecules in a complex mixture plays an essential role in a number of applications, including clinical diagnostics; pharmaceutical research and drug discovery; military applications, such as the detection and identification of agents used in biological and chemical warfare, law enforcement applications such as the detection of explosives and illicit narcotics, monitoring food and water safety, and testing for environmental pollutants and pathogens. In each of these applications, the identity and quantity of a specific analyte or group of analytes needs to be determined.

Current methods for detecting specific analytes in a complex mixture in a sample generally require the extraction of the sample into organic solvents, followed by analysis using gas or liquid chromatography or mass spectroscopy; however, these methods are slow and expensive. The development of compositions and methods that could be used to quickly and inexpensively detect, identify, and quantitate multiple different analytes in parallel would therefore provide a significant benefit.

In many applications it would also be beneficial to simultaneously detect different classes of analytes. For example, when monitoring an environmental sample for the presence of a particular pathogen or biological agent, it would be advantageous to simultaneously detect the presence of different classes of molecules that are associated with the presence of the pathogen or biological agent. Thus, there is a need in the art for methods for the parallel detection, identification, and quantitation of multiple classes of analytes in a sample.

Accordingly, there remains a need for methods and compositions for assaying in parallel complex mixtures of analytes, for identifying individual analytes in the mixture, and for identifying specific molecular recognition events involving one or more compounds of interest.

### SUMMARY OF THE INVENTION

The present invention provides methods and compositions for the detection, identification, and quantification of compounds in a sample. The methods and compositions are useful in a number of applications, including research and clinical diagnostics, drug discovery, environmental testing, food testing, and testing for the use of agents of biological and chemical warfare.

The methods of the invention include a method for detecting a ligand of interest in a population of test ligands. The method comprises contacting a population of test ligands with a population of surrogate antibody molecules under conditions that allow for the formation of a binding partner complex between at least one of the surrogate antibody molecules and at least one of the test ligands, to thereby form a binding complex between the test ligand and at least one surrogate antibody. The surrogate antibody molecules used in the method comprise a binding pocket that is formed by the interaction of a specificity strand and a stabilization strand. In some embodiments, the surrogate antibodies further comprise at least one oligonucleotide tail comprising a recognition nucleotide sequence, where the recognition nucleotide sequence is known and is unique to the particular surrogate antibody.

The binding partner complex comprising the ligand of interest and one or more specifically bound surrogate antibody molecules is contacted with an array comprising a population of capture probes. The capture probes are attached to a discrete known location of a solid support, and comprise a capture nucleotide sequence that is complementary to a recognition sequence comprised within an oligonucleotide tail of at least one surrogate antibody. The binding partner complex is contacted with the array under conditions that allow for the hybridization of the recognition sequence of an oligonucleotide tail of the surrogate antibody with the complementary capture nucleotide sequence of the corresponding capture probe on the solid support. In some embodiments, the binding partner complex is contacted with the array in the presence of the unbound surrogate antibody molecules and unbound test ligands. In other embodiments, the unbound surrogate antibody molecules and unbound test ligands are removed prior to contacting the binding partner complex with the array. The binding partner complex bound to the capture probe is then detected.

In an alternate embodiment, the method for detecting a ligand of interest in a population of test ligands comprises providing an array having 1) a population of capture probes attached to discrete known locations on a solid support, where the capture probes comprise a capture nucleotide sequence that is known and unique; and

2) a surrogate antibody molecule having at least one oligonucleotide tail comprising a recognition nucleotide sequence, where the recognition nucleotide sequence is known and unique to the particular surrogate antibody, and where the recognition nucleotide sequence is complementary to and forms a duplex with a capture nucleotide sequence. The surrogate antibody molecules used in the method comprise a binding pocket formed by the interaction of a specificity strand and a stabilization strand. The array is contacted with a population of test ligands under conditions that allow for the formation of a binding partner complex between at least one of the surrogate antibody molecules attached to the array and at least one ligand of interest. The binding partner complex is then detected to thereby detect the ligand of interest.

The specificity strand of the surrogate antibody molecules of the invention comprises a specificity domain flanked by a first constant region and a second constant region. The stabilization strand comprises a first stabilization domain that interacts with the first constant domain of the specificity strand and a second stabilization domain that interacts with the second constant domain of the specificity strand. In some embodiments, the specificity strand and the stabilization strand are found in distinct, non-contiguous strands. In other embodiments of the invention, the specificity domain, first and second constant region, and first and second stabilization domains are comprised within the same, contiguous strand. In some embodiments, the stabilization strand comprises an amino acid sequence. In other embodiments, the stabilization strand comprises a nucleotide sequence. In still other embodiments, the stabilization strand comprises a polymer of nucleotide-specific binding compounds.

The ligand of interest is detected by detecting the binding partner complex formed by the interaction between the ligand of interest and the surrogate antibody molecule. In some embodiments, the binding partner complex bound to the array is detected by a method selected from the group consisting of: a) detecting the signal from a fluorescent group attached to the surrogate antibody molecule; b) detecting the signal from a fluorescent group attached to the ligand of interest; c) detecting a change in a fluorescent signal, where the change in the fluorescent signal results from the physical proximity of a fluorescent group found on the surrogate antibody molecule and a fluorescence modifying group found on the ligand of interest; d) detecting a change in a signal emitted by a reporter group (e.g. fluorophore, chromophore) conjugated to the ligand of interest upon formation of a binding complex with the surrogate antibody; e) contacting the binding partner complex with a secondary molecule, where the secondary molecule contains a detectable label and binds specifically to the surrogate antibody molecule; f) contacting the binding partner complex with a secondary molecule, where the secondary molecule contains a detectable label and binds specifically to the ligand of interest; g) detecting the presence of a radioactive labeling group attached to the surrogate antibody molecule; h) detecting the presence of a radioactive labeling group attached to the ligand of interest; i) detecting the presence of an enzymatic labeling group attached to the surrogate antibody molecule; j) detecting the presence of an enzymatic labeling group attached to the ligand of interest; k) detecting a change in refractive index caused by the hybridization of the binding partner complex to the capture probe; 1) detecting a change in electrical conductance caused by the hybridization of the binding partner complex to the capture probe; m) detecting a change in potential caused by the hybridization of the binding partner complex to the capture probe; and n) detecting a change in resistivity caused by the hybridization of the binding partner complex to the capture probe

The present invention also provides a method of producing an array useful for detecting and identifying ligands of interest, and in diagnostics. In one embodiment, the method comprises providing a solid support, and attaching to the solid support a population of capture probes, where the capture probes are attached to discrete, known locations on the solid support, and the capture probes comprise a known and unique capture nucleotide sequence. The solid support is then contacted with a surrogate antibody having at least one oligonucleotide tail comprising a known recognition nucleotide sequence where the recognition sequence is unique to the particular surrogate antibody and where the recognition sequence is complementary to, and capable of hybridizing with at least one capture nucleotide sequence. The solid substrate comprising the capture probes is contacted with the surrogate antibodies under conditions that allow for the hybridization of the capture nucleotide sequence and the recognition nucleotide sequence.

Compositions of the present invention include an array and kits comprising the array and instructions for use in a method of detecting or identifying a test ligand. In one embodiment the array comprises 1) a solid support having attached thereto a population of capture probes, where the capture probes comprise known, unique capture nucleotide sequences; and 2) a surrogate antibody having an oligonucleotide tail having a known recognition sequence, where the recognition sequence is unique to the particular surrogate antibody specificity and is complementary to and forms a duplex with at least one capture nucleotide sequence on the solid support.

Additional compositions include a population of surrogate antibody molecules. The surrogate antibody molecules comprising a specificity region and further comprise an oligonucleotide tail comprising a recognition nucleotide sequence, where the recognition nucleotide sequence is known and unique to the particular surrogate antibody specificity.

Further compositions comprise a kit comprising 1) a population of surrogate antibody molecules wherein the population of surrogate antibody molecules is characterized as having a unique, known oligonucleotide tail on each surrogate antibody of the population; and, 2) a substrate, wherein affixed to the substrate is a population of nucleotide sequences wherein each of the nucleotide sequences in the population is unique; comprises a complementary oligonucleotide tail; is attached to a discrete known location of the substrate; and, wherein upon contacting said population of surrogate antibody molecules with the substrate, the hybridization of the oligonucleotide tail of the surrogate antibody with the complementary oligonucleotide tail of the support occurs.

### BRIEF DESCRIPTION OF THE FIGURES

FIG. 1 is a diagram representing a surrogate antibody (SAb) molecule that contains one or more stabilization regions (ST) composed of juxtaposed oligonucleotide strands (A, A′, D, and D′) that border one or more specificity regions (SP) composed of a sequence of nucleotides that form a ligand-binding cavity. In this embodiment, the upper stand (specificity strand) comprises a specificity region (SP) flanked by two constant regions (A and D). The lower strand (stabilization strand) comprises a spacer region flanked by two stabilization regions (A′ and D′) that interact with the respective constant region (A and D).

FIGS. 2A and 2B are diagrams representing two embodiments of surrogate antibody molecules that include multiple specificity regions (SP region loops), stabilization regions (ST), and spacer regions (S).

FIGS. 3A-3D are diagrams representing four embodiments of surrogate antibody molecules that contain multiple specificity regions (SP region loops), stabilization regions (ST), and spacer regions (S) and that collectively provide multi-dimensional ligand binding.

FIG. 4 is a schematic illustration showing the binding of target ligands to surrogate antibody molecules containing SP region loops of varying sizes.

FIG. 5 is a schematic illustration showing surrogate antibody capacity to enhance binding affinity and specificity relative to natural antibodies.

FIG. 6 is a schematic illustration of one method of preparing surrogate antibodies.

FIG. 7 provides a non-limiting method for amplifying a surrogate antibody. In this embodiment, “F48” comprises the stabilization strand (SEQ ID NO: 1) and “F22-40-25 (87)” comprises the specificity strand (SEQ ID NO: 2). The stabilization strand comprises a 5 nucleotide mis-match (shaded box) to the specificity strand. This mis-match in combination with the appropriate primers (B21-40, SEQ ID NO:3; and F17-50, SEQ ID NO:4) will prevent amplification of the stabilization strand during PCR amplification. More details regarding this method are found in Example 4.

FIG. 8 illustrates the electrophoretic mobility of the surrogate antibody that were assembled using different combinations of specificity and stability primers.

FIG. 9 characterizes the surrogate antibodies using a denaturing gel to verify the duplex nature of the molecule.

FIG. 10 illustrates the selection and enrichment of the surrogate antibodies to the BSA-PCT (BZ101 congener) conjugate through 8, 9 and 10 cycles. Signal/Negative control represents as a percent, the amount of surrogate antibody bound to the target verses the amount of surrogate antibody recovered when the target is absent (negative control).

FIG. 11 illustrates the unique congener response profiles the array would produce for selected Aroclors®.

FIG. 12 illustrates the selection and enrichment of the surrogate antibodies to IgG. Signal/Negative control represents as a percent, the amount of surrogate antibody bound to the target verses the amount of surrogate antibody recovered when the target is absent (negative control).

FIG. 13 illustrates an embodiment of the invention in which a ligand of interest is contacted with two surrogate antibodies that bind two separate epitopes on the ligand of interest. Each of the surrogate antibodies contains the same recognition sequence, allowing the binding partner complex formed between the ligand of interest and the surrogate antibodies to be immobilized on an array of the invention by means of an interaction between the recognition sequence comprised in the surrogate antibodies and the capture nucleotide sequences comprised within the capture probes, which are attached to discrete, know regions of the array.

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The present invention provides compositions and methods for detecting, identifying, and/or quantifying analytes in a sample. The compositions of the invention rely on the use of surrogate antibodies that are capable of binding to a wide variety of analytes or ligands. The sample is contacted with a population of surrogate antibodies under conditions that allow the surrogate antibodies to bind to one or more ligands in the sample to form a binding partner complex. In order to detect, identify, and/or quantitate the level of the ligand in the sample, the binding partner complex is immobilized onto an array by means of an interaction between a “recognition” nucleotide sequence in the surrogate antibody and a “capture” nucleotide sequence attached at a discrete, known location in the array. In addition to their use in the detection of diverse types of ligands in a sample, the arrays may also be used to generate “ligand profiles” that are characteristic of a particular type of sample and may be used to identify a particular sample. The arrays of the invention are also useful in screening assays.

The samples or “populations of test ligands” used in the methods of the invention may be any sample or population of interest. For example, the population of test ligands may be derived from an environmental sample, a food sample, a pharmaceutical sample, a water sample, or an industrial sample. Alternatively, the population of test ligands may be derived from a biological sample such as a virus, cell, tissue, organ, or organism including, but not limited to, a cellular extract, tissue or organ lysates or homogenates, or body fluid samples, such as blood, urine, cerebrospinal fluid saliva, sputum, feces, amniotic fluid, or wound exudate. The population of test ligands may comprise any number of types of test ligands. For example, in some embodiments of the invention, the population of test ligands contains a single type of test ligand, while in other embodiments, the population of test ligands is a complex mixture containing a number of types of test ligands.

The surrogate antibodies utilized in the compositions and methods of the present invention are capable of binding a wide variety of ligands. Accordingly, ligands of interest of the invention may be any ligands that interact with a surrogate molecule of the invention. Examples of ligands of interest include, but are not limited to, organic molecules, inorganic molecules, immunological haptens, environmental pollutants and toxins (e.g., polychlorinated biphenyls, dioxins, polyaromatic hydrocarbons), contaminants in gasoline, agents used in biological or chemical warfare, natural or surrogate polymers, carbohydrates, polysaccharides, muccopolysaccharides, glycoproteins, enzymes, antigens, molecules (e.g. proteins, nucleic acid molecules, carbohydrates, or metabolites) derived from any source, such as a cell, a eukaryotic cell, a bacteria, or a virus, therapeutic agents, illicit drugs and substances of abuse (e.g., narcotics) hormones, peptides, polypeptides, prions, and nucleic acids. A ligand can also be a cell or its constituents, for example, a pathogen one or more cellular organelles. The ligand can also be any cell type of interest, at any developmental stage, and having various phenotypes. For example, the surrogate antibody can be developed to bind a variety of tumor cells, including, but not limited to, colon tumor cells, breast cancer cells, prostate tumor cells, etc. Where the ligand of interest is a pathogen, surrogate antibodies that specifically recognize a particular strain of the pathogen may be used. Additional ligands of interest include molecules whose levels are altered in tumors (i.e., growth factor receptors, cell cycle regulators, angiogenic factors, and signaling factors). Accordingly, the surrogate antibody molecules of the invention can be produced for the detection of any ligand of interest.

Accordingly, the compositions and methods find use in a number of applications that require the presence of a specific analyte in a sample, including environmental testing, food testing, and testing for the use of explosives or agents of biological and chemical warfare research. The methods and compositions of the invention are also useful in clinical diagnostics; pharmaceutical research and drug discovery,

### Compositions

I. Surrogate Antibody Molecules

The methods of the invention employ populations of surrogate antibody molecules. A detailed description of such surrogate antibody molecules can be found, for example, in U.S. Provisional Application No. 60/358,459 filed Feb. 19, 2002, and the U.S. utility application entitled “Surrogate Antibodies and Methods of Preparation and Use Thereof” filed concurrently with the present application, both of which are herein incorporated by reference in their entirety. In some embodiments, the surrogate antibody molecules in the population of the present invention comprise at least one oligonucleotide tail having a known recognition sequence that is unique to a particular surrogate antibody specificity. A more detailed description of the structure of the surrogate antibody molecule and the populations of surrogate antibody molecules for use in the methods of the invention are provided below.

As used herein, a surrogate antibody refers to a class of molecules that contain discrete nucleic acid structures or motifs that enable selective binding to target molecules. In one embodiment, the surrogate antibody comprises a specificity strand and a stabilization strand. The specificity strand comprises a nucleic acid sequence having a specificity region flanked by a first constant region and a second constant region. The stabilization strand comprises a first stabilization region that interacts with the first constant region and a second stabilization region that interacts with the second constant region. The interaction of the stabilization strand and the specificity strand results in the formation of a molecule that is capable of interacting with a desired ligand. The sequence of the specificity domain (both the primary and secondary structure in the final surrogate antibody molecule) will influence the ligand binding specificity of the antibody.

The specificity domains and stabilization domains of the surrogate antibodies allow for the formation of surrogate antibodies having a large number of sequences and shapes. The vast diversity of possible binding pockets created allows a desired function and binding affinity to be created. That is, the surrogate antibodies provide sufficient physical and chemical diversity to provide tight and specific binding to most targets.

The invention encompasses isolated or substantially isolated surrogate antibody compositions. An “isolated” surrogate antibody molecule is substantially free of other cellular material, or culture medium, chemical precursors, or other chemicals when chemically synthesized. A surrogate antibody that is substantially free of cellular material includes preparations of surrogate antibody having less than about 30%, 20%, 10%, 5%, (by dry weight) of contaminating protein or nucleic acid. In addition, if the surrogate antibody molecule comprises nucleic acid sequences homologous to sequences in nature, the “isolated” surrogate antibody molecule is free of sequences that may naturally flank the nucleic acid (i.e., sequences located at the 5′ and 3′ ends of the nucleic acid) in the genomic DNA of the organism from which the surrogate antibody has homology.

As used herein, nucleic acid means DNA, RNA, TNA, single-stranded or double-stranded and any chemical modifications thereof. A surrogate antibody can be composed of double-stranded RNA, single-stranded RNA, single stranded DNA, double stranded DNA, a hybrid RNA-DNA double strand combination, a hybrid TNA-DNA, a hybrid TNA-RNA, a hybrid amino acid/RNA, amino acid/DNA, amino acid/TNA or any combination thereof provided that the interacting regions that allow for the stabilization of one or more loop structures. It is further recognized that the nucleic acid sequences include naturally occurring nucleotides and surrogateally modified nucleotides.

A. The Specificity Strand

As used herein, the specificity strand of the surrogate antibody comprises a nucleic acid molecule having a specificity region flanked by two constant regions. By the phrase “flanked by” it is intended that the constant regions may either be immediately adjacent to the specificity region or may be found 5′ and 3′ to the specificity region but are separated by a spacer sequence. The specificity region functions as a ligand binding domain, while the constant domains interact with the stabilization domains found on the stabilization strand to thereby allow the specificity domain to form a ligand binding cavity.

The specificity strand comprises a nucleic acid sequence composed of ribonucleotides, modified ribonucleotides, deoxyribonucleotides, modified deoxyribonucleotides, (3′,2′-α-L-threose nucleic acid (TNA), modified TNA or any combination thereof. See, for example, Chaput et al. (2003) J. Am. Chem. Soc. 125:856-857, herein incorporated by reference. Possible modifications include the attachment of a functional moiety or molecule to the nucleotide sequence. The modification can be at the 5′ end, the 3′ end, or both the 5′ end and the 3′ end of the sequence. The functional moiety may also be added to individual nucleotides or amino acid residues anywhere in the strand, attached to all or a portion of the pyrimidines or purines present in the strand, or attached to all or a portions of a given type of nucleotide. While various modifications to DNA and RNA residues are known in the art, examples of some modifications of interest to the surrogate antibodies of the present invention are discussed in further detail below.

The specificity strand and its respective domains (i.e., the constant domains and the specificity domains and, in some embodiments, the spacer regions) can be of any length, so long as the strand can form a surrogate antibody as described elsewhere herein. For example, the specificity strand can be between about 10, 50, 100, 200, 400, 500, 800, 1000, 2000, 4000, 8000 nucleotides or greater in length. Alternatively, the specificity strand can be from about 15-80, 80-150, 150-600, 600-1200, 1200-1800, 1800-3000, 3000-5000 or greater. The constant domains and the specificity domains can be between about 2 nucleotides to about 100 nucleotides in length, between about 20 to about 50 nucleotides in length, about 10 to about 90 nucleotides in length, about 10 to about 80 nucleotides in length, about 10 to about 60 nucleotides in length, or about 10 to about 40 nucleotides in length.

While a surrogate antibody molecule does not require a spacer region in the specificity region, if a spacer region is present, it can be of any length. For example, a spacer region can be about 2 nucleotides to about 100 nucleotides in length, between about 20 to about 50 nucleotides in length, about 10 to about 90 nucleotides in length, about 10 to about 60 nucleotides in length, or about 10 to about 40 nucleotides in length. In yet other embodiments, the spacer region could comprise groups other than one or more nucleotides. Any group could be used so long as it provides the desired spacing to form the surrogate antibody molecule. For example, a spacer region could comprise a phosphate moiety.

In some embodiments, the specificity strand or its components (the constant regions or the specificity region) have significant similarity to naturally occurring nucleic acid sequences. In other embodiments, the nucleic acid sequence can share little or no sequence identity to sequences in nature. In still other embodiments, the nucleic acid residues may be modified as described elsewhere herein.

B. The Stabilization Strand

The surrogate antibody further comprises a stabilization strand. The stabilization strand comprises stabilization domains that are capable of interacting with the constant domains of the specificity strand and thereby stabilize the ligand-binding cavity of the specificity domain. Accordingly, the stabilization strand can comprise, for example, an amino acid sequence, a nucleic acid sequence, or any of various polymers including any cationic polymer, cyclodextrin polymer, or a polymer having an appropriately charged intercalating agent such as lithium bromide or ethidium bromide.

It is recognized that the stabilization domains in a surrogate antibody can be identical (i.e., the same nucleotide sequence or peptide sequence) or non-identical, so long as each stabilization region interacts with their corresponding constant region in the specificity strand. In addition, the interaction between the constant regions and the stabilization regions may be direct or indirect. The interaction will further be such as to allow the interaction to occur under a variety of conditions including under the desired ligand-binding conditions.

In some embodiments, components of the surrogate antibodies (i.e., the stabilization strand and its respective domains) are not naturally occurring in nature. In others embodiments, they can have significant similarity to a naturally occurring nucleic acid sequences or amino acid sequences or may actually be naturally occurring sequences. One of skill in the art will recognize that the length of the stabilization domain will vary depending on the type of interaction required with the constant domains of the specificity strand. Such interactions are discussed in further detail elsewhere herein.

A stabilization domain may comprise any amino acid sequence that is capable of interacting with the nucleic acid sequence of the constant domains of the specificity strand. For example, an amino acid sequences having DNA binding activity (i.e., zinc finger binding domains (Balgth et al. (2001) Proc. Natl. Acad. Sci. 98:7158-7163; Friesen et al. (1998) Nature Structural Biology, Tang et al (2001) J. Biol. Clien. 276:19631-9; Dreier et al. (2001) J. Biol. Chem. 29466-79; Sera et al. (2002) Biochemistry 41:7074-81, all of which are herein incorporated by reference), helix-turn domains, and leucine zipper motifs (Mitra et al. (2001) Biochemistry 40:1693-9)) or polypeptides having lectin activity (e.g. monosaccharide binding activity or oligosaccharide activity) may be used for one or more of the stabilization domains. Accordingly, various polypeptides could be used, including transcription factors, restriction enzymes, telomerases, RNA or DNA polymerases, inducers/repressors or fragments and variants thereof that retain nucleic acid binding activity. See for example, Gadgil et al. (2001) J. Biochem. Biophys. Methods 49: 607-24. In other embodiments, the stabilization strand could include sequence-specific DNA binding small molecules such as polyamides (Dervan et al. (1999) Current Opinion Chem. Biol. 6:688-93 and Winters et al. (2000) Curr Opin Mol Ther 6:670-81); antibiotics such as aminoglycosides (Yoshhizawa et al (2002) Biochemistry 41:6263-70) quinoxaline antibiotics (Bailly et al. (1998) Biochem Inorg Chem 37:6874-6883; AT-specific binding molecules (Wagnarocoski et al (2002) Biochem Biophys Acta 1587:300-8); rhodium complexes (Terbrueggen et al. (1998) Inorg. Chem. 330:81-7). One of skill in the art will recognize that if, for example, a zinc finger binding domain is used in the stabilization strand, the corresponding nucleic acid binding site will be present in the desired constant region of the specificity strand. Likewise, if a polypeptide having lectin activity is used in the stabilization strand, the corresponding constant domain of the specificity strand will have the necessary modifications to allow for the desired interaction. When the stabilization domain comprises an amino acid sequence, any of the amino acid residues can be modified to contain functional moieties. Such modifications are discussed in further detail elsewhere herein.

In some embodiments the stabilization domain comprises a nucleic acid molecule, and the constant domains of the specificity strand are complementary to the stabilization domains. In this embodiment, the surrogate antibodies are formed when the stabilization strand and the specificity strand are hybridized together to allow for the appropriate interaction between the stabilization domains and the constant domains.

In one embodiment, the stabilization strand is longer than the specificity strand.

The stabilization strand can comprise any type of nucleotide, including for example, ribonucleotides, modified ribonucleotides, deoxyribonucleotides, modified deoxyribonucleotides or any combination thereof.

C. The Oligonucleotide Tail

In some embodiments of the methods and compositions of the present invention the surrogate antibodies comprise at least one oligonucleotide tail. The oligonucleotide tail comprises a recognition nucleotide sequence that is complementary to a capture nucleotide sequence of capture probe. The capture probes are attached to a solid substrate. The oligonucleotide tail can be made of any nucleotide base, including for example, ribonucleotides, modified ribonucleotides, deoxyribonucleotides, modified deoxyribonucleotides, TNA, modified TNA, or any combination thereof. The recognition nucleotide sequence will be of sufficient length and nucleotide composition to hybridize to the capture nucleotide sequence found in the corresponding capture probe. Accordingly, the recognition nucleotide sequence can be of any length, including from about 4 to about 500 nucleotides. In some embodiments, the recognition nucleotide sequence is from about 4 to about 100 nucleotides.

The oligonucleotide tails may be attached to any region of the surrogate antibody molecule. For example, a tail can be found attached to the specificity strand (i.e., either at the 5′ or 3′ end), the stabilization strand, or both the specificity strand and the stabilization strand. The method and location of attachment to the stabilization strand will vary depending on the composition of the strand. For instance, if the stabilization strand comprises an amino acid sequence, the tail can be attached to the amino or carboxy terminus or to any amino acid in between. If the stabilization domain is a nucleic acid, the tail could be attached to the 5′ or 3′ end.

In some embodiments of the invention, the surrogate antibodies comprise an oligonucleotide tail comprising a known and unique recognition sequence. By “unique” is intended that each surrogate antibody in the population that recognizes a different ligand in the population of test ligands has a novel or non-duplicated recognition nucleotide sequence. Thus, the recognition sequence is unique to the ligand specificity of the surrogate antibody molecule. By “known” is intended that the sequence of the recognition nucleotide sequence comprised in an oligonucleotide tail of a surrogate antibody molecule is known, allowing for identification of the specific surrogate antibody molecules and binding partner complexes. For example, in some embodiments of the invention, the surrogate antibody molecule is immobilized to array by means of an interaction with a capture probe. The capture probe is attached to a discrete, known location on the array and comprises a capture nucleotide sequence that is complementary to and hybridizes with the recognition nucleotide sequence found in an oligonucleotide tail of the surrogate antibody. Accordingly, by measuring the signal at a particular address on the array, it is possible to detect, identify, and quantitate a binding partner complex containing one or more surrogate antibody molecules having a particular ligand specificity. Furthermore, where the ligand specificity of the surrogate antibody is known, the ligand may be detected, identified, and quantitated by detecting the binding partner complex.

D. Forming a Surrogate Antibody Molecule

The surrogate antibody molecule of the present invention is formed by providing a specificity strand and a stabilization strand and contacting the specificity strand with the stabilization strand under conditions that allow for the first stabilization domain to interact with the first constant domain and the second stabilization domain to interact with the second constant domain. The specificity strand and stabilization strand are contacted under conditions that allows for the stable interaction of the stabilization domains and the constant domains. A population of surrogate antibodies can be formed using these methods.

As discussed below, conditions for forming the surrogate antibody molecule will vary depending on the ligand of interest and the intended applications. One of skill will be able to empirically determine the appropriate conditions for the desired application. For example, if the intended application is to occur under physiological conditions the formation of the antibody may be performed at pH 7.4 at a physiological salt concentration (i.e., 280-300 milliosmols) and a temperature of about 37° C.

When the stabilization domains comprise a nucleic acid sequence, the nucleotide sequences of the constant domains and the stabilization domains will be such as to allow for hybridization under the desired conditions (e.g., under ligand-binding conditions). Furthermore, the stabilization domains and constant domains are designed to allow for assembly such that the first constant domain preferentially hybridizes to the first stabilization domain and the second stabilization domain referentially hybridizes to the second constant domain. Accordingly, the interaction of the specificity strand and stabilization strand promotes sequence-directed self-assembly of the surrogate antibody.

In one embodiment, the surrogate antibody molecule is designed to result in a Tm for of each stabilization/constant domain interaction to be approximately about 15 to about 25° C. above the temperatures of the intended application (i.e., the desired ligand binding conditions). Accordingly, if the intended application is a therapeutic application or any application performed under physiological conditions, the Tm can be about 37° C.+about 15° C. to about 37° C.+25° C. (i.e., 49° C., 50° C., 52° C., 54° C., 55° C., 56° C., 58° C., 60° C., 62° C., 64° C., or greater). If the intended application is a diagnostic assay conducted at room temperature, the Tm can be (25° C.+about 15° C.) to about (25° C.+about 25° C.) (i.e., 38° C., 40° C., 41° C., 42° C., 43° C., 44° C., 46° C., 48° C., 50° C., 52° C., 53° C. or greater). Equations to measure Tm are known in the art. A preferred program for calculating Tm comprises the OligoAnalyzer 3.0 from IDT BioTools© 2000. It is recognized that any temperature can be used the methods of the invention. Thus, the temperature of the ligand binding conditions can be about 5° C., 10° C., 15° C., 16° C., 18° C., 20° C., 22° C., 24° C., 26° C., 28° C., 30° C., 32° C., 34° C., 38° C., 40° C., 42° C., 44° C., 46° C., 48° C., 50° C., 52° C., 54° C., 56° C., 58° C., 60° C. or greater.

Alternatively, the stabilization domains and the respective constant domains are designed to allow about 40% to about 99%, about 40% to about 50%, or about 50% to about 60%, about 60% to about 70%, about 70% to about 80%, about 85%, about 90%, about 95%, about 98% or more of the surrogate antibody population to remain annealed under the intended ligand binding conditions. Various methods, including gel electrophoresis, can be used to determine the % formation of the surrogate antibody. See Experimental section. In addition, calculation for this type of determination can be found, for example, in Markey et al. (1987) Biopolymers 26:1601-1620 and Petersteim et al. (1983) Biochemistry 22:256-263, both of which are herein incorporated by reference.

The relative concentration of the specificity strand and the stabilization strand can vary so long as the ratio will favor the formation of the surrogate antibody. Such conditions include providing an excess of the stabilization strand.

The constant domains and stabilization domains can have any desired guanine/cytosine content, including, for example, about 0%, 5%, 10%, 15%, 20%, 25%, 30%, 35%, 40%, 45%, 50%, 55%, 60%, 65%, 70%, 75%, 80%, 85%, 90%, 95%, or 100% guanine/cytonsine.

The stabilization domains and, where applicable, spacer regions, of the stabilization strand can be of any length, so long as the stabilization strand can form a surrogate antibody as described herein. For example, the stabilization strand can be between about can be between about 8, 10, 50, 100, 200, 400, 500, 800, 1000, 2000, 4000, 8000 nucleotides or greater in length. Alternatively, the stabilization strand can be from about 15-80, 80-150, 150-600, 600-1200, 1200-1800, 1800-3000, 3000-5000 or greater.

The stabilization domains can be between about 2 nucleotides to about 100 nucleotides in length, between about 20 to about 50 nucleotides in length, about 10 to about 90 nucleotides in length, about 10 to about 60 nucleotides in length, or about 10 to about 40 nucleotides in length. If a spacer region is present in the stabilization strand, this region can be about 1 nucleotides to about 100 nucleotides in length, between about 5 to about 50 nucleotides in length, about 10 to about 90 nucleotides in length, about 10 to about 60 nucleotides in length, or about 10 to about 40 nucleotides in length. Alternatively, as discussed elsewhere herein, the spacer can comprise one or more molecule including, for example, a phosphate moiety. The length and guanine/cytosine content of each domain can vary so long as the interaction between the constant domains and the stabilization domain is sufficient to stabilize the antibody structure and produce a stable binding loop (specificity region). In addition, the stabilization strand can be linear, circular or globular and can further contain stabilization domains that allow for multiple (2, 3, 4, 5, 6, or more) specificity strands to interact.

The known oligonucleotide structures or motifs that are involved in non-Watson-Crick type interactions, such as hairpin loops, symmetric and asymmetric bulges, pseudo-knots and combinations thereof, have been suggested in the art to form from nucleic acid sequences of no more than 30 nucleotides. However, it has now been found that larger loop structures can be stabilized in the surrogate antibodies described herein. The specificity region can include between about 10 and 90 nucleotides, between about 10 and 80, between 10 and 60, or between 10 and 40 nucleotides. These stabilized binding cavities provide sites for hydrophobic binding and contribute to increased binding affinity in a manner that mimics the major force implicated in natural antibody binding. As such the ligand-binding cavity of the surrogate antibody can include one or more hairpin loops, asymmetric bulged hairpin loops, symmetric hairpin loops, and pseudoknots.

One of skill in the art will recognize that each stabilization domain and corresponding constant domain will preferably be designed to maximize the stability of the interactions under the desired conditions and thereby maintain the structure of the surrogate antibody. See, for example, Guo et al. (2002) Nature Structural Biology 9:855-861 and Nair et al. (2000) Nucleic Acid Research 28:1935-1940. Methods to measure the stability or structure of the surrogate antibody molecules are known. For example, surface plasmon resonance (BIACORE) can be used to determine kinetic values for the formation of surrogate antibody molecules (BIACORE AB). See, for example, U.S. Pat. Nos. 5,955,729, 6,207,381, and 6,289,286, each of which is incorporated in its entirety by reference. Other techniques of use include NMR spectroscopy and electrophoretic mobility shift assays. See, Nair et al. (2000) Nucleic Acid Research 9:1935-1940, herein incorporated by reference. It is recognized, however, that the stabilization domain and constant domain need not have 100% sequence identity with one another. All that is required is that they bind in a directed fashion to form a stable structure when exposed to ligand-binding conditions. Generally, this requires that the stabilization domain and the corresponding complement of the constant domain have at least 80%, at least 85%, at least 90%, at least 95%, at least 96%, at least 97%, or at least 98% sequence identity. In addition, the interaction between the stabilization domain and the constant domain may require at least 5 consecutive complementary nucleotide residues in the stabilization domain and the corresponding constant domain.

As used herein, “sequence identity” or “identity” in the context of two nucleic acid refers to the nucleotides in the two sequences that are the same when aligned for maximum correspondence over a specified comparison window. “Percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

Methods for sequence alignment and for determining identity between sequences are well known in the art. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Polypeptide Sequence and Structure 5:Suppl. 3 (National Biomedical Research Foundation, Washington, D.C.). With respect to optimal alignment of two nucleotide sequences, the contiguous segment of the constant or stabilization domain may have additional nucleotides or deleted nucleotides with respect to the corresponding constant/stabilization nucleotide sequence. The contiguous segment used for comparison to the reference nucleotide sequence will comprise at least 5, 10, 15, 20, or 25 contiguous nucleotides and may be 30, 40, 50, 100, or more nucleotides. The percent identity between the two sequences is a function of the number of identical positions shared by the sequences, taking into account the number of gaps, and the length of each gap, which need to be introduced for optimal alignment of the two sequences.

The determination of percent identity between two sequences can be accomplished using a mathematical algorithm. Percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic.

When the specificity strand and the stabilization strand of the surrogate antibody comprise nucleic acid sequences, the surrogate antibodies can be formed by placing the first and second strand in solution, heating the solution, and cooling the solution under conditions such that, upon cooling, the first and second strand anneal and form the antibody. Any hybridization that could occur between two first strands or two second strands would not be stable because of the significantly weaker affinity coefficients relative to the designed multi-nucleotide complementation bonds designed into each of the specificity regions and the corresponding constant domains.

E. Diverse Structures of Surrogate Antibodies

Surrogate antibodies are a class of molecules having a nucleic acid sequence arranged to form a stable binding cavity that provides specific ligand binding through conformational complementarity to the ligand, and affinity through cooperative hydrophobic, electrostatic, Van der Waals-forces, and/or hydrogen binding, except where the target/ligand is a nucleic acid composition and binding by means of Watson/Crick base pairing or triple helical association is desired. See, for example, Riordan et al. (1991) Nature 350:442-443. Accordingly, a diverse number of surrogate antibodies structures can be formed. In one embodiment, the surrogate antibodies described herein can include one or more distinct specificity strands having one or more than one specificity domains, wherein each specificity domain is flanked by constant domains. Surrogate antibodies of the invention can therefore have 1, 2, 3, 4, 5 or more specificity domains. Thus the surrogate antibody molecules can be formed using multiple oligonucleotides. See, for example, FIGS. 2 and 3. Accordingly, the surrogate antibody can be “multi-valent” and thereby contain multiple specificity domains contained on one specificity strand or on multiple distinct strands. Thus, the specificity domains of a multi-valent surrogate antibody can be the same nucleotide sequence and of the same size and recognize the same ligand epitope. In other embodiments, the specificity domains can be different and thus form “pluri-specific” surrogate antibodies. The pluri-specific antibody will bind different ligands or different regions/epitopes of the same ligand. Accordingly, each specificity domain can be designed to bind the same target/ligand or to different targets/ligands. In this way, a surrogate antibody can simultaneously bind two common determinates on a single cell, or be able to bind a compound in two distinct orientations. For example, an antibody can bind a particular receptor in a preferred binding site and also in an allosteric position. Alternatively, the surrogate antibody can bind a particular pair of receptors on a given cell surface thereby increasing affinity through cooperative binding interactions or form a bridge between molecules or cells.

In another embodiment, the surrogate antibody molecule can comprises a spacer region on either the stabilization strand or the specificity strand that eliminates stress in the molecule and/or stearically optimizes binding to adjacent targets and/or modifies the size and/or conformation of the specificity domain. Thus, the spacer region can be used to eliminate bond stress in molecules and provide diversity to the size and shape of the binding cavity. Accordingly, the surrogate antibody molecule can comprises one or more spacer regions having a common number of residues and sequence or a different number of residue and sequence.

It is further recognized that when the stabilization strand and the specificity strand comprise a nucleic acid sequence, the strands can be contained on the same contiguous (covalently linked) strand of nucleic acid, or on distinct, non-contiguous (non covalently-linked) nucleic acid strands. Thus, in some embodiments, the surrogate antibodies are formed from a single nucleic acid strand comprising a) a first constant domain, a specificity domain, a second constant domain, a first spacer region, a second stabilization domain that is capable of hybridizing to the second constant domain, a second spacer region, and a first stabilization domain that is capable of hybridizing to the first constant domain. In one embodiment, each domain contains between about one to about twenty nucleotides. The nucleic acid strands can be linear or cyclic, so long as the specificity region forms a loop structure when the stabilization domains and the constant domains are hybridized.

Alternatively, the specificity strands and stabilization strands need not be linked by a covalent interaction. In some embodiments the specificity strands and stabilization strands can be contained on non-contiguous or distinct (non-covalently linked) nucleic acid strands and interact (directly or indirectly) via non-covalent interactions. In this embodiment, both the specificity strand and the stabilization strand will have a 3′ and 5′ termini. Accordingly, the invention relates to a ligand-binding surrogate antibody molecule comprising an assembly of two or more single stranded RNA oligonucleotide strands, two or more single stranded DNA oligonucleotide strands, INA, two or more TNA oligonucleotide strands, or a combination of two or more single stranded RNA, DNA, and/or TNA strands.

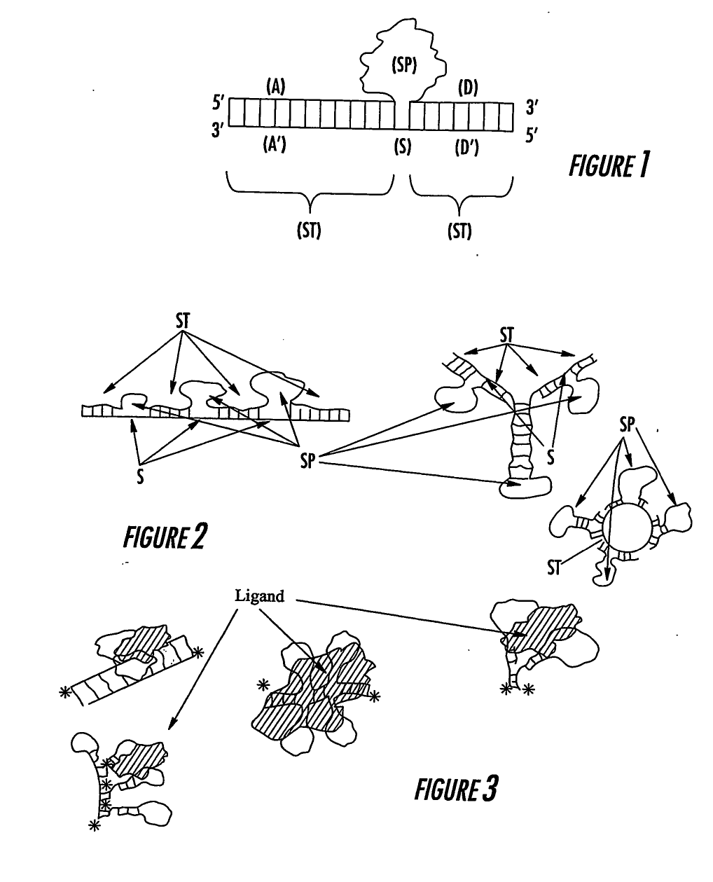

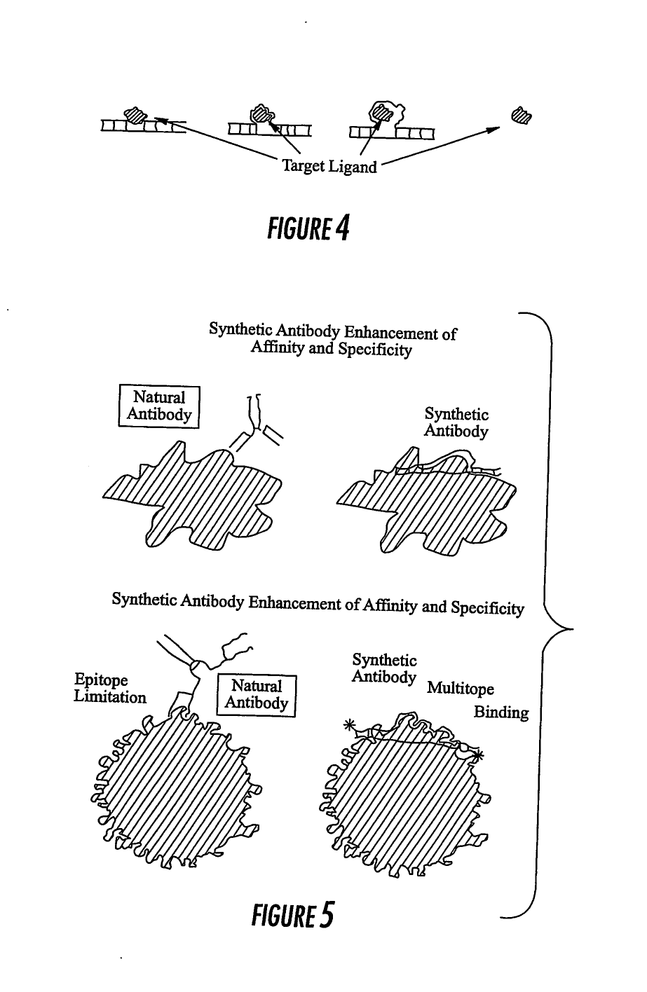

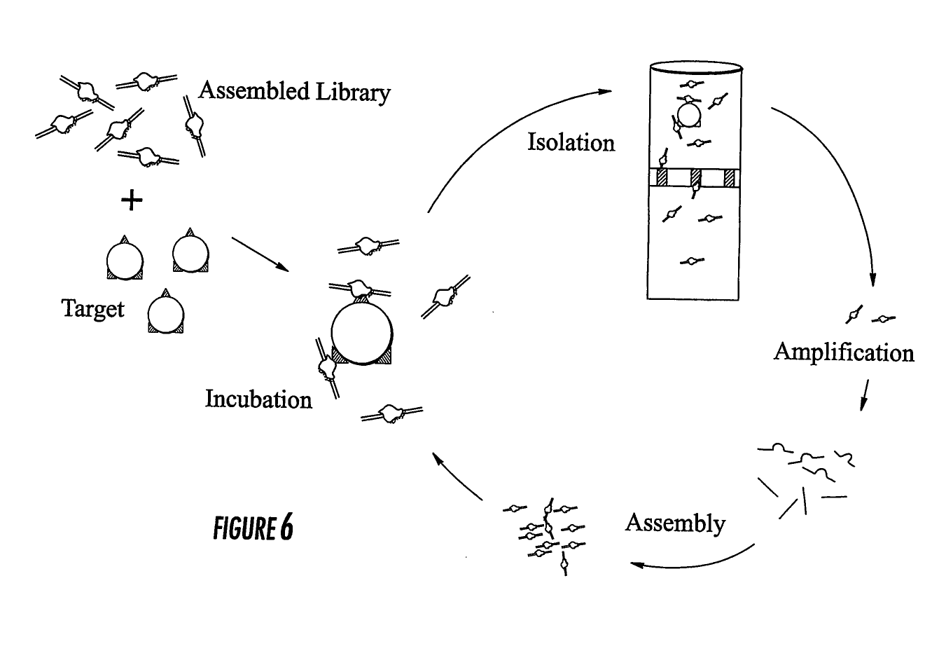

Representations of various types of surrogate antibody molecules are shown in FIG. 1. FIG. 2 shows two embodiments of surrogate antibody molecules that include multiple specificity regions. In one embodiment, the surrogate antibody molecules include multiple specificity domains (SP), stabilization domains (ST) and spacer regions (S) that collectively provide multi-dimensional ligand binding. These types of molecules are shown, for example, in FIGS. 3a-3d.

The stabilization strand and specificity strand may contain naturally-occurring nucleotides and amino acid residues or surrogateally-modified nucleotides and residues. Modifications encompassed by the present invention include the attachment of one or more functional moieties. As discussed in further detail below, the functional moiety can be attached to the stabilization or specificity strand via covalent or non-covalent interactions. Possible modifications of nucleotides include, but are not limited to, the attachment of amines, diols, thiols, phophorothioate, glycols, fluorine, hydroxl, fluorescent compounds (e.g. FITC), avidin, biotin, aromatic compounds, alkanes, and halogens. Further modifications of interest include, but are not limited to, modifications at cytosine exocyclic amines, substitution of 5-bromo-uracil (Golden et al. (2000) J. of Biotechnology 81:167-178), backbone modifications, methylations, unusual base-pairing combinations and the like. See, for a review, Jayasena et al. (1999) Clinical Chemistry 45:1628-1650.

Those of skill in the art are aware of numerous modifications to nucleotides and to phosphate linkages between adjacent nucleotides that render them resistant to cleavage by nucleases (Uhlmann et al. (1990) Chem Rev. 90:543-98 and Agraul et al. (1996) Trends Biotechnology 14:147-9 and Usman et al. (2000) The Journal of Clinical Investigations 106:1197-1202). Such functional moieties include, for example, modifications at the 2′ position of the sugars (Hobbs et al. (1973) Biochemistry 12:5138-45 and Pieken et al. (1991) Science 253:314-7). For instance, the modified nucleotide could be substituted with amino and fluoro functional groups at the 2′ position. In addition, further functional moieties of interest include, 2′-O-methyl purine nucleotides and phosphorothioate modified nucleotides (Green et al. (1995) Chem. Biol. 2:683-695; Vester et al. (2002) J. Am. Chem. Soc. 124:13682-13683; Rhodes et al. (2000) J. Biol. Chem. 37:28555-28561; and, Seyler et al. (1996) Biol. Chem. 377:67-70). Accordingly, in another embodiment, the surrogate antibody molecules comprise functional moieties comprising modified nucleotides that stabilize the molecule in the presence of serum nucleases.

Other modifications of interest include chemical modifications to one or more nucleotides in the specificity domain of the specificity strand, wherein the modified nucleotide introduces hydrophobic binding capabilities into the specificity domain. In certain embodiments, this chemical modification occurs at the 2′ position of the nucleotide sugar or phosphate molecule. Such modifications are known in the art and include for example, non-polar, non-hydrogen binding shape mimics such as 6-methyl purine and 2,4-difluorotolune (Schweizer et al. (1995) J Am Chem Soc 117:1863-72 and Guckian et al. (1998) Nat Struct Biol 5:950-9, both of which are herein incorporated by reference). Additional modifications include the addition of imizadole, phenyl, proline, and isoleucyl.

In other embodiments, it is desirable to preferentially amplify the specificity strand of the surrogate antibody molecule. By “preferentially amplify” is intended that the specificity strand of the surrogate antibody molecule is amplified during the amplification step at an elevated frequency as compared to the amplification level of the corresponding stabilization strand. Accordingly, modifications of interest include those that allow for the preferential amplification of the specificity strand of the surrogate antibody molecule. While methods of amplifying the surrogate antibodies are discussed in further detail elsewhere herein, the type of modification that would allow this type of amplification are known in the art, and include, for example, a modification of at least one nucleotide on the stabilization strand that increases resistance to polymerase activity in a PCR reaction. Such modifications include any functional moiety that disrupts amplification including, for example, biotin.

Additional modifications of interest include, for example, attachment of a detectable label. As used herein a “detectable label” refers to a molecule that permits of the detection of the surrogate antibody that it is attached to. Accordingly, in another embodiment, the incorporation or attachment of a detectable label as a functional moiety permits detection of the surrogate antibody and the complexed target ligand. Such detectable labels include, for example, a polypeptide; radionucleotides (e.g. ³²P); fluorescent molecules (Jhaveri et al. (2000) J. Am. Chem. Soc. 122:2469-2473, luminescent molecules, and chromophores (such as FITC, Fluorescein, TRITC, Methyl Umbiliferone, luminol, luciferin, and Texas Red (Sumedha et al. (1999) Clinical Chemistry 45:1628-1649, Wilson et al. (1998) Clin Chemistry 44:86-91, and Henegariu (2000) Nature Biotechnology 18:345-349); enzymes (e.g. horseradish peroxidase, alkaline phosphatase, urease, β-Galactosidase, peroxidase, proteases, etc.), lanthanide series elements (e.g. europium, terbium, yttrium), and microspheres (e.g. sub-micron polystyrene, dyed or undyed), as well as other detectable labels described elsewhere herein. Such detectable labels allow for direct qualitative or quantitative detection.

In one embodiment, the functional moiety comprising a detectable label is digoxigenin. Detection of this functional moiety is achieved by incubation with anti-digoxigenin antibodies coupled directly to several different fluorochromes or enzymes or by indirect immunofluorescence. See, Ausubel et al. Current Protocols in Molecular Biology, John Wiley & Sons, Inc. and Celeda et al. (1992) Biotechniques 12:98-102, both of which are herein incorporated by reference. Additional molecules that can act as detectable labels include biotin and polyA tails.

In another embodiment, the antibody is modified by the attachment of an affinity tag that can be used to attach surrogate antibodies to a solid support or to other molecules in solution. Thus, the isolation of the ligand-bound surrogate antibody complexes can be facilitated through the use of affinity tags coupled to the surrogate antibody. As used herein, an affinity tag is any compound that can be attached to a surrogate antibody molecule and be used to separate surrogate antibodies having the affinity tag from molecules that do not have the affinity tag or be used to attach compounds to the surrogate antibody. Preferably, an affinity tag is a compound that binds to or interacts with another compound, such as a ligand-binding molecule or an antibody. It is also preferred that such interactions between the affinity tag and the capturing component be a specific interaction. For example, when attaching surrogate antibody molecules to a column, microplate well, or tube containing immobilized streptavidin, surrogate antibody molecules prepared using biotinylated primers result in their binding to the streptavidin bound to the solid phase. Other affinity tags used in this manner can include a polyA sequence, protein A, receptors, antibody molecules, chelating agents, nucleotide sequences recognized by anti-sense sequences, cyclodextrin, and lectins. Additional affinity tags have been described by Syvanen et al. (1986) Nucleic Acids Res. 14:5037. Preferred affinity tags include biotin, which can be incorporated into nucleic acid sequences (Langer et al. (1981) Proc. Natl. Acad. Sci. USA 78:6633) and captured using streptavadin or biotin-specific antibodies. A preferred hapten for use as an affinity tag is digoxygenin (Kerkhof (1992) Anal. Biochem. 205:359-364). Many compounds for which a specific antibody is known or for which a specific antibody can be generated can be used as affinity tags. Antibodies useful as affinity tags can be obtained commercially or produced using well-established methods. See, for example, Johnston et al. (1987) Immunochemistry In Practice (Blackwell Scientific Publications, Oxford, England) 30-85.

Other affinity tags are anti-antibody antibodies. Such anti-antibody antibodies and their use are well known. For example, anti-antibody antibodies that are specific for antibodies of a certain class or isotype or sub-class (for example, IgG, IgM), or antibodies of a certain species (for example, anti-rabbit antibodies) are commonly used to detect or bind other groups of antibodies. Thus, one can have an antibody to the affinity tag and then this antibody:affinity tag:surrogate activity complex can then be purified by binding to an antibody to the antibody portion of the complex.

Other affinity tags include those that can form selectable cleavable covalent bonds with other molecules of choice. For example, such affinity tags include those containing a sulfur atom. A nucleic acid molecule that is associated with this affinity tag can be purified by retention on a thiopropyl sepharose column. The column may be washed to remove unbound molecules and then reduced with α-mercaptoethanol, to allow the desired molecules to be collected after purification under relatively gentle conditions.

In yet other embodiments, the functional moiety is incorporated into the specificity strand to expand the genetic code. Such moieties include, for example, IsoG/IsoC pairs and 2,6-diaminopyrimide/xanthine base pairs (Piccirilli et al (1990) Nature 343:537-9 and Tor et al (1993) J Am Chem Soc 115:4461-7); methyliso C and (6-aminohexyl)isoG base pairs (Latham et al. (1994) Nucleic Acid Research 22:2817-22), benzoyl groups (Dewey et al. (1995) J Am Chem Soc 117:8474-5 and Eaton et al. (1997) Curr Opin Chem Biol 1:10-6) and amino acid side chains.

Other functional moieties of interest include a linking molecule (i.e., iodine or bromide for either photo or chemical crosslinking; a —SH for chemical crosslinking); a therapeutic agent (i.e., compounds used in the treatment of cancer, arthritis, septicemia, myocardial arrhythmia's and infarctions, viral and bacterial infections, autoimmune and prion diseases); a chemical modification that alters biodistribution, pharmacolinetics and tissue penetration, or any combination thereof. Such modifications can be at the C-5 position of the pyrimidine residues.

Functional moieties incorporated into the surrogate antibody (either in the stabilization strand or the specificity strand or both) may be multi-functional (i.e., the moiety could allow for labeling and affinity delivery, nuclease stabilization and/or produce the desired multi-therapeutic or toxicity effects. These modified surrogate antibodies of the invention find use, for example, in aiding detection for applications such as fluorescence-activated cell sorting (Charlton et al. (1997) Biochemistry 36: 3018-3026 and Davis et al. (1996) Nucleic Acid Research 24:702-703), enzyme-linked oligonucleotide assays (Drolet et al. (1996) Nat. Biotech 14:1021-1025), and other diagnostic assays, some of which are discussed elsewhere herein.

In addition, aptamers known to bind, for example, cellulose (Yang et al. (1998) Proc. Natl. Acad. Sci. 95: 5462-5467) or Sephadex (Srisawat et al. (2001) Nucleic Acid Research 29) have been identified. These aptamers could be attached to the surrogate antibody and used as a means to isolate or detect the surrogate antibody molecules.

Various methods for attaching the functional moiety to the surrogate antibody structure are known in the art. For example, bioconjugation reactions that provide for the conjugation of polypeptides or various other compounds of interest to the surrogate antibody can be found, for example, in Aslam et al. (1999) Protein Coupling Techniques for Biomed Sciences, Macmillan Press and Solulink Bioconjugation systems at www.solulink.com, Sebestyen et al. (1998) Nature Biotechnology 16:80-85; Soukchareum et al. (1995) Bioconjugate chem. 6:43-54; Lemaitre et al. (1987) Proc. Natl Acad Sci USA 84:648-52 and Wong et al. (2000) Chemistry of Protein Conjugation and Cross-Linking, CRC, all of which are herein incorporated by reference.

A functional moiety can be attached to any region of the specificity stand or the stabilization strand or any combination thereof. In one embodiment, the functional moiety is attached to one or more of the constant domains and/or stabilization domains. In other embodiments, the functional moiety is attached to the specificity domain. One of skill in the art will recognized that site of attachment of the functional moiety will depend on the desired functional moiety, and that the functional moiety will be attached in such a away that it does not prevent the binding the surrogate antibody molecule to its target ligand.

The functional moiety(ies) chosen to incorporate into the surrogate antibody structure can be selected depending on the environmental conditions in which the surrogate antibody will be contacted with its ligand or potential ligand. For example, generating surrogate antibody libraries containing molecules having ionizable groups may provide surrogate antibodies that are sensitive to salt, and the presence of metal chelating groups may lead to surrogate antibodies that are sensitive to specific metal ions. See, for example, Lin et al. (1994) Nucleic Acids Res 22:5229-34 and Lin et al. (1995) Proc Natl Acad Sci USA 92:11044-8.

In any of the various methods and compositions described herein, various functional moieties can be conjugated onto one or more strands that form the antibodies, in one or more positions on the strands. The strands of the surrogate antibody molecule can be covalently linked to one or more, or three or more, different types of moieties. The functional moiety can be at either or both of the terminal ends of either the stabilization strand or the specificity strand, added to individual residues anywhere in either strand, attached to all or a portion of the nucleotide (i.e., pyrimidines or purines), or attached to all or a portions of a given type of nucleotide (i.e., A, G, C, T/U)) and/or attached to any region of the residue (i.e., sugar, phosphate, or nitrogenous base).

II. Arrays

The present invention provides compositions and methods useful for detecting ligands of interest in a sample. The compositions of the invention include arrays for detection, identification, and quantification of ligands of interest. The arrays rely on the use of a population of surrogate antibodies that bind to ligands of interest in a sample to form a binding partner complex. The binding partner complex is immobilized onto a solid support to allow for the detection, identification, and/or quantification of the ligand of interest. By “population of surrogate antibodies”, it is intended a group or collection that comprises at least two, at least three, at least four, at least five, at least seven, at least 10, at least 100, at least 1,000, at least 10,000, at least 1×10⁶, at least 1×10⁷, or at least 1×10⁸ surrogate antibodies. Populations of surrogate antibodies include, for example, a library of surrogate antibodies, comprising a population of surrogate antibodies having a randomized specificity region. In some embodiments, the members of the population of surrogate antibodies are found in a mixture, while in other embodiments the members of the population can be attached to discrete locations on an array of separated by some other means e.g., in separate wells of a multi-well plate). In some embodiments, the ligand binding specificity of the surrogate antibodies in the population of surrogate antibodies is unknown, while in other embodiments, one or more surrogate antibodies in the population may be selected based on their ability to bind a particular ligand of interest. Methods for selecting for a surrogate antibody that binds to a particular ligand of interest are provided elsewhere herein.

In some embodiments of the invention, the arrays comprise a population of capture probes attached to discrete, known locations on a solid support or substrate. The capture probes comprise capture nucleotide sequences that are capable of binding to a surrogate antibody molecule of the invention via an interaction with a recognition nucleotide sequence comprised in the oligonucleotide tail of surrogate antibody molecule. In further embodiments, the arrays of the invention further comprise one or more surrogate antibodies that are bound to the capture probes by means of an interaction between the recognition nucleotide sequence found in the oligonucleotide tail of the surrogate antibody and the capture nucleotide sequence found in the corresponding capture probe.

In other embodiments of the invention, the surrogate antibody is attached directly to the solid support without the use of a capture probe to create the array. Methods of attaching nucleic acid molecules to a solid support are well know to those of skill in the art and are described elsewhere herein. When the surrogate antibodies are attached directly to the solid support without the use of a capture probe, the surrogate antibody need not comprise one oligonucleotide tail comprising a recognition nucleotide sequence.

A. Solid Supports

The arrays of the invention comprise a population of capture probes attached to discrete, known locations on a solid support or substrate. As used herein, “solid support” is defined as any surface to which molecules may be attached through either covalent or non-covalent bonds. This includes, but is not limited to, membranes, microsphere particles, such as Lumavidin™ or LS-beads, microtiter plates, magnetic beads, charged paper, nylon, Langmuir-Bodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is also contemplated. This includes surfaces with any topology, including, but not limited to, spherical surfaces and grooved surfaces.

The solid support or substrate of the invention may also be an organic polymer. As used herein, the term “organic polymer” is intended to mean a support material which is most preferably chemically inert under conditions appropriate for biopolymer synthesis and which comprises a backbone comprising various elemental substituents including, but not limited to, hydrogen, carbon, oxygen, fluorine, chlorine, bromine, sulfur and nitrogen. Representative polymers include, but are not limited to, the following: polypropylene, polyethylene, polybutylene, polyisobutylene, polybutadiene, polyisoprene, polyvinylpyrrolidone, polytetrafluoroethylene, polyvinylidene difluoride, polyfluoroethylene-propylene, polyethylene-vinyl alcohol, polymethylpentene, polychlorotrifluoroethylene, polysulfones, and blends and copolymers thereof.

Although a planar array surface is preferred, the array may be fabricated on a solid support of virtually any shape or even a multiplicity of surfaces. Arrays may be nucleic acids on beads, gels, polymeric surfaces, fibers such as fiber optics, glass or any other appropriate substrate. See, U.S. Pat. Nos. 5,770,358, 5,789,162, 5,708,153, 5,800,992, and 6,040,193, each of which is hereby incorporated in its entirety.

The arrays of the invention comprise a solid support having a plurality of discrete locations or addresses, where capture probes or surrogate antibodies are immobilized at the addresses. The arrays may be low-density arrays or high-density arrays and may contain 4 or more, 8 or more, 12 or more, 16 or more, 20 or more, 24 or more, 32 or more, 48 or more, 64 or more, 72 or more 80 or more, 96, or more addresses, or 192 or more, 288 or more, 384 or more, 768 or more, 1536 or more, 3072 or more, 6144 or more, 9216 or more, 12288 or more, 15360 or more, or 18432 or more addresses. In some embodiments, the substrate has no more than 12, 24, 48, 96, or 192, or 384 addresses, no more than 500, 600, 700, 800, or 900 addresses, or no more than 1000, 1200, 1600, 2400, or 3600 addressees.

The area of surface of the substrate covered by each of the address is preferably no more than about 0.25 mm². Preferably, the area of the substrate surface covered by each of the addresses is between about 1 μm² and about 10,000 μm². For example, each address may cover an area of the substrate surface from about 100 μm² to about 2,500 μm². In an alternative embodiment, an address on the array may cover an area of the substrate surface as small as about 2,500 nm².

The addresses of the array may be of any geometric shape. For instance, the addresses may be rectangular or circular. The addresses of the array may also be irregularly shaped. The distance separating the addresses of the array can vary. For example, the patches of the array are separated from neighboring patches by about 1 μm to about 500 μm. Typically, the distance separating the patches is roughly proportional to the diameter or side length of the addresses on the array if the addresses have dimensions greater than about 10 μm. If the address size is smaller, then the distance separating the patches will typically be larger than the dimensions of the patch.

Typically, only one type of capture is present on a single address of the array. If more than one type of capture probe is present on a single address, all of the capture probes must interact with a surrogate antibodies that share a common binding partner.

The array formats of the present invention may be included in a variety of different types of devices. The term “device” is intended to mean any device to which the solid support can be affixed, such as microtiter plates, test tubes, inorganic sheets, dipsticks, etc. Any device may be used, so long as the solid support can be affixed thereto without affecting the functional behavior of the solid support or any biopolymer adsorbed thereon, and that the device is stable to any materials into which the device is introduced (e.g., clinical samples, etc.).

B. Capture Probes

In some embodiments the arrays of the invention comprise a plurality of capture probes that are immobilized onto the solid support to create the array. The capture probes are immobilized onto the solid support a discrete locations or “addresses.” The capture probes comprise a known “capture nucleotide sequence” that is capable of interacting with the recognition nucleotide sequence of a corresponding surrogate antibody. Typically, the sequence of the capture nucleotide sequence attached to each address is known. The capture probes may comprise additional nucleotide sequences that serve as spacers or as linkers for attachment to the solid support.

The array typically comprises different types of capture probes. By “different types” of capture probes, it is intended capture probes having different capture nucleotide sequences, i.e. capture nucleotide sequences that vary by one or more nucleotides. In some embodiments, the array comprises at least two or at least five different types of capture probes. In other embodiments, the array comprises at least 10, at least 20, at least 30, at least 50, or at least 80 different types of capture probes. In still other embodiments, the array may comprise at least 100, at least 1000, at least 10,000, or at least 50,000 different types of capture probes. The number of addresses of the array may vary with the purpose for which the array is intended. For instance, if the array is to be used as a diagnostic tool in evaluating the status of a tumor or other disease state in a patient, an array comprising less than about 100, less than about 60, less than about 30, less than about 15, or less than about 10 different addresses may suffice since the necessary binding partner complexes of the capture probes on the array are limited to only those proteins whose expression is known to be indicative of the disease condition. However, if the array is to be used to measure a significant portion of the total protein content of a cell, then the array may comprise at least about 1,000 or at least about 10,000 different types of capture probes.

In one embodiment of the array, each of the addresses of the array comprises a different type of capture probe. For instance, an array having 100 addresses could comprise about 100 different types of capture probes. Likewise, an array having about 10,000 addresses could comprise about 10,000 different capture probes. In an alternative embodiment, each different type of capture probe is immobilized on more than one separate address on the array. For instance, each different protein-capture agent may optionally be present on at two, three, four, five, six or more different addresses. An array of the invention, therefore, may comprise about three thousand different addresses, but only comprise about one thousand different types of capture probes, since each different type of capture probe is present on three discrete addresses. Such a format may be useful for increasing the precision of measurements for quantifying the ligand of interest. The use of replicate addresses is described by Yang et al. (2002) Nucleic Acids Res. 30:e15, and reviewed by Churchill (2002) Nature Genetics Supplement 32:490-95 and Quackenbush (2002) Nature Genetics Supplement 32:496-501; each of which is hereby incorporated in its entirety by reference.

The capture nucleotide sequences comprised in the capture probes of the invention can be of any length so long as they hybridize to the recognition nucleotide sequence of a corresponding surrogate antibody. For any given capture nucleotide sequence, an optimum length for use with a particular recognition nucleotide sequence under specified screening conditions can be determined empirically. Thus, the length and composition of each capture nucleotide sequence comprised in the array may be optimized for the screening of particular target materials under specific conditions (for example, at a given temperature, pH, osmolarity, or solvent). The length of the capture probe can be at least 8, at least 10, at least 12, at least 15, at least 18, at least 20, at least 25, at least 30, at least 40, at least 50, at least 60, at least 70, at least 80, at least 90, at least 100, at least 150, at least 200, at least 250, at least 300, at least 400, at least 500, at least 600, at least 700, at least 800, at least 1000, at least 2000, at least 4000, or at least 8000 nucleotides in length. For example, the capture probe can be about 10-15, about 15-20, about 20-25, about 25-35, about 35-50, about 50-75, about 75-100, about 100-150, about 150-300, about 300-600, about 600-1000, about 1000-1500, about 1500-2500, or about 2500-5000 nucleotides in length.

C. Synthesis of Arrays

Arrays, also described as “microarrays” or colloquially as “chips,” and methods for generating arrays comprising known nucleotide sequences at addressable (discrete and known locations) locations have been generally described in the art. See, for example U.S. Pat. Nos. 5,143,854, 5,445,934, 5,744,305, 5,677,195, 6,040,193, 5,424,186, 6,329,143, and 6,309,831, and Fodor et al. (1991) Science 251:767-77, each of which is incorporated by reference in its entirety. These arrays may generally be produced using mechanical synthesis methods or light directed synthesis methods, which incorporate a combination of photolithographic methods and solid phase synthesis methods. In some embodiments of the present invention, the capture probes are synthesized separately and then attached to the solid support to create the array. In other embodiments, the sequences of the capture probes are synthesized directly on the support to create the desired array. Suitable methods for covalently coupling oligonucleotides to a solid support and for directly synthesizing the oligonucleotides are known to those in the art. A summary of suitable methods is found, for example, in Matson et al. (1994) Analytical Biochem. 217: 306-310, herein incorporated by reference. See, also, PCT applications WO 85/01051 and WO 89/10977 and U.S. Pat. Nos. 5,384,261 5,429,806, 5,981,185, and 6,492,118, each of which is incorporated herein by reference.

D. Immobilization of Surrogate Antibodies on the Array

In some embodiments of the invention the surrogate antibodies of the invention are immobilized on the arrays of the invention by means of an interaction between a recognition nucleotide sequence comprised within an oligonucleotide tail of the surrogate antibody and a capture nucleotide sequence comprised within the corresponding capture probe of the array. In further embodiments, the population of surrogate antibodies is immobilized on the array prior to being contacted with the population of test ligands. In other embodiments, the population of surrogate antibodies is contacted with the population of test ligands to allow the formation of binding partner complexes prior to being immobilized on the array. In still other embodiments, the surrogate antibodies are immobilized on the array in the presence of the population of test ligands.

The population of surrogate antibodies and the array comprising the capture probes may be brought into contact under conditions that allow the hybridization of the recognition nucleotide sequence comprised in the oligonucleotide tail of the surrogate antibody and the capture nucleotide sequence comprised in the capture probe. The conditions conducive to hybridization will vary with the recognition nucleotide sequence and the capture nucleotide sequence due to the unique melting temperatures and hybridization properties of different polynucleotides. Melting temperature (Tₘ) is determined largely by the length of the region of complementarity, the number of mismatched base pairs in the region of complementarity, the number of hybridizing guanine-cytosine base pairs in the hybrid, and the composition and temperature of the solution in which the hybridization step is performed. Generally, lower temperature and higher ionic strengths favor hybridization. However, higher temperatures and lower ionic strengths can be used to increase specificity at the expense of decreased sensitivity, because these conditions destabilize nonspecific hybrids. The effects of base composition on duplex stability may be reduced by carrying out the hybridization in particular solutions, for example in the presence of high concentrations of tertiary or quaternary amines. By carrying out the hybridization at temperatures close to the anticipated Tₘ's of the type of duplexes expected to be formed between the capture probes and the oligonucleotides tails of the surrogate antibody, the rate of formation of mismatched duplexes may be substantially reduced.

A chaotropic hybridization solvent, such as a ternary or quaternary amine may also be used. In this regard, tetramethylammonium chloride (TMACl) at concentrations in the range of about 2 M to about 5.5 M is particularly suitable; at TMACl concentrations around 3.5 to 4 M, the Tₘ dependence on nucleotide composition is substantially reduced.

In addition, the choice of hybridization salt has a major effect on overall hybridization yield; for example, TMACl at concentrations up to 5 M can increase the overall hybridization yield by a factor of up to 30 or more (depending to some extent on the nucleotide composition) compared to 1 M NaCl. Finally, as previously noted, the length of the oligonucleotides attached to the array may be varied so as to optimize hybridization under the particular conditions employed. Thus, the hybridization conditions are generally those that permit discrimination between exactly matched and mismatched oligonucleotides.

Preferred hybridization conditions will maintain the stability of binding partner complexes formed between the surrogate antibodies of the invention and the compounds or ligands of interest. Surrogate antibody molecules that bind to a ligand of interest under conditions conducive to the hybridization of the recognition nucleotide sequences and the capture nucleotide sequence may be produced using methods described elsewhere herein. Thus, in some embodiments the conditions used for hybridization will be those used to select for a surrogate antibody that binds to the ligand of interest.

Generally, the concentration of capture probe should be sufficient relative to the concentration of the surrogate antibody to produce detectable hybridization between the capture probe and the surrogate antibody where such hybridization is appropriate, for example, by using a molar excess of capture probe.

III. Kits

The present invention provides kits comprising an array of the invention. These kits are useful in the methods of detection, methods of quantification, and methods of screening described elsewhere herein. The kits may also be designed for use in a method of identifying molecules that present at different levels in two or more samples. In other embodiments, the kits are designed for the identification of particular types of samples and contain surrogate antibodies that bind to ligands that are present at different levels in two or more samples.

In some embodiments the kits comprise arrays having a population of capture probes attached to discrete, known locations on a solid support or substrate, with one or more surrogate antibodies molecules of the invention immobilized to the array by means of an interaction between a recognition nucleotide sequence found in the oligonucleotide tail of the surrogate antibody and a capture nucleotide sequence found in the corresponding capture probe. In other embodiments, kits comprise an array having capture probes attached to discrete, known locations on a solid support or substrate, where the capture probes comprise capture nucleotide sequences that are capable of binding to a surrogate antibody molecule of the invention by means of an interaction with a recognition nucleotide sequence comprised in the oligonucleotide tail of surrogate antibody molecule. In some embodiments of the kit, the population of surrogate antibodies is preferably provided as a separate kit component. The kit may additionally comprise secondary molecules for use in detection of binding partner complexes. The population of surrogate antibodies and the population of secondary molecules may be provided in solution, or they may be provided as a solid phase (e.g., lyophilized).

Additional compositions may be included in a kit of the invention. Such compositions include one or more buffers for use in contacting the test compounds with the population of surrogate antibody molecules to allow the formation of a binding partner complex between a test compound and a surrogate antibody. The kit may also include instructions for use in a method of detection or quantification of ligands of interest.

In some embodiments, a kit of the invention includes a computer-readable medium comprising one or more digitally-encoded reference ligand profiles, where each reference profile has one or more values representing the level of a ligand that is detected by an array of the invention. These kits are useful for determining whether a test sample is of the same sample type as the reference samples using methods described elsewhere herein.

### Methods

I. Methods of Detecting a Ligand of Interest

The present invention provides methods for detecting one or more ligands of interest in a population of test ligands. In one embodiment, the methods comprise the steps of

1) contacting the population of test ligands with a population of surrogate antibody molecules under conditions that allow for the formation of a binding partner complex between at least one of the surrogate antibody molecules and at least one ligand of interest, where the surrogate antibody molecule comprises
a) a specificity strand having a specificity domain flanked by a first constant domain and a second constant domain;
b) a stabilization strand comprising a first stabilization domain that interacts with said first constant domain and a second stabilization domain that interacts with said second constant domain; and,
c) at least one oligonucleotide tail comprising a recognition nucleotide sequence that is unique to the particular surrogate antibody molecule;

2) forming at least one binding partner complex;

3) providing an array comprising a population of capture probes attached to a solid support, where the capture probes are attached to a discrete know region of the solid support and comprise a capture nucleotide sequence that is complementary to at least one recognition nucleotide sequence;

4) contacting the binding partner complex with the array under conditions that allow for the hybridization of the recognition nucleotide sequence of the surrogate antibody with the capture nucleotide sequence of the corresponding capture probe; and

5) detecting the binding partner complex bound to the array to thereby detect the ligand of interest.

In another embodiment, the method for detecting a ligand of interest in a population of test ligands comprises the steps of

1) providing an array having a population of capture probes, where the capture probes are attached to discrete, known locations on a solid support, the capture probes comprise a known capture nucleotide sequence, and a population of surrogate antibody molecules are bound to the capture probes by an interaction between the capture nucleotide sequence and a recognition nucleotide sequence comprised within an oligonucleotide tail of the surrogate antibody, where the surrogate antibody molecules further comprise:
a) a specificity strand having a specificity domain flanked by a first constant domain and a second constant domain; and
b) a stabilization strand comprising a first stabilization domain that interacts with said first constant domain and a second stabilization domain that interacts with said second constant domain;

2) contacting a population of test ligands with the array under conditions that allow for the formation of a binding partner complex between at least one of the surrogate antibody molecules bound to the array and at least one of ligand of interest; and

3) detecting the binding partner complex.

A. Contacting the Population of Test ligands with the Surrogate Antibodies

The invention provides methods for detection, identification, and/or quantification of one or more ligands of interest. In the methods, a population of test ligands is contacted with a population of surrogate antibody molecules under conditions that allow for the formation of a binding partner complex between at least one of the test ligands and at least one of the surrogate antibodies. In some embodiments of the present invention, the population of surrogate antibodies is immobilized on an array prior to being contacted with the population of test ligands. The population of test ligands is then contacted with the array under conditions that promote the formation of a specific binding partner complex between one of more surrogate antibodies on the array and the corresponding ligand of interest in the population of test ligands.

In other embodiments, the population of test ligands is contacted with the population of surrogate antibodies and binding partner complexes are formed before the population of surrogate antibodies is contacted with the array. In some embodiments, the population of test ligands and the binding partner complexes are provided in a liquid. In other embodiments, the population of surrogate antibodies is provided as a solid phase, and the population of test ligands is added to the population of surrogate antibodies under conditions that promote the formation of one or more binding partner complexes. For example, the surrogate antibodies may be dried or lyophilized (i.e., prepared by rapid freezing and drying in a vacuum) prior to being contacted with the population of test ligands. The population of test ligands is then added to the surrogate antibodies under conditions that promote the formation of a binding partner complex between at least one surrogate antibody and a corresponding ligand of interest. The binding partner complexes are then contacted with an array of capture probes under conditions that allow the interaction of the recognition nucleotide sequence comprised in the oligonucleotide tail of the surrogate antibody to interact with the capture nucleotide sequence comprised in the corresponding capture probe. The array will preferably be contacted under conditions that maintain the stability of the interaction between the surrogate antibody and the test ligand in the binding partner complex. Interaction between the recognition nucleotide sequence and the corresponding capture nucleotide sequence immobilizes the binding partner complex at a discrete location or address on the array.

In still other embodiments, the population of test ligands is contacted with the population of surrogate antibodies in the presence of the array of capture probes. The population of test ligands is contacted with the population of surrogate antibodies under conditions that promote the formation of a binding partner complex between at least one surrogate antibody and a corresponding ligand of interest. Preferably, the conditions will also allow the interaction of the recognition nucleotide sequence comprised within the oligonucleotide tail of the surrogate antibody to interact with the capture nucleotide sequence of a corresponding capture probe on the array. Interaction between the recognition nucleotide sequence and the corresponding capture nucleotide sequence immobilizes the binding partner complex at a discrete location (address) on the array.

The population of test ligands is contacted with the population of surrogate antibodies for a period of time sufficient to allow the formation of a binding partner complex between a surrogate antibody and a ligand of interest. Typically, population of test ligands is contacted with the population of surrogate antibodies for a period of between about 30 seconds and about 2 hours. In some embodiments, the population of test ligands is contacted to the population of surrogate antibodies for a period of between about 60 seconds and about 30 minutes.

The temperature at which the population of test ligands is contacted with the extract is a function of the particular test ligands and surrogate antibodies selected. Typically, the test ligand is contacted with the surrogate antibody under physiologic temperature conditions, however, for some samples, modified temperature (typically 4° C. to 50° C.) can be desirable and will be empirically determinable by those skilled in the art.

One advantage of the present invention over conventional detection techniques is that the present invention enables the detection of numerous different ligands of interest to be conducted using only very small amount of sample. Generally, a volume of sample containing from about 5 to about 200 μl is sufficient to allow for detection of the ligand of interest.

In some embodiments, the binding partner complex will be detected under homogenous reaction conditions, such that it will not be necessary to remove unbound test ligands (i.e. test ligands that are not bound by a surrogate antibody) or unbound surrogate antibodies (i.e. surrogate antibodies that are not bound to a ligand of interest) from the binding partner complex prior to detection of the binding partner complex. In other embodiments of the invention, it is preferred to remove unbound test ligands, unbound surrogate antibodies, or both unbound test ligands and unbound surrogate antibodies from the mixture containing the binding partner complex prior to detecting the binding partner complex. For example, where detection of the binding partner complex is accomplished by labeling the population of test ligands, it may be necessary to remove unbound test ligands prior to the detection step.

Any method known in the art may be used to remove the unbound test ligands or unbound surrogate antibodies from the binding partner complex. For example, in some embodiments, unbound test ligands are removed from the binding complex by washing the array on which the binding partner complex has been immobilized. The conditions for the wash step are designed to maintain the stability of specific binding partner complexes and the stability of the interaction between the recognition nucleotide sequence of the surrogate antibody with the capture nucleotide sequence of the corresponding capture probe, while removing unbound test ligand from the array. In other embodiments, where the binding partner complex prior to immobilization of the surrogate antibodies on the array, the unbound test ligands may be removed from the binding partner complex by partitioning, using methods described elsewhere herein. After the partitioning step, the binding partner complex is contacted with an array of the invention to allow detection.

B. Detection of Binding Partner Complexes

After the binding partner complexes are immobilized to the array of the invention, the complexes may be detected and quantitated by measuring a complex-dependent signal associated with discrete locations on the array. A number of detection methods may be used in the present invention to produce a complex-dependent signal, and the detection step may be either be qualitative (i.e., for purposes of detection only) or quantitative (i.e., the amount of binding complex immobilized on the array may be measured). Methods for the detection of molecules immobilized on an array are known in the art. Examples of non-label detection methods include those based on optical waveguides, surface plasmon resonance, surface charge sensors, and surface force sensors are compatible with many embodiments of the invention. See, for example, PCT Publication WO 96/26432 and U.S. Pat. No. 5,677,196 both of which are herein incorporated by reference in their entirety. Alternatively, technologies such as those based on Brewster Angle microscopy (BAM) and ellipsometry could be applied. See, for example, Schaaf et al. (1987) Langmuir 3:1131-1135; U.S. Pat. Nos. 5,141,311 and 5,116,121; and Kim (1984) Macromolecules 22:2682-2685; each of which is herein incorporated by reference in its entirety. Quartz crystal microbalances and desorption processes provide still other alternative detection means suitable for at least some embodiments of the invention array. See, for example, U.S. Pat. No. 5,719,060, herein incorporated by reference. An example of an optical biosensor system compatible both with some arrays of the present invention and a variety of non-label detection principles including surface plasmon resonance, total internal reflection fluorescence (TIRF), Brewster Angle microscopy, optical waveguide lightmode spectroscopy (OWLS), surface charge measurements, and ellipsometry can be found in U.S. Pat. No. 5,313,264.

Detection can be facilitated by coupling (i.e., physically linking) the test ligand, the surrogate antibody, or both the test ligand and the surrogate antibody to a detectable label. The detectable label typically generates a measurable signal, such as a florescent, chromogenic, or radioactive signal, that can be used to detect and quantitate the amount of binding partner complex bound to the array. Examples of detection methods for arrays based on the use of a detectable label are well known in the art. See, for example, U.S. Pat. Nos. 6,215,894, 6,329,661, 6,362,004, 6,399,35, 6,406,849, 6,447,723, and 6,471,916, each of which is herein incorporated by reference. Such methods include, but are not limited to, absorption in the visible or infrared range; chemiluminescence; and fluorescence, including lifetime fluorescence, polarization, fluorescent quenching, fluorescence correlation spectroscopy (FCS), and fluorescence-resonance energy transfer (FRET)). The use of detection methods such as fluorescent quenching and FRET allow for the detection to be performed under homogeneous reactions conditions such that is not necessary to remove unbound labeled compounds from the array prior to the detection step. Such methods typically rely on the use of a fluorescent group that, when excited with light having a selected wavelength, emits light of a different wavelength, and a fluorescence-modifying group that can modify the fluorescent signal of the fluorescent group. The fluorescent group is attached to one component of the binding complex, while the fluorescence-modifying group is attached to another component of the binding partner complex. When the binding partner complex is formed, the fluorescent group is brought into close physical proximity with the fluorescence-modifying group, resulting in a corresponding change in the detectable fluorescent signal. See, for example U.S. Pat. No. 6,177,555, herein incorporated in its entirety by reference.

The selection of the detection method will depend upon the labeling group used. Examples of fluorescent and luminescent detectable labels include, but are not limited to, fluorescein, tetramethylrhodamine, Texas Red, BODIPY, 5-[(2-aminoethyl)amino] napthalene-1-sulfonic acid (EDANS), FITC, TRITC, isothiocyanate, rhodamine, dichlorotriazinylamine, dansyl chloride, phycoerythrin umbiliferone, luminol, aequorin, and luciferin. Non-limiting examples of enzyme-based detectable labels include horseradish peroxidase and other peroxidases, alkaline phosphatase, acetylcholinesterase, urease, β-Galactosidase, and proteases. For example, inactive β-galactosidase monomers and an inducer peptide may be conjugated to a ligand of interest, resulting in the formation of active β-galatosidase tetramer and substrate conversion. The addition of surrogate antibody specific for the ligand of interest will then interfere with β-galactosidase polymerization and substrate conversion. Examples of suitable radioactive detectable labels include, but are not limited to ³²P, ¹²⁵I, ¹³¹I, ³⁵S or ³H.

In some embodiments, the labeling group is linked to the population of test ligands. After one or more binding partner complexes are formed between the ligand of interest and a surrogate antibody, the unbound test ligand is removed by partitioning the binding partner complex from the unbound or non-specifically bound test ligands, or by washing the array comprising the binding partner complex to remove the unbound test ligand. Methods for partitioning the binding partner complex from unbound or non-specifically-bound ligands are described elsewhere herein. The binding partner complex may then be detected by assaying for the signal produced by the detectable label. In some embodiments, the binding partner for the surrogate antibody which interacts with a particular capture probe on the array is known, thereby allowing the identification of a particular ligand of interest by detecting the complex bound to a particular address on the array.

In other embodiments, the binding partner complex is detected indirectly using a secondary molecule. In this method, the secondary molecule contains a detectable label, and the binding partner complex is detected using a two-site binding or sandwich-type assay. Typically, detection using a sandwich assays is based on the specific binding of a labeled secondary molecule to a target molecule or target complex that has been immobilized on a solid support. The unbound secondary molecules are removed (e.g., by washing) and then the signal from the detectable label on the secondary molecule is measured, thereby allowing for the detection and quantification of the target molecule or target complex bound by the secondary molecule. See, for example, U.S. Patent Application Number 20020037506, herein incorporated by reference.

Accordingly, the present invention provides a method for detecting the presence of a ligand of interest in a population of test ligands, where the method comprises the following steps: (1) contacting a population of test ligands with a population of surrogate antibodies of the invention under conditions in which a binding partner complex is formed between at least one ligand of interest and a surrogate antibody, where the surrogate antibodies are immobilized on an array of the invention by means of an interaction between a recognition nucleotide sequence comprised within the oligonucleotide tail of the surrogate antibody and the capture nucleotide sequence comprised within the corresponding capture probe; (2) contacting the binding partner complexes immobilized on an array with one or more secondary molecules, where the secondary molecules comprise a detectable label and are capable of specifically binding to a binding site found in the binding partner complex on either the ligand of interest or a surrogate antibody; (3) removing unbound secondary molecule; and (4) detecting the signal from the detectable label found on the secondary molecule to thereby detect the ligand of interest.

The secondary molecules used in the invention may be any molecules capable of binding to the ligand of interest or to the surrogate antibodies. Examples of secondary molecules that may be used include, but are not limited to, antibodies, surrogate antibodies (i.e. surrogate antibodies of the present invention), and nucleic acid probes. In some embodiments, the surrogate antibody or test ligand is modified to allow binding of the secondary molecule. For example, the surrogate antibody or test ligand may conjugated with biotin, and a streptavidin molecule containing a detectable label may be used as a secondary molecule. See, for example Davis et al. (1996) Nucleic Acids Res. 24:702-706. The surrogate antibody or test ligand may also be modified by the addition of any protein or moiety that is specifically recognized by a secondary molecule. See, for example, Drolet et al. (1996) Nature Biotechnol. 14:1021-1025. In other embodiments, the secondary molecule is designed or selected to bind specifically to a particular surrogate antibody or to a particular test ligand. For example, the secondary molecule may be a second surrogate antibody. Methods for selecting for surrogate antibodies that bind specially to a particular target compounds are described elsewhere herein. Where a second surrogate antibody is used as a secondary molecule for detection, it is not required that the second surrogate antibody comprise an oligonucleotide tail comprising a recognition nucleotide sequence.

It is recognized that where a secondary molecules is used for detection of the binding partner complex, the secondary molecule should be designed or selected so that it does not disrupt the formation of the binding partner complex, for example, by binding to the ligand binding domain of the surrogate antibody in a manner that prevents the binding of the ligand of interest. Accordingly, secondary molecules that recognize a site on the ligand of interest or the corresponding surrogate antibody that are distinct from the sites involved in the interaction between the test ligand and the corresponding surrogate antibody are preferred.

C. Quantitation of Ligands of Interests

The methods of the present invention allow for the quantitation of ligands of interest within a population of test ligands. The population of test ligands is contacted with a population of surrogate antibodies of the invention under conditions that allow for the formation of a binding partner complex between one or more ligands of interest and a corresponding surrogate antibody. The binding partner complex is detected using methods described elsewhere herein, resulting in a raw value corresponding to the amount of binding partner complex bound to the array. The amount of binding partner complex formed and bound to array is correlated with the level of the ligand of interest in the sample, thereby allowing quantitation of the ligand of interest.

In some embodiments, it will be preferred to normalize the values obtain by detecting the binding partner complex on the array so that results obtained from separate experiments or from different samples may be compared For example, the detection data can be normalized with reference to a “control ligand” that is present at similar levels in different populations of test ligands. In addition, a given type of capture probe may be attached to the array at more than one address on the array with the result that the corresponding binding complex will be detected at multiple discrete locations on the array. By obtaining multiple raw values corresponding to the amount of binding partner complex formed, the accuracy of detection and quantification can be increased. Methods for designing array experiments to increase the accuracy of quantitation, and methods for analyzing and normalizing array results, and for validating array results are known in the art. Such methods are reviewed, for example, in Holloway et al. (2002) Nature Genetics Suppl. 32:481-89, Churchill (2002) Nature Genetics Suppl. 32:490-95, Quackenbush (2002) Nature Genetics Suppl. 32: 496-501; Slonim (2002) Nature Genetics Suppl. 32:502-08; and Chuaqui et al. (2002) Nature Genetics Suppl. 32:509-514; each of which is herein incorporated by reference in its entirety.

II. Methods of Creating and Using Ligand Profiles

The present invention provides methods for generating a ligand profile for a sample. In one embodiment, the method comprises the steps of:

1) contacting the sample with a population of surrogate antibody molecules under conditions that allow for the formation of a binding partner complex between at least one of the surrogate antibody molecules and at least one ligand of interest in the sample, wherein the surrogate antibody molecule comprises
a) a specificity strand having a specificity domain flanked by a first constant domain and a second constant domain;
b) a stabilization strand comprising a first stabilization domain that interacts with said first constant domain and a second stabilization domain that interacts with said second constant domain; and,
c) at least one oligonucleotide tail comprising a recognition nucleotide sequence that is unique to the particular surrogate antibody molecule;

2) providing an array comprising a population of capture probes attached to a solid support, where the capture probes are attached to a discrete, known region of the solid support and comprise a capture nucleotide sequence that is complementary to at least one recognition nucleotide sequence;

3) contacting any binding partner complexes formed in step a) with the array under conditions that allow for the hybridization of the recognition nucleotide sequence of the surrogate antibody with the capture nucleotide sequence of the corresponding capture probe;

4) detecting the binding partner complex bound to the array; and

5) generating a ligand profile for the sample, wherein said ligand profile comprises values representing the level of one or more ligands that are present in the sample.

In another embodiment, the method for generating a ligand profile for a sample comprises the steps of:

1) providing an array having a population of capture probes, where the capture probes are attached to discrete, known locations on a solid support, the capture probes comprise a known capture nucleotide sequence, and a population of surrogate antibody molecules are bound to the capture probes by an interaction between the capture nucleotide sequence and a recognition nucleotide sequence comprised within an oligonucleotide tail of the surrogate antibody, where the surrogate antibody molecules further comprise:
a) a specificity strand having a specificity domain flanked by a first constant domain and a second constant domain;
b) a stabilization strand comprising a first stabilization domain that interacts with said first constant domain and a second stabilization domain that interacts with said second constant domain; and
c) wherein the oligonucleotide trail comprises a recognition nucleotide is unique to the particular surrogate antibody molecule,

2) contacting the sample with the array under conditions that allow for the formation of a binding partner complex between at least one of the surrogate antibody molecules bound to the array and at least one ligand of interest in the sample;

3) detecting the binding partner complex; and

4) generating a ligand profile for the sample, wherein said ligand profile comprises values representing the level of one or more ligands that are present in the sample.

The present also provides a method for identifying surrogate antibody ligands that are present at different levels in two or more samples. The method comprises the steps of

1) separately contacting each sample with a population of surrogate antibody molecules, wherein the surrogate antibody molecules comprise:
a) a specificity strand having a specificity domain flanked by a first constant domain and a second constant domain;
b) a stabilization strand comprising a first stabilization domain that interacts with said first constant domain and a second stabilization domain that interacts with said second constant domain; and,
c) at least one oligonucleotide tail comprising a recognition nucleotide sequence that is unique to the particular surrogate antibody molecule;

2) for each sample, forming one or more binding partner complexes between a surrogate antibody and a ligand if the sample contains a ligand that is bound by one or more surrogate antibodies in the population of antibodies;

3) for each sample, providing an array comprising a population of capture probes attached to a solid support, where the capture probes are attached to a discrete, known locations on the solid support and comprise a capture nucleotide sequence that is complementary to at least one recognition nucleotide sequence;

4) for each sample, contacting any binding partner complex formed in step b) with the array under conditions that allow for the hybridization of the recognition nucleotide sequence of the surrogate antibody with the capture nucleotide sequence of the corresponding capture probe;

5) for each sample, detecting any binding partner complex bound to the array; and

6) comparing the levels of the binding partner complex detected in each sample to thereby identify one or more ligands that are present at different levels in the samples.

The invention also encompasses methods for generating a ligand profile for one or more of samples. The methods involve identifying ligands that are present at different levels in the samples as described above, and comprise the additional step of generating a ligand profile for one or more of the samples, where the ligand profile comprises values representing the level of one or more ligands that are present at different levels in the samples being compared. In some embodiments, the ligand profile generated for the samples may be used as a reference profile for identifying other populations of test ligands that are of the same type as the samples used to generate the reference profile.

For example, in one embodiment the present invention provides a method of identifying a sample, wherein said method comprises the steps of:

1) providing one or more reference profiles, wherein each reference profile is characteristic of a particular type of sample and comprises values corresponding the levels of ligand of interest in the sample;

2) providing a ligand profile for the test sample, wherein said ligand profile is generated according to one of the methods above and comprises values representing the level of one or more ligands of interest for which values are also comprised within the reference profiles; and

3) determining whether the ligand profile from the test sample is similar to one or more reference profiles to thereby identify the test sample.

In other embodiments, a reference profile comprising values representing the level of one or more ligands that are present at different levels in two or more samples may be generated. Such reference profiles allow different samples to be distinguished by comparing the values comprised in the reference profile with values obtained for the ligands in a population of test ligands. Accordingly, in another embodiment, the present invention provides a method for identifying a test sample, where the method comprises:

1) providing a ligand profile for the test sample, wherein the ligand profile is generated according to the methods described above;

2) providing one or more reference profiles, wherein each reference profile is characteristic of a particular type of sample, and wherein the ligand profile for the test sample and each reference profile comprise one or more values representing the level of a ligand that is present at different levels in the populations of test ligands being compared; and

3) selecting the reference profile that is most similar to the ligand profile for the test sample to thereby identify the test sample.

In some embodiments, a ligand that is present at different levels in two or more populations of test ligands is present at different concentrations in the populations of test ligands. In other embodiments, the ligand is present in one or more populations of test ligands but is absent from other populations of test ligands. When a ligand is absent from a population of test compounds, no binding partner complex will be observed in the population of test compounds. In still other embodiments, a ligand may be present at similar concentrations in the populations of test ligands, but may be modified differently in the populations of test compounds to be compared. Surrogate antibodies that specifically bind to ligands containing a particular modification may be identified using methods described elsewhere herein.

Where the number of different ligands of interest whose levels are measured is large, an algorithm may be used to compare the levels in each population of test ligands to identify patterns of ligands that are present at different levels in the populations of test ligands. Such algorithms are known in the art, and are reviewed, for example, in Slonim (2002) Nature Genetics Suppl. 32:502-508, which is herein incorporated by reference in its entirety.

The methods of identifying one or more ligands that are present at different levels in two or more populations of test ligands may be used to produce a ligand profile that is characteristic of a particular sample. A ligand profile that is characteristic of a particular type of population of test ligands (sample) is termed a “reference profile.” Once the reference profile for a particular reference sample is established, it may be used to determine whether a test sample is of the same sample type as the reference sample. A ligand profile from a test sample is compared to the reference profile to determine whether the test sample ligand profile is sufficiently similar to the reference profile. Alternatively, the test sample ligand profile is compared to a plurality of reference expression profiles to select the reference ligand profile that is most similar to the test sample ligand profile.

The strength of the correlation between the level of ligand that is present at different levels in two or more samples and the identification of a particular type of sample may be determined by a statistical test of significance. Such statistical tests provide a score indicating the strength of the correlation of the level of the ligand and the identification of the type of sample. Such scores may be used to select the ligands whose levels have the greatest correlation with a particular type of sample in order to increase the diagnostic or prognostic accuracy of the ligand profile, or in order to reduce the number of values contained in the ligand profile while maintaining the diagnostic or prognostic accuracy of the ligand profile.

In some embodiments, the reference profile is established using surrogate antibody molecules that bind to known ligands of interest. However, it is recognized that a reference profile that is characteristic or diagnostic of (i.e. capable of identifying) a particular sample type may be developed using ligands whose identity is unknown. Accordingly, in other embodiments, the population of surrogate antibodies is randomized, and the ligands of interest are any ligands that are differentially expressed between the samples undergoing comparison.

Reference profiles may be used to identify a wide variety of samples. For example, reference profiles may be used to identify samples containing an agent of biological or chemical warfare (e.g. Francisella tularensis, Yersinia pestis, Bacillus anthracsis, Ebola virus, Marburg virus, Hanta virus). One advantage of the present invention in such applications is the ability to generate to rapidly generate surrogate antibodies that bind to a particular ligand of interest, allowing the user to rapidly respond to and detect new genetically engineered biowarfare agents. The reference profiles of the invention may also be used to identify environmental samples containing, for example, PCB's, petroleum hydrocarbons, dioxins, to identify food samples, containing, for example aflatoxin, PCBs, dioxins, Salmonella, E. coli 0157, Shigatoxins, Listeria; or to identify samples containing genetically-modified organisms.

It is an advantage of the present invention that the surrogate antibodies are capable of binding to a wide variety of ligands. Accordingly, a reference profile of the invention may comprise values representing the levels of many different types of ligands, including compounds, cells, and viruses.

In a biological sample, differential expression of ligands could result, for example, from differences at any stage of protein expression from transcription through post-translational modification. In addition to being used to quantitate the level of a particular nucleic acid molecule or polypeptide in a biological sample, the surrogate antibodies of the invention may be designed or selected to bind to proteins containing particular post-translational modifications. Examples of such modifications include, but are not limited to, the addition of a phosphate (phosphorylation), carbohydrate (glycosylation), ADP-ribosyl (ADP ribosylation), fatty acid (prenylation, which includes but is not limited to: myristoylation and palmitylation), ubiquitin (ubiquitination) and sentrin (sentrinization; a ubiquitination-like protein modification) or the proteolytic digestion of a protein (proteolysis). Additional examples of protein modifications that may be detected using the surrogate antibodies of the invention include methylation, acetylation, hydroxylation, iodination, and flavin linkage.

The methods may be used to detect molecules that are differentially expressed between two cell types. The two cell types could be normal versus pathologic cells, e.g., cancer cells or cells at different levels or cells at different stages of development or differentiation, or in different parts of the cell cycle. However, the method also is useful in examining two cells of the same type exposed to different conditions. For example, the method is useful in toxicology screening and testing compounds for the ability to modulate gene expression in a cell. In such a method, one biological sample is exposed to the test compound, and other cell is not. Then, the ligand profiles of the samples are compared.

The methods are also useful for identifying diagnostic markers of disease. Proteins that are differentially expressed in a patient sample or a diseased cultured cell compared to normal samples or cells may be diagnostic markers. In general, it is best to compare samples from a statistically significant patient population with normal samples. In this way, information can be pooled to identify diagnostic markers common to all or a significant number of individuals exhibiting the pathology.

A ligand profile may also indicate the presence of a particular pathogen or pathogen strain in the sample, or may be correlated with and used to predict susceptibility to a particular disease or susceptibility to undesirable side effects in response to a given therapy. A “ligand profile” is a collection of values representing the absolute or the relative level of one or more ligands that are present at different levels in two or more samples. Preferably, a ligand profile will contain a sufficient number of values such that the profile can be used to distinguish one sample from another, or to distinguish subjects in one risk group from those in another risk group. In some embodiments, a single value may be sufficient to distinguish one sample of test compounds from another.

C. Methods of Using Arrays in Screening Assays

The compositions and methods of the present invention may be used to screen test compounds to identify target compounds, cells, or viruses that interact with a particular ligand of interest. These screening assays rely the ability of the target compound to prevent or disrupt the formation of a binding partner complex between the ligand of interest and the corresponding surrogate antibody molecule.

In one embodiment, the present invention provides a method for screening test compounds, test cells, or test viruses to identify one or more target compounds, target cells, or target viruses that interact with a ligand of interest, the method comprising:

1) providing an array having a population of capture probes, where the capture probes are attached to discrete, known locations on a solid support, the capture probes comprise a known capture nucleotide sequence, and a population of surrogate antibody molecules are bound to the capture probes by an interaction between the capture nucleotide sequence and a recognition nucleotide sequence comprised within an oligonucleotide tail of the surrogate antibody, where the surrogate antibody molecules further comprise:
a) a specificity strand having a specificity domain flanked by a first constant domain and a second constant domain; and
b) a stabilization strand comprising a first stabilization domain that interacts with said first constant domain and a second stabilization domain that interacts with said second constant domain;

2) contacting the ligand of interest with one or more test compounds, test cells, or test viruses under conditions that allow for the formation of a ligand-test compound complex, a ligand-test cell complex, or a ligand-test virus complex;

3) contacting the ligand of interest of step 2) with the array under conditions that will allow for the formation of a binding partner complex between at least one surrogate antibody molecules bound to the array and the ligand of interest but will not allow for the formation of a binding partner complex between the surrogate antibody molecule and the ligand-test compound complex, the ligand-test cell complex, or the ligand-test virus complex;

4) detecting any binding partner complexes; and

5) comparing the level of binding partner complex formed in the presence and absence of the test compound to thereby determine whether the test compound is a target compound that interacts with the ligand of interest.

In another embodiment, the method for screening test compounds to identify a target compound that binds a ligand of interest comprises

1) contacting one or more ligands of interest with a population of test compounds, test cells, or test viruses under conditions that allow the formation of a ligand-test compound complex, a ligand-test cell complex, or a ligand-test virus complex;

2) contacting a the ligand of interest of step 2) with a population of surrogate antibody molecules under conditions that allow for the formation of a binding partner complex between the ligand of interest and at least one surrogate antibody molecule, but do not allow for the formation of a binding partner complex between a surrogate antibody molecule and the ligand-test compound complex, ligand-test compound complex, or ligand-test virus complex, where the surrogate antibody molecule comprises
a) a specificity strand having a specificity domain flanked by a first constant domain and a second constant domain;
b) a stabilization strand comprising a first stabilization domain that interacts with said first constant domain and a second stabilization domain that interacts with said second constant domain; and,
c) at least one oligonucleotide tail comprising a recognition nucleotide sequence that is unique to the particular surrogate antibody molecule;

3) providing an array comprising a population of capture probes attached to a solid support, where the capture probes are attached to discrete, known locations on the solid support and comprise a capture nucleotide sequence that is complementary to at least one recognition nucleotide sequence;

4) contacting any binding partner complex formed in step 2) with the array under conditions that allow for the hybridization of the recognition nucleotide sequence of the surrogate antibody with the capture nucleotide sequence of the corresponding capture probe;

5) detecting any binding partner complex bound to the array to thereby detect the ligand of interest; and

6) comparing the level of binding partner complex formed in the presence and absence of the test compound, test cell, or test virus, to thereby determine whether the test compound, test cell, or test virus is a target compound, target cell, or target virus that interacts with the ligand of interest.

The present invention also provides a method for screening test compounds to identify a target compound that modulates the level of a ligand of interest. The method comprises the steps of:

1) contacting a first sample containing the ligand of interest with the test compounds;

2) providing a second sample containing the ligand of interest, where the second sample has not been contacted with the test compounds;

3) contacting the first sample and the second sample with a surrogate antibody molecule that is capable of forming a binding partner complex with the molecule of interest, wherein the surrogate antibody molecule comprises
a) a specificity strand having a specificity domain flanked by a first constant domain and a second constant domain;
b) a stabilization strand comprising a first stabilization domain that interacts with said first constant domain and a second stabilization domain that interacts with said second constant domain; and,
c) an oligonucleotide tail comprising a recognition nucleotide sequence that is unique to the particular surrogate antibody molecule;

4) forming a binding partner complex;

5) providing an array comprising a population of capture probes attached to a solid support, where the capture probes are attached to a discrete know region of the solid support and comprise a capture nucleotide sequence that is complementary to at least one recognition nucleotide sequence;

6) contacting the binding partner complex with the array under conditions that allow for the hybridization of the recognition nucleotide sequence of the surrogate antibody with the capture nucleotide sequence of the corresponding capture probe; and

7) detecting the binding partner complex bound to the array; and

8) comparing the levels of the ligand of interest in the first sample and the second sample to thereby determine whether the test compound is a target compound that modulates the levels of the ligand of interest.

The tests compounds used in the methods can be obtained using any of the numerous approaches in combinatorial library methods known in the art, including: biological libraries; spatially addressable parallel solid phase or solution phase libraries; surrogate library methods requiring deconvolution; the ‘one-bead one-compound’ library method; and surrogate library methods using affinity chromatography selection. The biological library approach is limited to polypeptide libraries, while the other four approaches are applicable to polypeptide, non-peptide oligomer or small molecule libraries of compounds. See, for example, Lam (1997) Anticancer Drug Des. 12:145.

Examples of methods for the synthesis of molecular libraries can be found in the art, for example in DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten (1992) Biotechniques 13:412-421), or on beads (Lam (1991) Nature 354:82-84), chips (Fodor (1993) Nature 364:555-556), bacteria (U.S. Pat. No. 5,223,409), spores (U.S. Pat. No. 5,223,409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. U.S.A. 97:6378-6382); (Felici (1991) J. Mol. Biol. 222:301-310).

Candidate compounds include, for example, 1) peptides such as soluble peptides, including Ig-tailed fusion peptides and members of random peptide libraries (see, e.g., Lam et al. (1991) Nature 354:82-84; Houghten et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries; 5) zinc analogs; 6) leukotriene A₄ and derivatives; 7) classical aminopeptidase inhibitors and derivatives of such inhibitors, such as bestatin and arphamenine A and B and derivatives; 8) and artificial peptide substrates and other substrates, such as those disclosed herein above and derivatives thereof.

The methods may be used, for example, to identify candidate drugs that bind to or modulate the levels of a particular drug target. The methods of the invention may also be used to screen candidate drugs to determine whether they interact with molecules other than the known target. Such methods are useful for identifying candidate drugs that are highly selective for the drug target and are less likely to have undesired side effects in drug therapy. Accordingly, the methods of the invention are useful for identifying novel candidate drugs that bind specifically to a particular molecular target, and for determining the molecular selectivity of known or candidate drugs.

Methods of Generating Surrogate Antibody Libraries

The methods of the invention employ populations of surrogate antibody molecules. In some embodiments, the population of surrogate antibodies comprises a library. A library of surrogate antibody molecules is a mixture of stable, pre-formed, surrogate antibody molecules of differing sequences, from which antibody molecules able to bind a desired ligand are captured. As used herein, a library of surrogate antibody molecules comprises a population of molecules comprising a specificity strand and a stabilization strand. The specificity strand comprises a nucleic acid sequence having a specificity region flanked by a first constant region and a second constant region; and, the stabilization strand comprises a first stabilization domain that interacts with said first constant region and a second stabilization domain that interacts with said second constant region. In addition, each of the first constant domains of the specificity strands in the population are identical; each of the second constant domains of the specificity strands in the population are identical; each of the specificity domains of the specificity strands in said population are randomized; and, each of the stabilization strands in said population are identical. It is recognized that a library of surrogate antibody molecules having any of the diverse structures, described elsewhere herein, can be assembled.

As used herein, a library typically includes a population having between at least 2 up to at least 1×10¹⁴ surrogate antibodies. Alternatively, the surrogate antibody library used for selection can include a mixture of between about 2 and about 10¹⁸, between about 10⁹ and about 10¹⁴, between about 10⁹ and about 10²⁴, between about 2 and 10²⁷ or more surrogate antibodies having a contiguous randomized sequence of at least 10 nucleotides in length in each binding cavity (i.e., specificity domain). In yet other embodiments, the library will comprise at least 10, 100, 1000, 10000, 1×10⁵, or 1×10⁶, 1×10⁷, 1×10¹⁰, 1×10¹⁴, 1×10¹⁸, 1×10²², 1×10²⁵, 1×10²⁷ or greater surrogate antibody molecules having a randomized or semi-random specificity domain. The molecules contained in the library can be found together in a mixture, in a collection of single clones or pools of clones (e.g., in the wells of a multiwell plate), or on an array as described elsewhere herein.

The term “population of surrogate antibodies” may be used to refer to polyclonal or monoclonal surrogate antibody preparations of the invention having one or more selected characteristics. A polyclonal surrogate antibody library or “population of polyclonal antibodies” comprises a population of individual clones of surrogate antibodies assembled to produce polyclonal libraries with enhanced binding to a target ligand. Once a surrogate antibody, or a plurality of separate surrogate antibody clones, are found to meet target performance criteria (e.g., bind to a ligand of interest such as a protein of interest) they can be assembled into polyclonal reagents that provide multiple epitope recognition and greater sensitivity and/or avidity in detecting the target ligand. It is recognized that a population of polyclonal surrogate antibodies can represent a pool of molecules obtained following the capture and amplification steps to a desired ligand. Alternatively, a population of polyclonal surrogate antibodies could be formed by mixing at least two individual monoclonal surrogate antibody clones having the desired ligand binding characteristics.

In some embodiments, the binding specificity of one or more members of the population of surrogate antibodies is unknown. Populations of antibodies having unknown binding affinities may be used, for example, to create a ligand profile that is characteristic of a particular type of sample. In other embodiments, one or more of the surrogate antibodies in the population of surrogate antibodies has a known binding affinity. By “known binding specificity”, it is intended that the ligand to which the surrogate antibody binds has been identified. A surrogate antibody molecule that has a known binding specificity for a particular ligand of interest can be used in the methods and compositions of the present invention. Surrogate antibody molecules that bind a ligand of interest can be identified by screening a library of surrogate antibody molecules and capturing surrogate antibody molecules in the population based on their ability to interact with a desired binding partner or ligand.

A library of surrogate antibody molecules comprises a population of molecules comprising a specificity strand and a stabilization strand. The specificity strand comprises a nucleic acid sequence having a specificity region flanked by a first constant region and a second constant region; and, the stabilization strand comprises a first stabilization domain that interacts with said first constant region and a second stabilization domain that interacts with said second constant region. In addition, each of the first constant regions of the specificity strands in the population are identical; each of the second constant region of the specificity strands in the population are identical; each of the specificity region of the specificity strands in said population are randomized; and, each of the stabilization strands in said population are identical. It is recognized that a library of surrogate antibody molecules having any of the diverse structures, described elsewhere herein, can be assembled. In order to identify surrogate antibody molecules that bind to a compound or ligand of interest, the library of surrogate antibodies undergoes a series of iterative in vitro selection steps that allow for the identification and capture of one or more surrogate antibodies that interact with the desired binding partner or ligand. Each round of selection produces a population of surrogate antibody molecules that have an increased binding affinity, increased binding specificity, or both an increased binding affinity and specificity for the compound or ligand of interest as described in more detail below.

A. Forming the Randomized Population of Specificity Regions

Methods of producing a population of specificity strands having randomized specificity domains are known in the art. For example, the specificity domain can be prepared by the synthesis of randomized nucleic acid sequences or by selection from randomly cleaved cellular nucleic acids. Alternatively, full or partial sequence randomization can be readily achieved by direct chemical synthesis of the specificity domain (or portions thereof) or by synthesis of a template from which the specificity domain (or portions thereof) can be prepared by using appropriate enzymes. See, for example, Breaker et al. (1997) Science 261:1411-1418; Jaeger et al. (1997) Methods Enzy 183:281-306; Gold et al. (1995) Annu Rev Biochem 64:763-797; Perspective Biosystems (1998) and Beaucage et al. (2000) Current Protocols in Nucleic Acid Chemistry John Wily & Sons, N.Y. 3.3.1-3.3.20; all of which are herein incorporated in their entirety by reference. Alternatively, the oligonucleotides can be cleaved from natural sources (genomic DNA or cellular RNA preparations) and ligated between constant regions.

The library can include as large a number of specificity domains as is practical for selection, to insure that a maximum number of potential binding sequences are identified. For example, if the randomized sequence in the specificity domains includes 30 nucleotides, it would contain approximately 10¹⁸ (i.e. 4³⁰) sequence permutations using the 4 naturally occurring deoxyribonucleotides, and an even greater number of sequence permutations if modified nucleotides are included.

In some embodiments, a bias can be deliberately introduced into a randomized sequence, for example, by altering the molar ratios of precursor nucleoside (or deoxynucleoside) triphosphates of the synthesis reaction. A deliberate bias may be desired, for example, to approximate the proportions of individual bases in a given organism, or to affect secondary structure. See, Hermes et al. (1998) Gene 84:143-151 and Bartel et al. (1991) Cell 67:529-536, both of which are herein incorporated by reference. See also, Davis et al. (2002) Proc. Natl. Acad. Sci. 99:11616-11621, which generated a randomized population having a bias comprising a specified stem loop structure. Thus, as used herein, a randomized population of specificity domains may be generated to contain a desirable bias in the primary sequence and/or secondary structure of the domain. In other embodiments, the length of the specificity region of individual members within the library can be substantially the same or different. Iterative libraries can be used, where the specificity domain varies in size in each library or are combined to form a library of mixed loop sizes, for the purpose of identifying the optimum loop size for a particular target ligand.

As discussed above, the specificity strand may contain various functional moieties and modifications. Methods of forming the randomized population of specificity strands will vary depending on the functional moieties that are to be contained on the strand. For example, in one embodiment, the functional moieties comprise modified adenosine residue. In this instance, the specificity strand could be designed to contain adenosine residues only in the specificity domain. The nucleotide mixture used upon amplification will contain the adenosine having the desired functional moieties (i.e., moieties that increase hydrophobic binding characteristics). In other instances, the functional moiety can be attached to the surrogate antibody following the synthesis reaction.

B. Generating a Surrogate Antibody Library

Generating a library of surrogate antibody molecule comprises: a) providing a population of specificity strands where i) the specificity strands comprise a specificity domain flanked by a first constant domain and a second constant domain; ii) the first constant domains of the specificity strands in the population are identical; iv) the second constant domains of the specificity strands in said population are identical. The population of specificity strands is contacted with a stabilization strand; wherein the stabilization strand comprises a first stabilization domain that interacts with the first constant domain of the specificity strand and a second stabilization domain that interacts with the second constant domain of the specificity strand. The population of specificity strands is contacted with the stabilization strand under conditions that allow for the first stabilization domain to interact with the first constant domain and the second stabilization domain to interact with the second constant domain. In some embodiments the specificity strand and stabilization strand are comprises within the same, contiguous nucleic acid strand, while in other embodiments the specificity strand and stabilization strand are comprised within non-contiguous nucleic acid strands.

In some embodiments, it may be preferable to produce a population of surrogate antibodies having a randomized specificity domain that varies in length. This allows the library to be used in a “multi-fit” process of surrogate antibody development that defines the optimal surrogate ligand binding cavity size to use for any given target. The process allows surrogate antibody binding to improve upon the binding characteristics of native antibody molecules where the size of the paratope (binding site) is finite for all ligands regardless of size. The “multi-fit” process identifies a cavity size with spatial characteristics that enhance the fit, specificity, and affinity of the surrogate antibody-ligand complex. The “multi-fit” process can identify as an ideal binding loop/cavity one that is not restricted in size or dimensionality by the precepts of evolution and genetics. As such, surrogate antibody molecules challenge the conventional paradigm regarding the size of an epitope or determinant as shaped by the dependency of science and research on the properties of native antibody molecules. Preliminary “multi-fit” ligand capture rounds are performed using a heterogeneous population of surrogate antibodies containing specificity domains of varying size and conformation. The optimal cavity size for surrogate library preparation is indicated by the sub-population having a cavity size that exhibits the highest degree of ligand binding after a limited number of capture and amplification cycles.

C. Methods of Screening a Surrogate Antibody Library

Methods that allow the surrogate antibody library or a selected population of surrogate antibodies to be screened to identify or “capture” a surrogate antibody or a population of surrogate antibodies having the desired ligand-binding characteristics are provided. In this manner, surrogate antibody molecules are selected for subsequent cloning from a library of pre-synthesized multi-stranded molecules that contain a random specificity region and stabilization regions that stabilize the structure of the molecule in solution.

Generally, surrogate antibodies that bind to a particular target/ligand are captured from a starting surrogate antibody library by contacting one or more ligand with the library, binding one or more surrogate antibodies to the ligand(s), separating the surrogate antibody bound ligand from unbound surrogate antibody, and identifying the bound ligand and/or the bound surrogate antibodies.

For example, in one embodiment, a library of surrogate antibody molecules can be screened by

1) contacting at least one ligand of interest with the library of surrogate antibody molecules to allow a binding partner complex to form between at least one of the surrogate antibody molecules and the ligand of interest;

2) partitioning the unbound ligand and the unbound members of the population of surrogate antibody molecules from said population of ligand-bound surrogate antibody complexes; and

3) amplifying the specificity strand of the population of ligand-bound surrogate antibody complexes to thereby identify a surrogate antibody molecule that binds to the ligand of interest.

The methods allow for the selection or capturing of a surrogate antibody molecule that interacts with the desired ligand of interest. The method thereby employs selection from a library of surrogate antibody molecules followed by step-wise repetition of selection and amplification to allow for the identification of the surrogate antibody molecule have the desired binding affinity and/or selectivity for the ligand of interest. As used herein a “selected population of surrogate antibody molecules” is intended a population of molecules that have undergone at least one round of ligand binding and partitioning.

In another embodiment, the method of capturing a surrogate antibody comprises contacting a selected population of surrogate antibodies with the ligand of interest. In this embodiment, the surrogate antibody antibodies comprise specificity domains that have been selected for increased affinity, increased specificity, or both increased affinity and increased specificity for the ligand of interest by at least two, at least three, at least four, at least five, at least six, at least seven, at least eight, at least nine, or at least ten or more rounds of selection and amplification. The surrogate antibody molecules that bind to the ligand of interest may then be captured by

1) contacting a ligand of interest with a population of surrogate antibody molecules under conditions that permit formation of a binding partner complex between the ligand of interest and one or more surrogate antibody molecules;

2) partitioning the unbound ligand of interest and the unbound members of the population of surrogate antibody molecules from the binding partner complex; and

3) amplifying the specificity strand of the surrogate antibody molecule comprised in the binding partner complex.

In some embodiments, a population of selected surrogate antibody molecules is produced from the amplified specificity strand by contacting the amplified specificity strand with a stabilization strand under conditions that allow for the first stabilization domain of the stabilization strand to interact with said first constant domain of the specificity strand and said second stabilization domain of the stabilization strand to interact with the second constant domain of the specificity strand.

D. Methods of Contacting a Surrogate Antibody Molecule with a Ligand of Interest to Form a Binding Partner Complex

In some embodiments of the methods of the present invention, a surrogate antibody molecule is contacted with a ligand or compound under conditions that allow for the formation of a binding partner complex between the surrogate antibody molecule and the ligand or compound. One of skill in the art will recognize that a variety of conditions could be used to allow formation of the binding partner complex. In some embodiments the surrogate antibody molecule that binds to the ligand of interest is selected under conditions similar to those found in the environment in which the ligand of interest would be found in vivo or the anticipated in vitro application. Conditions that can be adjusted to accurately reflect this in vivo or in vitro binding environment include, but are not limited to, temperature, total ionic strength (osmolarity), pH, enzyme composition (e.g. the presence of nucleases), the presence of metalloproteins (e.g. hemoglobin, ceruloplasm), and the presence of irrelevant compounds. See, for example, Dang et al. (1996) J Mol Bio 264:268-278; O'Connell et al. (1996) Proc. Natl. Acad Sci USA 93:5883-7; Bridonneu et al. (1999) Antisense Nucleic Acid Drug Dev 9:1-11; Hicke et al. (1996) J Clin Investig 98:2688-92; and, Lin et al. (1997) J Mol Biol 271:446-8, all of which are herein incorporated by reference. For example, when selecting a surrogate antibody to be used in the methods of the present invention, it may be desirable to select under conditions conducive to the hybridization of the recognition nucleotide sequence of the surrogate antibody with the capture nucleotide sequence of the capture probe.

The ligand of interest may be any ligand that interacts with a surrogate molecule of the invention. Examples of ligands of interest include, but are not limited to, immunological haptens, environmental pollutants and toxins (e.g., polychlorinated biphenyls, dioxins, polyaromatic hydrocarbons), explosives, allergens, poisons, natural or surrogate polymers, carbohydrates, polysaccharides, muccopolysaccharides, glycoproteins, enzymes, antigens, molecules (e.g. proteins, nucleic acid molecules, carbohydrates, or metabolites) derived from a bacteria, biomolecules (e.g. proteins, nucleic acid molecules, or carbohydrates) derived from a virus, therapeutic agents, illicit drugs and substances of abuse (e.g., narcotics) hormones (e.g., thyroxin), peptides, polypeptides, prions, and nucleic acids. A ligand can also be a cell or its constituents, for example, a pathogen or one or more cellular organelles. Additional ligands of interest include molecules whose levels are altered in tumors (i.e., growth factor receptors, cell cycle regulators, angiogenic factors, and signaling factors). Accordingly, the surrogate antibody molecules of the invention can be produced for the detection of any ligand of interest.

Appropriate conditions for contacting the ligand of interest and the surrogate antibody can be determined empirically based on the reaction chemistry. In general, the appropriate conditions will be sufficient to allow 1% to 5%, 5% to 10%, 10% to 20%, 20% to 40%, 40% to 60%, 60% to 80%, 80% to 90%, or 90% to 100% of the antibody molecule population to interact with the ligand.

E. Methods of Partitioning the Binding Partner Complex from Unbound Ligand and Unbound Surrogate Antibody Molecules.

By “partitioning” is intended any process whereby surrogate antibody bound to target ligands (ligands of interest), termed ligand-bound surrogate antibody complexes or binding partner complexes, are separated from surrogate antibodies that are not bound to target, or from unbound ligands. Partitioning allows for the separation of the surrogate antibodies into at least two pools based on their relative affinity to the target ligand. Methods for partitioning are known in the art. For example, surrogate antibodies bound to ligands of interest can be immobilized onto a surface, or may be filtered through molecular sieves that retain the binding partner complex but not the unbound surrogate antibody molecules or unbound ligand. Columns that specifically retain ligand-bound surrogate antibody can be used for partitioning. Liquid-liquid partition can also be used as well as filtration gel retardation, and density gradient centrifugation. The choice of the partitioning method will depend on properties of the target/ligand and on the ligand-bound surrogate antibody and can be made according to principles and properties known to those of ordinary skill in the art.

In one embodiment, partitioning comprises filtering a mixture comprising the unbound ligand, the population of unbound surrogate antibody molecules, and the population of ligand-bound surrogate antibody complexes through a filtering system wherein said filtering system retains the ligand-bound surrogate antibody complex in the retinate and allows the unbound surrogate antibodies to pass into the filtrate. Such filtering systems are known in the art. For example, filtration membranes that separate on the basis of size (e.g. Amicon Microcon®D, Pall Nanosep®), charge, hydrophobicity, chelation, or clathration may be used.

The pore size used in size-exclusion filtration will be determined by the size of the ligand of interest and the size of the surrogate antibody molecules population of surrogate antibodies. For example, a cellular ligand having a 7-10 micron diameter will be retained by a membrane that excludes 7 microns. When such a membrane is used, surrogate antibody molecules having a 120 nucleotide bi-oligonucleotide structure when uncomplexed are easily eliminated as they pass through the membrane. Those bound to the ligand are captured in the retentate and used for assembly of the subsequent selected population. The preparation of a surrogate antibody to a BSA-hapten conjugate must use a pore that excludes the surrogate antibody-conjugate complex. A membrane that excludes 50,000 or 100,000 daltons effectively fractionates this surrogate antibody when bound to the conjugate from free surrogate antibody. Surrogate antibody prepared to a small protein, such as the enzyme Horseradish Peroxidase requires a membrane that would exclude molecules that are approximately 50,000 daltons or greater, while allowing the uncomplexed surrogate antibody to penetrate the filter. Target ligands can be chemically conjugated to larger carrier molecules or polymerized to enhance their size and membrane exclusion characteristics.

Alternative protocols that may be used to separate surrogate antibodies bound to target ligands from unbound surrogate antibodies and unbound ligand are known in the art. For example, the separation of ligand-bound and free surrogate antibody molecules that exist in solution can be achieved using size exclusion column chromatography, reverse phase chromatography, size exclusion/molecular sieving filtration, affinity chromatography, solid phase chromatography (C18, hydroxyapatite, chelation, adsorbed metals), electrophoretic methods, ion exchange chromatography, solubility modification (e.g. ammonium sulfate or methanol precipitation), immunoprecipitation, protein denaturation, fluorescence activated cell sorting (FACS), density gradient centrifugation. Ligand-bound and unbound surrogate antibody molecules can be separated using analytical methods such as HPLC and fluorescent activated cell sorters.

Affinity chromatography procedures using selective immobilization to a solid phase can be used to separate surrogate antibody bound to a target ligand from unbound surrogate antibody molecules. Such methods could include immobilization of the target ligand onto absorbents composed of agarose, dextran, polyacrylamide, glass, nylon, cellulose acetate, polypropylene, polyethylene, polystyrene, or silicone chips.

Method of amplifying the specificity strand of the surrogate antibody are described below, however, it is recognized that a surrogate antibody bound to the target ligand could be used in PCR amplification to produce one or more oligonucleotide strands having an integral specificity region with or without separation from the affinity matrix.

A combination of solution and solid-phase separation could include binding a surrogate antibody to ligand conjugated microspheres that could be isolated based upon a physicochemical effect created by the surrogate antibody binding. Separate microsphere populations could individually be labeled with chromophores, fluorophores, magnetite conjugated to different target ligands or difference orientations of the same ligand. Surrogate antibody molecules bound to each microsphere population could be isolated on the basis of microsphere reporter molecule characteristic(s), allowing for production of multiple surrogate populations to different ligands simultaneously.

F. Methods of Amplifying the Specificity Strand

Methods for amplifying the specificity strand of a surrogate antibody molecule are provided. By “amplification” is intended one or more steps that increases the amount or number of copies of a molecule or class of molecules. RNA molecules can be amplified by a sequence of three reactions: making cDNA copies of selected RNAs, using polymerase chain reaction to increase the copy number of each cDNA, and transcribing the cDNA copies to obtain RNA molecules having the same sequences as the selected RNAs. Any reaction or combination of reactions known in the art can be used as appropriate, including direct DNA replication, direct RNA amplification and the like, as will be recognized by those skilled in the art. The amplification method should result in the proportions of the amplified mixture being essentially representative of the proportions of different constituent sequences in the initial mixture. While the constant regions on either side of the specificity region in the surrogate antibody molecule stabilize the structure of the specificity region, these regions can also be used to facilitate the amplification of the surrogate antibodies.

In this manner, a population of specificity strands is generated. Thus, when the amplified specificity strands are contacted with the appropriate stabilization stand, a population of surrogate antibodies having the desired ligand binding affinity and/or specificity can be formed. Methods to selectively enhance the specificity of the ligand interaction and methods for enhancing the binding affinity of the population are provided below.

Once a desired surrogate antibody or set of surrogate antibodies is identified, it is often desirable to identify one or more of the monoclonal surrogate antibody clones and generate large amount of either a monoclonal or assembled polyclonal surrogate antibody reagent. Capturing a monoclonal surrogate comprises cloning at least one specificity strand from the population of amplified specificity strands. The cloned specificity strand can be amplified using routine methods and subsequently contacted with the appropriate stabilization strand under conditions that allow for said first stabilization domain to interact with said first constant region and said second stabilization domain to interact with said second constant region, and thereby producing a population of monoclonal surrogate antibodies.

Methods of amplifying nucleic acid sequences (such as those of the specificity strand) are known. The polymerase chain reaction (PCR) is an exemplary method for amplifying nucleic acids. PCR methods are described, for example in Saiki et al. (1985) Science 230:1350-1354; Saiki et al. (1986) Nature 324:163-166; Scharf et al. (1986) Science 233:1076-1078; Innis et al. (1988) Proc. Natl. Acad. Sci. 85:9436-9440; and in U.S. Pat. No. 4,683,195 and U.S. Pat. No. 4,683,202, the contents of each of which are incorporated herein in their entirety.

PCR amplification involves repeated cycles of replication of a desired single-stranded DNA (or cDNA copy of an RNA) employing specific oligonucleotide primers complementary to the 3′ and 5′ ends of the single-stranded DNA, primer extension with a DNA polymerase, and DNA denaturation. Products generated by extension from one primer serve as templates for extension from the other primer. A related amplification method described in PCT published application WO 89/01050 requires the presence or introduction of a promoter sequence upstream of the sequence to be amplified, to give a double-stranded intermediate. Multiple RNA copies of the double-stranded promoter containing intermediate are then produced using RNA polymerase. The resultant RNA copies are treated with reverse transcriptase to produce additional double-stranded promoter containing intermediates that can then be subject to another round of amplification with RNA polymerase. Alternative methods of amplification include among others cloning of selected DNAs or cDNA copies of selected RNAs into an appropriate vector and introduction of that vector into a host organism where the vector and the cloned DNAs are replicated and thus amplified (Guatelli et al. (1990) Proc. Natl. Acad. Sci. 87:1874). In general, any means that will allow faithful, efficient amplification of selected nucleic acid sequences can be used. It is only necessary that the proportionate representation of sequences after amplification at least roughly reflects the relative proportions of sequences in the mixture before amplification. See, also, Crameri et al. (1993) Nucleic Acid Research 21: 4110, herein incorporated by reference. The method can optionally include appropriate nucleic acid purification steps.

In some embodiments, the stabilization strand of the surrogate antibody molecule is modified such that it is not efficiently amplified. Such modifications allow for the preferential amplification of the specificity strand of the antibody.

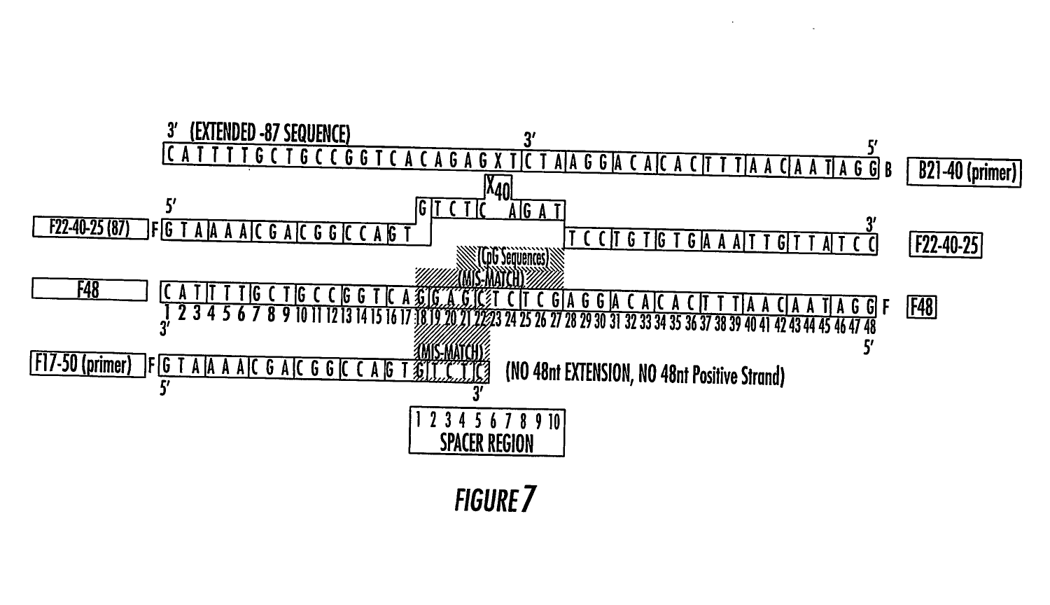

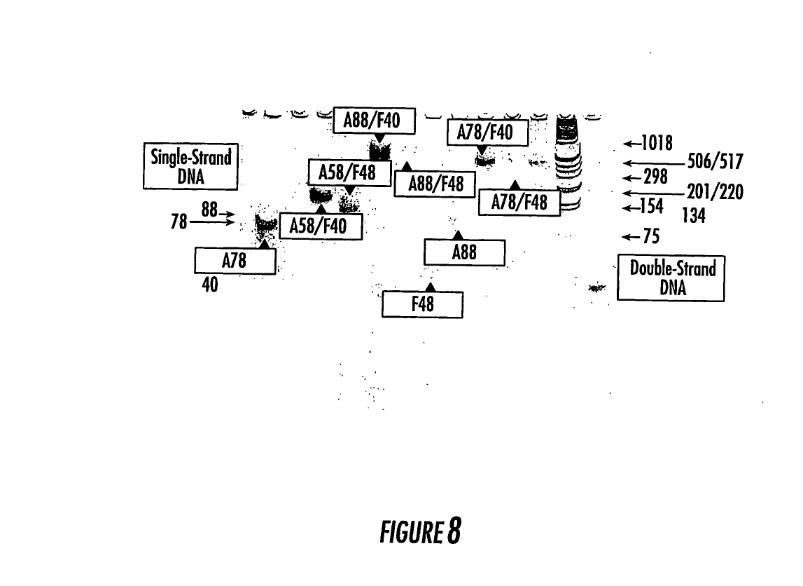

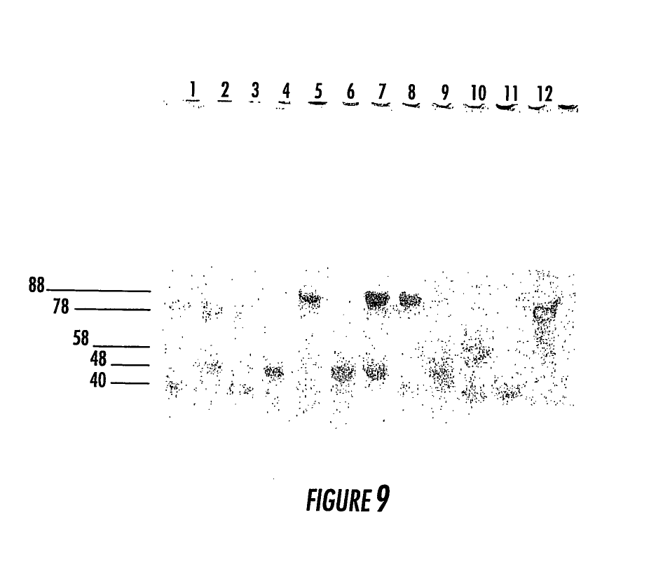

In other embodiments, the stabilization strand and the specificity strand contain a region of non-homology that can be used, in combination with the appropriate primers, to prevent the amplification of the stabilization strand. A non-limiting example of this embodiment appears in FIG. 7 and in Example 4 of the Experimental section. Briefly, in this non-limiting example, the stabilization strand and specificity strand lack homology in about 2, 3, 4, 5, 6, 8 or more nucleotides positioned 5′ to the specificity domain. See, shaded box in FIG. 7. The primer used to amplify the positive strand of the specificity strand is complementary to the sequences of the specificity strand. However, due to the mis-match design, this primer lacks homology at its 3′ end to the sequence of the stabilization strand. This lack of homology prevents amplification of the full-length negative stabilization strand. This method therefore allows for the preferential amplification of the specificity strand.

When the surrogate antibody comprises a stabilization strand and a specificity strand comprising a nucleic acid sequence, each of the strands (i.e., the juxtaposed surrogate antibody strands) that contain a linear array of stabilization sequence(s), constant regions, specificity sequence(s) and/or spacer sequence(s) is initially prepared by a DNA synthesizer. In one embodiment, the selection process for capturing and amplifying a specific, high affinity, surrogate antibody reagent preferentially amplifies only the strand(s) containing specificity region(s) sequence by PCR. As outlined above in more detail, the surrogate molecules are assembled by mixing these strands with the appropriate stabilization strands strand(s) that ensure proper alignment upon interaction of the constant and stabilization domains. Once the juxtaposed strands are mixed the solution is heated and the strands allowed to hybridize as the temperature is reduced. In other embodiments, the surrogate antibody may be formed without heating.

G. Staging of Selected Surrogate Antibody Molecules.

Surrogate antibody molecules that bind to a ligand of interest may be selected by a process of iterative selection for surrogate antibody elements that specifically bind to the selected target molecule with high affinity. This process for the capture and amplification of surrogate antibody molecules that bind a target ligand is referred to herein as “staging.” The staging process can be modified in various ways to allow for the identification of surrogate antibody having the desired affinity and specificity.

For instance, steps can be taken to allow for “specificity enhancement” and thereby eliminate or reduce the number of irrelevant or undesirable surrogate antibody molecules from the captured population. In addition, “affinity enhancement” can be performed and thereby allow for the selection of high affinity surrogate antibody molecules to the target ligand. The staging process is particularly useful in the rapid isolation and amplification of surrogate antibodies that have high affinity and specificity for the target molecule/ligand. See, for example, Crameri et al. (1993) Nucleic Acid Research 21:4410.

Specific binding is a term that is defined on a case-by-case basis. In the context of a given interaction between a given surrogate antibody molecule and a given target, enhanced binding specificity results when the preferential binding interaction of a surrogate antibody with the target is greater than the interaction observed between the surrogate antibody and irrelevant and/or undesirable targets. The surrogate antibody molecules described herein can be selected to be as specific as required using the “staging” process to capture, isolate, and amplify specific molecules.

Accordingly, the present invention further provides a method of enhancing the binding specificity of a surrogate antibody comprising:

a) contacting a population of surrogate antibody molecules, said population of surrogate antibody molecules capable of binding a ligand of interest, with a non-specific moiety under conditions that permit formation of a population of non-specific moiety-bound surrogate antibody complexes,

wherein said surrogate antibody molecule of the surrogate antibody population comprises a specificity strand and a stabilization strand, said specificity strand comprising a nucleic acid sequence having a specificity region flanked by a first constant region and a second constant region; and, said stabilization strand comprises a first stabilization domain that interacts with said first constant region and a second stabilization domain that interacts with said second constant region;

b) partitioning said non-specific moiety and said population of non-specific moiety-bound surrogate antibody complexes from said population of surrogate unbound antibody molecules; and,

c) amplifying the specificity strand of said population of unbound surrogate antibody molecules.

In further embodiments, the method of enhancing the binding affinity further comprises contacting the population of specificity strands of step (c) above with a stabilization strand under conditions that allow for said first stabilization domain to interact with said first constant region and said second stabilization domain to interact with said second constant region.

In further embodiments, the population of surrogate antibodies comprises a library of surrogate antibodies and/or a population of selected antibodies.

In this embodiment, the binding specificity of the surrogate antibody population is enhanced by contacting the population of surrogate antibodies with a non-specific moiety under conditions that permit formation of a population of non-specific moiety-bound surrogate antibody complexes. In this manner, surrogate antibodies that interact with both the target ligand and a variety of non-specific moieties can partitioned from the population of surrogate antibodies having a higher level of specificity to the desired ligand.

By “non-specific moiety” is intended any molecule, cell, organism, virus, chemical compound, nucleotide, or polypeptide that is not the desired target ligand. Depending on the desired surrogate antibody population being produced, one of skill in the art will recognize the most appropriate non-specific moiety to be used. For example, if the desired target is protein X which has 95% sequence identity to protein Y, the binding specificity of the surrogate antibody population to protein X could be enhanced by using protein Y as a non-specific moiety. In this way, a surrogate antibody population with enhanced interaction to protein X could be produced. See, for example, Giver et al. (1993) Nucleic Acid Research 23: 5509-5516 and Jellinek et al. (1993) Proc. Natl. Acad. Sci 90:11227-11231.

Binding affinity is a term that describes the strength of the binding interaction between the surrogate antibody and a ligand. An enhancement in binding affinity results in the increased binding interaction between the target ligand and the surrogate antibody. The binding affinity of the surrogate antibody and target ligand interaction directly correlates to the sensitivity of detection that the surrogate antibody will be able to achieve. In order to assess the binding affinity under practical applications, the conditions of the binding reactions must be comparable to the conditions of the intended use. For the most accurate comparisons, measurements will be made that reflect the interaction between the surrogate antibody and target ligand in solutions and under conditions of their intended application.

Accordingly, the present invention provides method of enhancing the binding affinity of a surrogate antibody comprising:

a) contacting a ligand with a population of surrogate antibody molecules under stringent conditions that permit formation of a population of ligand-bound surrogate antibody complexes,

wherein said surrogate antibody molecule of the surrogate antibody population comprises a specificity strand and a stabilization strand,

said specificity strand comprising a nucleic acid sequence having a specificity region flanked by a first constant region and a second constant region; and,

said stabilization strand comprises a first stabilization domain that interacts with said first constant region and a second stabilization domain that interacts with said second constant region;

b) partitioning said ligand, said population of surrogate antibody molecules from said population of ligand-bound surrogate antibody complexes; and,

c) amplifying the specificity strand of said population of ligand-bound surrogate antibody complexes.

In a further embodiment, the method of enhancing binding affinity further comprises contacting said population of specificity strands of step (c) above with a stabilization strand under conditions that allow for said first stabilization domain to interact with said first constant region and said second stabilization domain to interact with said second constant region.

In further embodiments, the population of surrogate antibodies comprises a library of surrogate antibodies and/or a population of selected surrogate antibodies.

In this embodiment, contacting the desired ligand with a population of surrogate antibody molecules under stringent conditions that permit formation of a population of ligand-bound surrogate antibody complexes, allows for the selection of surrogate antibodies that have increased binding affinity to the desired ligand. By “stringent conditions” is intended any condition that will stress the interaction of the desired ligand with the surrogate antibodies in the population. Such conditions will vary depending on the ligand of interest and the preferred conditions under which the surrogate antibody and ligand will interact. It is recognized that the stringent condition selected will continue to allow for the formation of the surrogate antibody structure. Examples of such stringent conditions include changes in osmolarity, pH, solvent (organic or inorganic), temperature surfactants, or any combination thereof. Additional components could produce stringent conditions include components that compromise hydrophobic, hydrogen bonding, electrostatic, and Van der Waals interactions. For example, 10% methanol or ethanol compromise hydrophobic boning and are water-soluble.

The stringency of conditions can also be manipulated by the surrogate antibody to ligand ratio. This increase can occur by an increase in surrogate antibody or by a decrease in target ligand. See, for example Irvine et al. (1991) J Mol Biol 222:739-761. Additional alterations to increase the stringency of binding conditions include, alterations in salt concentration, binding equilibrium time, dilution of binding buffer and amount and composition of wash. The stringency of conditions will be sufficient to decrease the % antibody bound by 1% to 10%, 10% to 20%, 20% to 30%, 30% to 40%, 40% to 50%, 60% to 70%, 70% to 80%, 80% to 90%, 90% to 99% of the total population.

In yet other embodiments, following the identification and isolation of a monoclonal surrogate antibody that has desirable ligand binding specificity, one of skill could further enhance the affinity of the molecule for the desired purpose by mutagenesizing the specificity region and screening for the tighter binding mutants. See, for example, Colas et al (2000) Proc. Natl. Aca. Science 97:13720-13725. In yet other embodiments, following the identification and isolation of a monoclonal surrogate antibody that has desirable ligand binding specificity, one of skill could further enhance the affinity of the molecule for the desired purpose by mutagenizing the specificity region and screening for the mutants that have the highest affinity for the ligand of interest. See, for example, Colas et al. (2000) Proc. Natl. Acad. Science USA 97:13720-13725.

### EXPERIMENTAL

### Example 1

### Process for Making a Ligand-Binding Surrogate Antibody Reagent

An initial library of “Surrogate Antibody” (SAb) molecules was assembled by hybridizing two oligonucleotide strands of pre-defined sequence that were obtained commercially (Life Technologies). Two microliters (100 pmole/microliter) of a 78 nt oligonucleotide strand having the sequence of “(5′) GTA-AAA-CGA-CGG-CCA-GT-Random 40 nt-TCC-TGT-GTG-AAA-TTG-TTA-TCC (3′)” (SEQ ID NO:5) and two microliters (100 pmole/microliter) of a 40 nt oligonucleotide strand having the sequence of “(5′) Biotin-GGT-TAA-CAA-TTT-CAC-ACA-GGA-GGA-CTG-GCC-GTC-GTTTTA-C (3′)” (SEQ ID NO:6) were mixed in a modified Tris buffer, pH 8.0 containing MgS0₄. The solution was heated to 96° C. using a thermal cycler and allowed to hybridize as the solution was cooled to room temperature. SEQ ID NO:5 comprises the specificity strand. The first constant region is underlined and the second constant region has a double underline. SEQ ID NO:6 represents a stabilization region strand. The first stabilization domain is denoted with a single underline. The second stabilization domain is denoted with a double underline.

A library of 1.2×10¹⁴ surrogate antibody molecules was added to 20 μl (1 μg/μl) of a Bovine Serum Albumin (BSA) Polychlorinated Biphenyl (PCB) conjugate suspended in modified Tris buffer, pH 8.0, containing 10% methanol. The solution was incubated for RT/25° C. and transferred to a MICROCON®-PCR filtration device (Millipore). This filtration device was previously determined to retain SAb molecules bound to the BSA-PCB conjugate and not retain unbound SAb molecules. SAb bound to the conjugate was separated from unbound molecules by centrifuging the incubation solution at 1000 g/10′/RT. The BSA-PCB bound SAb in the retentate was washed three times with 200 μl aliquots of the modified Tris buffer.

SAb in the washed retentate was aspirated (˜40 μl) from the filter and transferred into a PCR Eppendorf tube. The recovered SAb-BSA-PCB complex was used to amplify the 78 nt strand without prior dissociation from the conjugate. DNA polymerase, nucleotide triphosphates (NTP), buffer, and an M13R48 primer specific for the starting positive strand and having the sequence (5′) Biotin-GGA-TAA-CAA-TTT-CAC-ACA-GGA (3′) (SEQ ID NO:7) was used in the polymerase chain reaction (PCR) to first produce an amplified population of 78 nt negative strands (i.e., specificity strand). A thermal cycler was programmed to perform 40 cycles of amplification at temperatures of 96° C., 48° C., and 72° C. for 30-300″.

An amplified population of the positive 78 nt strand was next produced from the amplified 78 nt negative strand material using asymmetric PCR. Approximately 5% of the amplified 78 nt negative strand was added to an Eppendorf PCR tube with 40 μl of DI H₂O. Polymerase, NTP, buffer, and an M13-20 primer specific for the negative strand and having the sequence (5′) Biotin-GTA-AAA-CGA-CGG-CCA-GT (3′) (SEQ ID NO:8) was added and used for PCR amplification. The temperature cycles previously cited were again used. Less than 4% of the amplified population was found to contain either 78 nt negative or 40 nt positive strands. Purification to remove polymerase, NTP, primer and 40 nt oligomers was performed using a commercial product (Qiagen PCR Purification Kit).

Re-assembly of the 120 nt, double-stranded, SAb was performed by hybridizing the captured, amplified, and purified 78 nt strand (i.e., specificity strand) with the 40 nt starting oligonucleotide (i.e., stabilization strand). This reassembly process produces an enriched library of ligand-binding SAb molecules. Enriched SAb libraries are assembled prior to beginning each of the subsequent rounds of selection. These subsequent cycles use a positive selection process to enhance the average specificity and affinity of the SAb population for the target ligand.

Approximately 80% (40 μl) of the purified 78 nt material was added to a 200 μl Eppendorf tube containing modified Tris buffer and 5 μl (10 pmole/ul) of the 40 nt strand. Deionized water (35 μl) was added and the mixture heated to 96° C./5′, 65° C./5′, 60° C./5′, and 56° C./5′. The solution was then allowed to cool at the rate of 1° C./min. for 30′ until it reached RT. The solution was filtered through a Microcon® filtration device (5′/1000 g/RT) and the filtrate was collected for use in a subsequent cycle of selection.

Several capture and amplification selection cycles (i.e. 2-6), each preceded by the amplification of the 78 nt oligonucleotide strand, purification, and SAb assembly, were used to produce an enriched library of BSA-PCB-binding SAb molecules. After completing the capture and amplification cycles, the enriched SAb library was processed to capture and amplify SAb molecules that are specific for the target ligand.

Cycles of specificity selections are used to eliminate SAb molecules in the population that bind carrier proteins, derivative chemistries, or cross-reacting compounds. It results in the production of an enriched SAb population of molecules that specifically bind the target ligand. When producing a SAb population that can specifically bind unique determinants on neoplastic tissue, specificity selections eliminate SAb molecules that bind to normal cell constituents.

The process of separating bound from unbound SAb using the MICROCON® filtration device was used as previously explained. The enriched SAb library produced during the capture and amplification phase was incubated with a solution of unconjugated Bovine Serum Albumin (20 μg/ml) for 60′/RT. The solution was then filtered through a MICROCON® filtration device (5′/1000 g/RT). The filter retains SAb bound to BSA. SAb in the filtrate was recovered and used to amplify the 78 nt strand and assemble and purify a new SAb library. SAb was incubated with solutions containing untargeted PCB congeners (e.g. BZ54, BZ18, etc.), dioxins, polyaromatic hydrocarbons (e.g. naphthalene, phenanthrene) and other irrelevant haptens prior to incubation with the target PCB (BZ101)-BSA conjugate. The incubated solutions containing the SAb, irrelevant ligand(s), and target conjugate are filtered through the MICROCON® filtration device. Non-specific SAb molecules bound to the cross-reacting ligands in solution are not excluded by the porosity of the filter and pass into the filtrate and are discarded. Molecules bound to the PCB-BSA conjugate, after exposure to potential cross-reacting compounds, are retained by the membrane and are processed into a new SAb population. These molecules are used to amplify the 78 nt strand and assemble a specific population of SAb molecules that are then used in cycles of sensitivity selections to capture the highest binding affinity molecules.

Cycles of sensitivity selections are used to capture the highest affinity SAb molecules from a library of specific binding molecules for the purpose of preparing a specific, high affinity, polyclonal SAb library. The process exposes the SAb library produced after cycles of specificity selections to reduced concentrations of the target ligand and agents and conditions that compromise hydrophobic, electrostatic, hydrogen, Van der Waals binding interactions. Such agents and conditions include solvents (e.g. methanol), pH modifications, chaotropic agents (e.g. guanidine hydrochloride), elevated salt concentrations, surfactants (e.g. tween, triton) that can be used alone or in combination. The process compromises ligand binding and selects for the highest binding affinity molecules. Once selected these molecules are used as a template to amplify the 78 nt strand and assemble an enriched polyclonal population.

Sensitivity selections are performed using the enriched SAb population obtained after completing the “capture and amplification” and “specificity selections”. The solution-phase process of capturing, or eliminating, SAb on the basis of their binding to a ligand and capture using a molecular sieving filtration device was again used. The SAb was incubated with unconjugated PCB molecules prior to the addition of the BSA-PCB (BZ101) conjugate for 60′/RT. The incubation solution was introduced into a MICROCON® filtration device and centrifuged at 1000 g/10′/RT. SAb bound to the unconjugated PCB molecules proceed into the filtrate where they are collected and used to amplify the 78 nt strand and assemble an enriched population of molecules that bind the unconjugated ligand. The enriched population was incubated with the PCB-BSA conjugate at a reduced concentration (0.4 μg/ml) and SAb bound to the conjugate are recovered after filtration using the MICROCON® device (1000 g/10′/RT) and washing three times using a modified Tris buffer containing 0.05% Tween 20. Recovered SAb in the retentate was amplified to produce 78 nt strands and assembled into SAb molecules. The process was repeated by incubating the SAb library with the PCB-BSA (0.4%) conjugate in the presence of methanol (10% v/v) and Tween 20 (0.05%). SAb bound to the conjugate was recovered in the retentate and used to amplify the 78 nt strand. A polyclonal SAb population was assembled as described above. The polyclonal SAb population can be fractionated into individual monoclonal SAb reagents using the following procedures.

### Example 2

### Monoclonal SAb Preparation

The polyclonal SAb population is amplified by PCR to produce double stranded 78 nt and double stranded 40 nt molecules using specific primers. Amplification artifacts and PCR-errors are minimized by using polymerase with high fidelity and low number PCR cycles 1 (25 cycles). PCR products are elctrophoresized in 3½ high resolution agarose gel and 78 nucleotide fragments are recovered and purified by Qiagen Gel extraction kid. The purified 78 nt double strand DNA are cloned into PCR cloning vector (such as pGEM-T-Easy) to produce plasmid containing individual copies of the ds 78 nt fragment. The E. coli bacteria (e.g. strain JM109, Promega) are transformed with the plasmids by electroporation.

The transformed bacteria are cultured on LB/agar plates containing 100 μg/ml Ampicillin. Bacteria containing the 78 nt fragment produce white colonies and bacteria that do not contain the 78 nt fragment expresses 13 gal and form blue colonies. Individual white colonies are transferred into liquid growth media in microwells (e.g. SOC media, Promega) and incubated overnight at 37° C.

The contents of the wells are amplified after transferring an aliquot from each well into a PCR microplate. The need to purify the PCR product is avoided by using appropriate primer and PCR conditions. SAb molecules are assembled in microplates using the previously cited process of adding 40 nt-fragments and hybridization in a thermalcycler using a defined heating and cooling cycle.

### Example 3

### Analysis and Database Construction

Reactive panel profiling of monoclonal SAb clones is used to compare binding characteristics used in selecting reagent(s) for commercial application. Characteristics that are analyzed can include:

1) recognition of target ligand;

2) relative titer and affinity;

3) sensitivity;

4) specificity;

5) matrix effects;

6) temperature effects;

7) stability; and

8) other variables of commercial significance (e.g., lysis, effector function).

Standard test protocols are used and data collected from each clone is entered into a relational database.

Characterization assays transfer aliquots of assembled monoclonal SAb reagents to specific characterization plates for analysis. Affinity and titration assays compare relative affinity (Ka) and concentration of each reagent. Sensitivity assays compare the ability to detect low concentrations of the target ligand and provide an estimate of Least Detectable Dose. Specificity assays compare SAb recognition of irrelevant/undesirable ligands. Matrix interference studies evaluate the effect of anticipated matrix constituents on the binding of SAb. Temperature effects evaluate the relationship to binding. Stability identifies the most stable clones and problems requiring further evaluation. Other characteristics relevant to the anticipated application can also be evaluated using known means.

Target ligands for SAb binding include prokaryotic cells (e.g. bacteria), viruses, eukaryotic cells (e.g. epithelial cells, muscle cells, nerve cells, sensory cells, secretory cells, malignant cells, erythroid and lymphoid cells, stem cells, protozoa, fungi), proteins, prions, nucleic acids, and conjugated filterable compounds. The target ligands for SAb binding can be any ligand of sufficient size that can be retained by a filter membrane/molecular sieve.

### Example 4

### Preparation of Surrogate Antibody 87/48 to PCB Congener BZ101 Using Non-Amplifiable Stabilization Strand

Surrogate Antibody (SAb) molecules were produced using self-assembling oligonucleotide strands (87 nt+48 nt) to form a dimeric molecule having a 40 nt random specificity domain sequence with adjacent constant nucleotide sequences. Cycles of ligand binding, PCR amplification, bound/free separation, and reassembly/reannealing were used to enrich the SAb population with molecules that would bind a BSA-Adipoyl-BZ101 conjugate and the unconjugated BZ101 (2,2′,4,5,5′ pentachlorobiphenyl) hapten.

Methods

A. Forming a Library of Surrogate Antibodies:

A library of 87 nt ssDNA oligonucleotides containing a random 40 nt sequence, and FITC (F) and biotinylated (B) primers, were purchased from IDT. The 87 nt ssDNA was designated #22-40-25 (87g2) to reflect the numbers of nucleotides in the constant sequence regions flanking the variable region. The is the specificity strand of the surrogate antibody molecule and the sequence of the 87mer is shown below (top strand; SEQ ID NO: 9), while the 48 nt oligonucleotide (stabilization strand) shown is below (bottom strand; SEQ ID NO: 10).

```
5′- GTA AAA CGA CGG CCA GTG TCT C - (40 nt) -
A GAT TCC TGT GTG AAA TTG TTA TCC -
3′
        ||| ||| ||| ||| ||| ||
3′- CAT TTT GCT GCC GGT CA ggagctctcg
            ||| ||| ||| ||| ||| |||
            AGG ACA CAC TTT AAC AAT AGG -
5′
```

The two constant region nucleotide sequences on either side of the variable sequence are complementary to the nucleotide sequences of a juxtaposed 48 nt. stabilization oligonucleotide. The stabilization strand is FITC-labeled 5′- and referenced as oligonucleotide (#F21-10-17) (bases in bold are non-complimentary to bases on the 87 nt specificity strand):

Oligos were reconstituted in DI water to 0.1 mM (100 pm/μl) and stored as stock solutions in 2 ml screw top vials at −20° C. (manufacturer claim for reconstituted stability is >6 months). Working aliquots of 20 μl each were dispensed into PCR reaction tubes and stored at −20° C.

B. Selection; Cycle 1

4 μl of 0.1 mM ssDNA oligonucleotide A22-40-25 (i.e. “+87”) library (2.4×10¹⁴ molecules) were mixed with 4 μl of 0.1 mM F21-10-17 (i.e. “−40”) that is FITC-labeled at 5′ end and 2 μl of 5×TNKMg5 (i.e. TNK buffer containing 5 mM MgSO4) buffer. TNK Buffer is a Tris Buffered Saline, pH 8.0. The 5× stock comprise 250 mM Tris HCl, 690 mM NaCl, 13.5 mM KCl and a working (1×) buffer comprises 50 mM Tris HCl, 138 mM NaCl, and 2.7 mM KCl. TNK5Mg is TNK above with 5 mM MgSO₄ (1:200 dilution of 1M MgSO₄ stock) and 5×TNK5Mg is 5×TNK with 25 mM MgSO4 (1:40 dilution of 1M MgSO₄).

Annealing of SAb molecules was performed using the HYBAID PCR EXPRESS thermal cycler. The oligo mixture was heated to 96° C. for 5′, the temperature was reduced to 65° C. at a rate of 2° C./sec and maintained at this temperature for 20 min. The temperature was then reduced to 63° C. at 2° C./sec and maintained at this temperature for 3 min. The temperature was then reduced to 60° C. at 2° C./sec and maintained at this temperature for 3 minutes. The temperature was then reduced in 3° C. steps at 2° C./sec and held at each temperature for 3 minutes until the temperature reaches 20° C. Total time from 60° C. to 20° C. is 40 min. Total annealing time of 1.5 hours.

To assay for the formation of the surrogate antibody eletrophoresis was employed. On each preparative gel, a FAM-87 and F-48 was loaded to demonstrate the location of the corresponding bands and SAb. On a parallel gel (or the other half of the preparative gel), a 10 bp ladder, 48ss, 87ss and the retentate PCR product next to an aliquot (0.5 μl) of each annealed SAb. 10 μl of reaction mixture from above was mixed with 7 μl, 60% w/v sucrose. Mixture was loaded onto a 20% acrylamide gel. The 48 nt (F21-10-17) and dsSAb appeared as green fluorescent bands. The 48 band runs at approximately 50 base pairs and the dsSAb runs about 304. After extracting the Sab, the gel is stained with EtBr (1 μl of 10 mg/ml into 10 ml buffer). The 87 band will appear at approximately 157 bp, using the standard molecular weight function.

The gel fragment containing the SAB 87/48 band was excised and place in a 1.5 ml eppendorf tube. The gel fraction was macerated using a sterile pipette tip and 400 μl TNKMg5 buffer containing 0.05% v/v Tween 20 is added and the sample is then shaken on a rotating platform at the lowest speed for 2 hours/RT. The gel slurry was aspirated and added to a Pall Filter 300K and spun in Eppendorf 5417R at 1-5000×g (7000 rpm) for 3′. 40 μl TNKMg5 buffer containing 0.05% Tween was added to a volume ≦440 μl and centrifuge 3′.

The volume of filtrate is measured. RFU (relative fluorescence units) of the formed Sab was measured using a 10 μl aliquot of the filtrate and 90 μl buffer, and the Wallac VICTOR2, mdl 1420 (Program name “Fluorescein (485 nm/535 nm, 1”). A blank of buffer only was also measured. Total fluorescence was calculated by subtracting the background and multiplying by the appropriate dilution factor and volume.

1/10 volume (40 μl) MeOH was added to the filtrate along with 20 μl BSA-aa-BZ101 conjugate (1 μg/μl conjugate concentration in TNKMg5 Tw0.05 containing 10% MeOH v/v) to filtrate. The BSA-AA-BZ101 conjugate, synthesis, characterization was performed as outlined in Example 5. The sample was incubated for 1 hour/RT.

The reaction mixture was aspirated and added to a new Nanosep 100 K Centrifugal Device and centrifuge at 1000 g/3′. (The Nanosep 100K and 300K Centrifugal Devices were purchased form PALL-Gelman Cat #OD100C33 and are centrifugal filters with Omega low protein and DNA binding, modified polyethersulfone on polyethylene substrate.) The filters were used to fractionate SAb bound to BSA-AD-BZ101 from unbound Sab. SAb bound to the conjugate was recovered in the retentate while unbound SAb continued into the filtrate. The filtrate was aspirated and added to new 1.5 ml Eppendorf tube. 100 μl of mixture was removed and the RFU's was quantified in a microwell plate using Wallac Victor II. The retentate was washed only one time for cycle 1 (two times for cycle 2 and 3 times for cycles 3-6) at 1000 g/3-8′ using 400 μl aliquots of TNKMg5 buffer (without Tween and MeOH). Spin times vary from filter to filter (generally 3-8 minutes). Retentate was saved for SAb, keep filtrate and pool to measure fluorescence x volume to coincide with retentate RFU. Filtrate was discarded.

SAb (when SAb is bound to conjugate, MW>100 KD) in the retentate was recovered by adding a 100 μl aliquot of DI H₂O, swirling, and aspirating. The Total RFU's was calculated for the recovered material. Percent recovery was calculated by calculating total recovered vs. total in starting amount of SAb incubated with conjugate.

B. PCR Amplification

The DNA recovered from the retentate was amplified using a 40 cycle PCR amplification program and 2 μM of primer F22-5 and 2 uM of primer Bio21-4. Bio21-4 adds biotin to 5′ end of −87 oligonucleotide.

PCR Primers. The primers were designed to amplify only the 87 strand (the specificity strand) and not the 48 strand (the stabilization strand). This was accomplished by having 4-5 bases on the 3′ end that compliment the 87 strand but not the 48 strand. See FIG. 7. Four to five bases of non-complimentarity was sufficient to inhibit elongation.

The primer sequences used for PCR amplification were as follows. Primer F22-5—amplifies off of the −87 strand to make a new +87 and comprise the sequence: 5′ FAM-GTA AAA CGA CGG CCA GTG TCT C₃′(SEQ ID NO: 11). Primer Bio-21-4—amplifies off of the +87 to make a biotin-labeled −87 that in some embodiments can be used to extract −87 strands that do not anneal to the 48. The sequence for Bio-21-4 is 5′ bio-GGA TAA CAA TTT CAC ACA GGA ATC T 3′ (SEQ ID NO: 12).

Primers were reconstituted in 10 mM Tris (EB) to 0.1 mM (100 pm/μl) and stored in 2 ml screw top vial at −20° C. as a stock solution (claim for reconstituted stability is >6 months). Working aliquots of 20 μl were dispensed into PCR reaction tubes and stored frozen at −20° C.

PCR reaction: 10 μl of the retentate was added to a 0.2 ml PCR tube. 5 μl of Thermopol 10× buffer, 1 μl NTP stock solution (PCR dNTP, nucleotide triphosphates 10 mM (Invitrogen 18427.013) which contains a mixture of 10 mM of each of four nucleotides (A, G, C, T), 12 μL of 5M Betaine (Sigma B-0300) and 10 μl of 10 pmole/μl of each primer was added. QS to 49.5 μl with DI H₂O. The program was run with the following parameters: 3 min, 94°-65°-72° 30 sec each×35, 10° hold. When PCR machine is at 96° 5 μl of Taq DNA Polymerase ((NEBiolabs cat# MO267S) 5 U/μL) is added the reaction is mixed and placed in PCR machine.

Following the PCR reaction, 5 μL of PCR product were run on a 3% Agarose 1000 gel or 4% E-gel with controls of 10 bp ladder and ss oligos to verify amplification and size of bands. The remaining amplified DNA is purified by salt precipitation using 100% ethanol. Specifically, ⅓ volume (100 μl) of 8M Ammonium Acetate is added to 200 μl of the amplified DNA. 2.6 times the combined (DNA+Ammonium Acetate) volume (˜780-800 ul) of cold absolute ethanol (−20° C.) is added to the tube. The tube is swirled and stored on ice for 1 hr. The sample is centrifuged for 15′/14,000 g 4° C. in a refrigerated centrifuge. The supernatant liquid is removed without touching or destroying the pellet. 0.5 ml of 70% (V/V) ethanol is added. The sample is mixed gently and centrifuged for 5′/14,600 g. The supernatant is removed without disturbing the pellet and evaporate to dryness by exposing to air at RT.

When amplifying selected DNA from retentate, the following controls are also run: no DNA, 87 alone, and 48 alone. This will assure that the bands from the retentate are the right size and are not due to primer dimers. It will also show that the 48 strand is not amplifying in the SAb tube. By itself, the −48 will amplify and can be detected in the −48 control tube. This will identify the position of the ds −48 in the SAb tube if it was amplified.

Reannealing The pellet was reconstituted by adding 8 μl of a solution containing 4 μl of sterile DI H₂O+4 μl of 0.1 mM 48 nt oligonucleotide (F21-10-17). The sample was transferred to a 0.2 ml PCR tube and 2 μl of 5× TNKMg5 buffer was added. (Note; the addition of excess F21-10-17 (−48 nt) primer drives the formation of the desired +87/−48 SAb molecules).

B. Cycle 2-6: Annealing SAb

The dsSAb was annealed by heating the reconstituted material in a 0.2 ml PCR tube using the temperature program previously specified for annealing. After the first cycle, multiple bands appear. Thus a parallel SAb aliquot was run with its corresponding PCR starting strands to verify that the band being cut out is in fact the new SAb. To verify that the SAb band was ds 87/48, an aliquot was removed and run on a denaturing gel (16%, boiling in 2× urea sample buffer) to verify that the band from the preparative gel contains both 87 and 48 strands.

Electrophoresis was performed at 120 v for 40 min. 7 μl of 60% w/v sucrose was mixed with 10 μl of DNA and the sample is loaded. Any DNA component with FITC at 5′ end (i.e. SAb 87/48, ds 48 and ss48) will appear on the gel as a green fluorescent band under long wavelength. Run 5 pMol of F21-10-17 (−48 nt primer) in an available lane as a size marker. SAb will be observed to co-migrate with 250-300 nt dsDNA in 20% acrylamide native gel. The SAb-gel section was excised and macerated in 250 μl of TNKMg5 Tw0.05 buffer. The sample was incubated for 2 hrs/RT while agitating on rotating platform at the lowest speed.

The gel suspension was transferred to a Pall 300K Centrifugal Device and centrifuge at 1-5000 g/3′ to remove the polyacrylamide. The retentate was washed by adding a 50 μl aliquot of buffer, centrifuge at 1000 g/3′. The SAb is recovered from the filtrate for use in subsequent selection cycle.

The RFU's of SAb and buffer blank was measured as describe above using a 100 ul aliquot of the filtrate on the Wallac Victor2.

C. Selection Cycles 2-7

1/10 volume of MeOH was added and 20 μl BZ101-aa-BSA (1 μg/μl) as in cycle 1. The sample was incubated for 1 hr and selected using Pall 100 K filter. RFU measurement of the retentate after 2 washes for cycle 2 and 3 washes for cycle 3-6 were taken. Subtraction of the background RFU allow the determination of the % recovery.

Negative Selection. In this example, negative selection using BSA was not performed in Cycle #1-6.

When negative selection was desired, 250 μl of SAb 87/48 filtrate (2-20 pMol by FITC) was mixed with 20 μl of a 1 μg/μl (20 μg) BSA solution. The sample is Incubated for 30′/RT. The RFU's was measured in 100 ul aliquot using Wallac VICTOR II Program.

250 ul of the above reaction mix (20 μl is saved for 16% non-denaturing PAGE and 8% denaturing PAGE with 8M urea) was added to Nanosep 100K Centrifugal concentrator. The filter was centrifuged at 1000 g/15′/RT. Total volume in filtrate was ˜240 μl. Aspirate filtrate and place in new 1.5 ml Eppendorf tube. RFU's of 100 μl aliquot were checked.

The filter was washed by adding 200 μl TNKMg5 buffer, centrifuge (1000 g/10′/RT), add additional 200 μl of same buffer after centrifugation, re-centrifuge, add 100 μl of same buffer and centrifuge again. 100 μl DI H₂O was added, filtered, swirled and aspirate retentate. RFU's were determined on Wallac VICTOR II of SAb bound to BSA by aspirating retentate and % recovery was determined.

200 μl of negatively selected filtrate was mixed with 20 μl (1 μg/μl) of the BSA-aa-BZ10 conjugate suspended in TNKMg5 buffer. The mixture was incubated for 1 hour/RT with a total volume of 220 μl. The reaction solution was added to a new Nanosep 100K centrifugal device and centrifuged at 1000 g/3′. A wash was performed 3 times using a TNKMg5 buffer. Measure RFu's of a 100 μl aliquot of the filtrate to determine % of unbound (free) SAb.

100 μl of DI H₂O was added to filter, swirled, and the retentate was aspirated. The entire sample was placed in a microtiter plate well. RFU's of sample were measured and background and calculate % Recovery.

Additional Steps. 1-20% of the bound SAb recovered in the 100 μl aliquot was used for PCR amplification with primer. This will again generate dsDNA in 4 tubes each containing 50 μl, as described previously. Cycles of negative and positive selection were repeated until no further enrichment in % recovery was observed in the SAb population.

Additional cycles can be performed by preincubating the free hapten with the polyclonal SAb library prior to addition of the conjugate, and collecting the filtrate for subsequent amplification. A cycle(s) of affinity enhancement can be performed by incubating the SAb and conjugate in the presence of elevated MeOH, surfactant, decreased pH, and/or increased salt. High affinity SAb remaining bound to the conjugate is amplified. The process of Polyclonal SAb production proceeds through 1. Binding, 2. Specificity Enhancement, 3. Affinity Enhancement, prior to production of monoclonal SAb clones.

Calculations. The total amount of RFU's in the recovered conjugate-binding aliquot vs. the total amount of RFU's that were present when incubated with the conjugate was determined. For negative selection; the amount of RFU's in the recovered BSA-binding aliquot vs. the total amount of RFUs present when incubated with BSA was determined. RFUs quantified from filtrate provides supportive data and information indicating unbound SAb and loss on filter device.

Notes: The DNA/conjugate and DNA/BSA ratios in cycles #2-5 was 10-100 nM DNA/2,000 nM protein, or 1 molecule of SAb to 20-200 molecules of the conjugate or BSA. This calculation assumes that the conjugate has the reported 20 moles of BZ101 per mole of protein). The molecular weight of the (SAb 87/48-BSA-aa-BZ101) complex=(A22-40-25=27.4 Kd)+(FM21-10-17=15.4 Kd)+(BSA=67 Kd)+(20 BZ101=7 Kd). Total=˜116.8 Kd; 2SAb:1 Conjugate ˜159.6 Kd.

### Example 5

### Preparation of Surrogate Antibody 78/48 to PCB Congener BZ101

Surrogate Antibody (SAb) molecules were produced using self-assembling oligonucleotide strands (78 nt+48 nt) to form a dimeric surrogate antibody molecule having a 40 nt random sequence binding loop with adjacent constant nucleotide sequences. Cycles of ligand binding, PCR amplification, bound/free separation, and reassembly/reannealing were used to enrich the SAb population with molecules that would bind a BSA-Adipoyl-BZ101 conjugate and the unconjugated BZ101 (2,2′,4,5,5′ pentachlorobiphenyl) hapten.

A. Background

PCBs are chlorinated aromatic compounds that can exist in 209 different molecular configurations (congeners). The higher chlorinated species are relatively stable to oxidation at elevated temperatures, and were used as heat transfer agents from 1929 to 1977. During this period 1.4 billion pounds were produced and commercialized as mixed congener Aroclor® products, named to reflect their 12 carbon biphenyl nucleus and average percentage of chlorine (e.g. Aroclor 1242, 1248, 1254, etc.). Today these compounds are ubiquitous environmental contaminants, having been used in transformers, industrial machinery and household appliance capacitors, compressors, paint, insulation, adhesives, and chemical processing equipment. The Toxic Substances Control Act (TSCA) of 1976 established the legal framework for their elimination, but prior pollution, new spills, and the continuing disposal of contaminated materials persist. PCBs have been classified as Persistent Organic Pollutants (POPs) and efforts are underway to draft an international treaty that would coordinate their elimination.

Polychlorinated biphenyls (PCBs) have been classified as endocrine disrupters. They mimic estrogens (xenoestrogens) and upset endocrine hormone balance. Male sexual development is dependent upon androgens, and imbalances in the androgen/estrogen ratio caused by PCBs are thought to interfere with genital development. PCBs are linked to neuro-developmental defects in utero and concern exists regarding fetal health in mothers that consume PCB-contaminated fish. PCBs have also been found in breast milk, a significant source of exposure for neonates. Studies have shown that pre-natal exposure to PCBs causes mental and physical abnormalities. Other effects are lower birthing weight, altered thyroid and immune function, and adverse neurological effects. Other studies suggest that persistent exposure of newborns to PCBs results in hypoandrogenic function in adult males (Kim et al. (2001) Tissue Cell 33:169-77).

A health effect of particular concern is the neurotoxicity caused by PCB-altered thyroid function during the critical period of thyroid-dependent brain development. This period extends from pre-partum to 2 years postpartum. Thyroid function regulates the assembly and stability of the cytoskeletal system required for neuronal growth, and the development of the cholinergic and dopaminergic systems of the cerebral cortex and hippocampus. Exposure to PCBs causes enlargement of the thyroid with an accompanying reduction in circulating thyroxine (T4) levels. The likely cause is the structural similarity that exists between selected congeners and the thyroid hormone, and the ability of PCBs to be bound by transport proteins such as transthyretin with high affinity. PCBs have been shown to act as agonists and antagonists when bound to thyroid receptors. The neurological effects resulting from thyroid disorders, and those reported following PCB or dioxin exposure, bear a striking similarity and suggest a common mechanism.

Three congeners (BZ138, 153, 180) listed in the EPA reference method, interfere with sexual hormone regulation by competing with the natural ligand for binding to two nuclear receptors. These congeners also have different affinities for estrogen and androgen receptors and can induce both cell proliferation (nM) and inhibition (μM). PCBs are suspected agents in the development of endometriosis, have been shown to be immunosuppressive, and can be carcinogenic, Carcinogenesis is believed to be mediated through binding to the Ah receptor (aryl hydrocarbon) via the same pathway described by Poland and others for dioxins.

The surrogate molecules of the invention being developed for the PCB array combine attributes of aptamers and natural antibodies. These molecules are of nucleic acid composition and retain a stable secondary structure having constant regions and a hydrophobic binding cavity. Pre-formed and sequentially enriched libraries of molecules having a random assortment of binding-cavity sequences are fractionated to amplify those that bind the target. A monoclonal antibody procedure will produce homogenous molecules for characterization, identification, sequencing and synthesis. The preparation process is expected to significantly reduce the time of development. The molecule has been designed to permit the simple attachment of multiple labels. Animals are not used, and induction of an immune response is not required. Production is by PCR or direct synthesis. The surrogate antibody molecules facilitate the elimination of PCBs from the environment and remove a persistent public health pathogen.

B. Materials and Methods

I. Selection: Cycle 1

Forming the surrogate antibody: The library of surrogate antibodies used in the following experiment was formed as follows. A library of 78 nt ssDNA oligonucleotides containing a random 40 nt sequence, and FITC (F) and biotinylated (13) primers, were purchased from Gibco-Invitrogen life technologies. The 78 nt ssDNA was designated #17-40-21 to reflect the numbers of nucleotides in the constant sequence regions flanking the variable region. The sequence of the 78mer (i.e., the specificity strand; SEQ ID NO: 13) is shown below along with the 48 nt oligonucleotide (i.e., the stabilization strand; SEQ ID NO: 14).

(78 nt oligonucleotide, shown as top strand)
5′ GTA AAA CGA CGG CCA GT (40 nt) -
TCC TGT GTG AAA TTG TTA TCC 3′
||| ||| ||| ||| ||| ||
3′ CAT TTT GCT GCC GGT CA ggagctctcg
||| ||| ||| ||| ||| ||| |||
AGG ACA CAC TTT AAC AAT AGG 5′
(48 nt oligonucleotide shown as bottom strand)

The two constant region nucleotide sequences on either side of the variable sequence are complementary to the nucleotide sequences of a juxtaposed 48 nt stabilization oligonucleotide. The bases in bold of the FITC-labeled 5′-oligonucleotide (#F21-10-17) are non-complimentary to bases on the 78 nt strand. Oligos were reconstituted in DI water to 0.1 mM (100 pm/μl) and stored as stock solutions in 2 ml screw top vials at −20° C.

4 μl of 0.1 mM ssDNA oligonucleotide A17-40-21 (i.e. “+78”) library (2.4×10¹⁴ molecules) (i.e., specificity strand) was mixed with 4 μl of 0.1 mM F21-10-17 (i.e. “−40”) (stabilization strand) that is FITC-labeled at 5′ end and 2 μl of 5×TNKMg5 (i.e. TNK buffer containing 5 mM MgSO4) buffer. TNK Buffer is Tris Buffered Saline, pH 8.0 (a 1× stock comprises 50 mM Tris HCl 138 mM NaCl and 2.7 mM KCl). The TNKMg5 buffer comprises the TNK buffer plus 5 mM MgSO₄.

SAb molecules were annealed using the HYBAID PCR EXPRESS thermal cycler (program name: “Primer”). The oligo mixture is heated to 96° C. for 5′, the temperature is reduced to 65° C. at a rate of 2° C./sec and maintained at this temperature for 20 min. The temperature was then reduced to 63° C. at 2° C./sec and maintained at this temperature for 3 min. The temperature was then reduced to 60° C. at 2° C./sec and maintained at this temperature for 3 minutes. The temperature was then reduced in 3° C. steps at 2° C./sec and held at each temperature for 3 minutes until the temperature reaches 20° C. Total time from 60° C. to 20° C. is 40 min.

10 μl of reaction mixture from above was mixed with 7 μl, 60% w/v sucrose and loaded onto a 1 mm 16% acrylamide gel (19:1 ratio Acrylamide:Methylene Bisacylamide). The gel was examined using long wave UV-366 nm BLAK-RAY LAMP model UVL-56. The 40 nt (F21-10-17) and dsSAb appear as green fluorescent bands.

The “SAb 78/48” band was excised from the gel and the gel fraction was mascerated in 400 μl TNKMg5 buffer containing 0.05% v/v Tween 20. The gel slice was then shook on a vortex at the lowest speed for 2 hours/RT.

The gel slurry was aspirated and the gel suspension is added to an Amicon (Microcon) Centrifugal Device and spin at 1000 g/10′. 40 μl TNKMg5 buffer containing 0.05% Tween was added and the sample was centrifuge at 1000 g/10′. Total volume ≦440 μl.

40 μl MeOH was added to the filtrate. To quantify the amount of antibody, RFU (relative fluorescence units) was measured using a 100 μl aliquot of the filtrate and the Wallac VICTOR2, mdl 1420 (Program name “Fluorocein (485 nm/535 nm, 1”).

All of the SAb filtrate was added to the Nanosep 100K Centrifugal Device (Pall-Gelman) and it was Centrifuge at 1000 g/15′. RFU was quantified using a 100 μl aliquot of the filtrate as above.

II. Selection of Surrogate Antibody

The filtrate from above is added to a 0.2 ml PCR tube containing 20 μl BSA-aa-BZ101 conjugate (1 μg/μl conjugate concentration) in TNKMg5 Tw 0.05 containing 10% MeOH v/v). BSA-AA-BZ101 conjugate was synthesized as described below. Methanol added to 10% v/v final concentration. Tween 20 was added to 0.05% w/v final concentration. The sample was incubated for 1 hour/RT.

The reaction mixture was aspirated and added to new Nanosep 100K Centrifugal Device and centrifuge at 1000 g/10′. The Nanosep 100K Centrifugal Devices (Cat #OD100C33 PALL-Gelman, centrifugal filter with Omega low protein and DNA binding, modified polyethersulfone on polyethylene substrate) used was able to fractionate SAb bound to BSA-AD-BZ101 from unbound SAb. SAb bound to the conjugate was recovered in the retentate while unbound SAb continued into the filtrate. The filtrate was aspirated and added to new 1.5 ml Eppindorf tube. 100 μl was taken and the RFU's were quantified in a microwell plate using Wallac Victor II. The retentate was washed 3 times at 1000 g/10′ using 200 μl aliquots of TNKMg5 buffer (sans tween and MeOH). The filtrate was discarded.

SAb (when SAb is bound to conjugate, MW>100 KD) in the retentate was recovered by adding a 100 μl aliquot of DI H₂O, swirling, and aspirating. The Total RFU's was calculated for the recovered material. % recovery was determined by calculating total recovered vs. total in starting amount of SAb incubated with conjugate.

III. PCR Amplification

The DNA recovered from the retentate was amplified using a 40 cycle PCR amplification program and 2 μM of primer FM13-20 and 2 uM of primer BioM13R48. BioM13R48 adds biotin to the 5′ end of +78 oligonucleotide. The PCR reaction amplifies +78 nt, −48 nt, −78 nt and +48 nt strands thereby reducing the theoretical yield of SAb

The primer sequences used for the PCR amplification are as follows: Primer #FM13-20 (SEQ ID NO: 15) has the sequence 5′ FITC-GTA AAA CGA CGG CCA GT 3′ were FITC is fluorocein isothiocyanate and Primer #BioM13R48 (SEQ ID NO: 16) has the sequence 5′ Bio-GGA TAA CAA TTT CAC ACA GGA 3′ where Bio is biotin. The primers were reconstituted in DI water to 0.1 mM (100 μm/μl) and stored in 2 ml screw top vial at −20° C. as a stock solution.

100 μl of the retentate was added to a 0.2 ml PCR tube. 20 μl of Thermopol 10× buffer, 4 μl NTP stock solution, and 4 μl of 100 pmole/μl of each primer was added. The final volume was brought to 200 μl with DI H₂O. The samples were mixed and placed in PCR machine. When the temperature reaches 96° C. the program was pauses and 2 μl Deep Vent (exonuclease negative) DNA Polymerase stock solution (2 units/μl) (New England BioLabs cat #MO 259S) was added with 10× ThermoPol Reaction Buffer. 10× ThermoPol buffer comprises 10 mM KCL, 10 mM (NH4)₂SO₄, 20 mM Tris-HCL (pH8.8, 2° C.), 2 mM MgSO4, and 0.1% Triton X-100. The reaction mixture was aliquoted into empty 50 μl PCR tubes preheated in the machine to 96° C. The total amplification time was about 2.5-3 hours.

The amplified DNA was purified by extraction with an equal volume of a phenol-chloroform-isoamyl Alcohol solution (25:24:1 v/v). 200 μl of the amplified DNA was transferred to a 1.5 ml Eppindorf tube. 200 μl of the extraction solution was added to the tube. The tube was swirled and then centrifuged for 5′/12,000 g. The supernatant (buffer layer) was aspirated and transferred to a new 1.5 ml Eppindorf tube.

The aspirated DNA solution undergoes salt precipitation using 100% ethanol. 100 μl of 8M Ammonium Acetate was added to ˜200 μl of the aspirated DNA. 2.6 times the combined (DNA+Ammonium Acetate) volume (˜780-800 μl) of cold absolute ethanol (−20° C.) was added to the tube. The tube was mixed and store in ice water for 30′. The sample was centrifuged for 15′/12,000 g. The supernatant was aspirated and discarded. 0.5 ml of 70% (V/V) ethanol was added and the sample was centrifuged for 5′/12,000 g. The supernatant was removed without disturbing the pellet and evaporate to dryness by exposing to air at RT. The pellet was reconstituted by adding 8 μl of a solution containing 4 μl of sterile DI H₂O+4 μl of 0.1 mM primer (F21-10-17). The sample is transferred to a 0.2 ml PCR tube and 2 μl of 5×TNKMg5 buffer is added. The surrogate antibody was reformed by the addition of excess F21-10-17 (−48 nt) primer favors the formation of the desired +78/−48 SAb molecules.

IV. Annealing the SAb

The dsSAb was annealed by heating the reconstituted material in a 0.2 ml PCR tube using the temperature program previously specified for annealing. 7 μl of 60% w/v sucrose with 10 μl of DNA and load sample onto a 16% acrylamide gel. Any DNA component with FITC at 5′ end (i.e. SAb 78/48, ds 48 and ss48) will appear on the gel as a green fluorescent band under long wavelength (UV-366 nm BLAK-RAY LAMP model UVL-56). The 5 pMol of F21-10-17 (−48 nt primer) was also run on the gel as a size marker. The SAb 78/48 will be observed to co-migrate with 500-600 nt dsDNA. The SAb-gel section was excised and mascerated and 250 μl of TNKMg5 Tw 0.05 buffer was added to the sample. The sample was then incubated for 2 hrs/RT while agitating on vortex at the lowest speed.

The gel suspension was transferred to an Amicon PCR Centrifugal Device and centrifuge at 1000 g/10′ to remove the polyacrylamide. The retentate was washed by adding a 50 μl aliquot of buffer, centrifuge at 1000 g/10′. The recovered SAb from the filtrate for use in subsequent selection cycle. The Sab was quantified by FU's using a 100 μl aliquot of the filtrate on the Wallac Victor2.

V. Selection Cycles 2-7

Negative selection using BSA was not performed in Cycle #1. The negative selection mixture comprises 250 μl of SAb 78/48 filtrate (2-20 pMol by FITC) with 20 μl of a 1 μg/μl (20 μg) BSA solution. The sample was incubate for 30′/RT and the FU's of 100 μl aliquot using Wallac VICTOR II was measured.

250 μl of the above reaction mix (20 μl is saved for 16% non-denaturing PAGE and 8% denaturing PAGE with 8M urea) is added to Nanosep 100K Centrifugal concentrator. The filter was centrifuged at 1000 g/15′/RT. The total volume in filtrate was ˜240 μl. The filtrate is aspirated and place in a new 1.5 ml Eppindorf tube. The RFU's of a 100 μl aliquot was determined.

The filter was washed by adding 200 μl TNKMg5 buffer, centrifuge (1000 g/10′/RT), and an additional 20011 of same buffer was added after centrifugation. The sample was re-centrifuged and 100 μl of same buffer was added. The sample was centrifuged again. 100 μl DI H₂O was added to filter and swirled and the retentate is aspirated. The RFU's was determined on Wallac VICTOR II of SAb bound to BSA by aspirating retentate and determining % recovery.

200 μl of negatively selected filtrate was mixed with 20 μl (1 gμ/μl) of the BSA-aa-BZ10 conjugate suspended in TNKMg5 buffer. The sample was incubated for 1 hour/RT. Total volume of the reaction is 220 μl.

The reaction solution was added to a new Nanosep 100K centrifugal device and centrifuged at 1000 g/15′. The filter was wash 3 time using TNKMg5 buffer. RFU's of a 100 μl aliquot of the filtrate was determined along with the % of unbound (free) SAb.

100 μl of DI H₂0 was added to the filter, swirled, and the retentate aspirated. The entire sample was placed in a microtiter plate well and the RFU's and % recovery was measured.

From 1-20% of the bound SAb recovered in the 100 aliquot for PCR amplification was used with primer #BioM13R48 (100 pMol) and FM13-20 (100 pMol). This will again generate dsDNA in 4 tubes each containing 50 μl as described previously. Cycles of negative and positive selection are repeated until no further enrichment in % recovery is observed in the SAb population.

Additional cycles can be performed by preincubating the free hapten with the polyclonal SAb library prior to addition of the conjugate, and collecting the filtrate for subsequent amplification. A cycle(s) of affinity enhancement can be performed by incubating the SAb and conjugate in the presence of elevated MeOH, surfactant, decreased pH, and/or increased salt. High affinity SAb remaining bound to the conjugate was amplified. The process of Polyclonal SAb production proceeds through 1) binding, 2) specificity enhancement, and 3) affinity enhancement prior to production of monoclonal SAb clones.

VI. Calculations

The total amount of RFU's in the recovered conjugate-binding aliquot vs. the total amount of RFU's that were present when incubated with the conjugate represents the % of the surrogate antibody bound.

For negative selection, the amount of RFU's in the recovered BSA-binding aliquot vs. the total amount of RFUs present when incubated with BSA is determined.

Additional calculations include RFUs quantified from the filtrate that provides supportive data and information indicating unbound SAb and loss on filter device.

Further note that the DNA/conjugate and DNA/BSA ratios in cycles #2-5 was 10-100 nM DNA/2,000 nM protein, or 1 molecule of SAb 78/48 to 20-200 molecules of the conjugate or BSA. This calculation assumes that the conjugate has the reported 20 moles of BZ101 per mole of protein. In addition, the molecular weight of the (SAb 78/48-BSA-aa-BZ101) complex is about 113.4 Kd (A17-40-21=24 Kd)+(FM21-10-17=15.4 Kd)+(BSA=67 Kd)+(20 BZ101=7 Kd). The molecular weight of 2SAb:1 conjugate is ˜152.8 Kd and the molecular weight of 1SAb:2 conjugate ˜189.4 Kd.

C. Results

The production of surrogate antibody show in FIG. 1 was initiated to provide a more versatile core molecule than an aptamer having a stem-loop structure. The design incorporates constant region domains that bracket binding specificity domain. The multi-oligonucleotide structure allows for the simple attachment of multiple labels (e.g. FITC, biotin) that may, or may not be the same. Multiple, self-directed and self-forming, binding cavities can be readily incorporated. A stabilizing strand that is separate from the binding strand offers a convenient site for chemical modifications when required.

The surrogate antibodies are formed by annealing a “specificity-strand” to a “stabilizing-strand” prior to incubation with the target. Molecules that bind are amplified using asymmetric PCR that preferentially enriches the “specificity-strand”. The constant sequence “stabilizing-strand” is added, and surrogate molecules are annealed for another selection cycle.

Surrogate antibodies can be assembled using “binding strands” that vary in the number of nucleotides in the binding loop. Each of these molecules will have a different binding cavity size and unique binding configurations. FIG. 8 illustrates the electrophoretic mobility of the surrogate antibodies that were assembled using different combinations of “specificity” and “stabilizing” primers. Fluorocein-labeled “stabilizing strands” (prefix “F”) and un-labeled “specificity strands” (prefix “A”) were used in the production of these molecules. This combination illustrates a significant shift in the electrophoretic mobility of the fluorocein-labeled “Stabilization” strand and the annealed molecule. The lanes in FIG. 8 are as follows: Lane 1 primer A78, Lane 2 primer F40, Lane 3 surrogate antibody, “A58/F40”, Lane 4 surrogate antibody “A58/F48” Lane 5 surrogate antibody “A88/F40”, Lane 6 surrogate antibody “A88/F48”, Lane 7 primer F48, Lane 8 primer A88, Lane 9 surrogate antibody “A78/F40”, Lane 10 surrogate antibody “A78/F48”, Lane 11 surrogate antibody “A78/F40, Lane 12 dsDNA markers (number of nucleotides in each strand indicated to right), Lane 13 primer F40.

The surrogate antibodies that were characterized using non-denaturing acrylamide gel electrophoresis were re-characterized using a denaturing gel (8% acrylamide, 8M urea) to verify the duplex nature of the molecule and approximate 1:1 stoichiometry of the “specificity” and “stabilization” strands (FIG. 9). The lanes in FIG. 9 are as follows: Lane 1 A78/F40, Lane 2 A78/F48, Lane 3 A78/F40, Lane 4 Primer F48, Lane 5 A88, Lane 6 F48, Lane 7 A88/F48, Lane 8 A88/F40, Lane 9 A58/48, Lane 10 A58/F40, Lane 11 F40, Lane 12 A78.

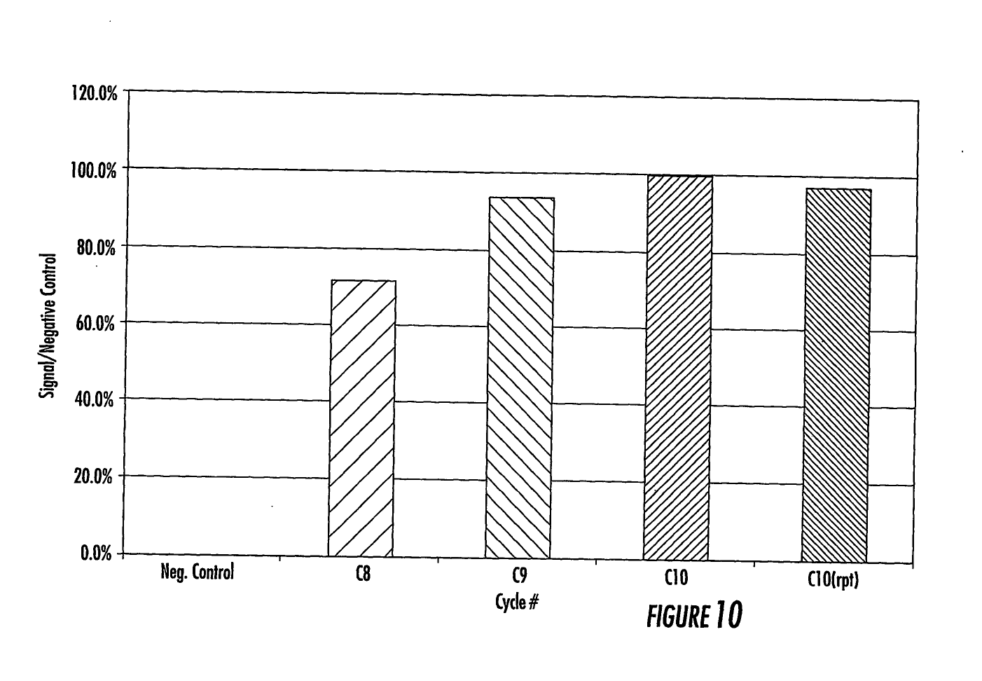

FIG. 10 illustrates the selection and enrichment of the surrogate antibodies to the BSA-PCT (BZ101 congener) conjugate through 8, 9 and 10 cycles. Signal/Negative control represents as a percent the amount of surrogate antibody bound to the target verses the amount of surrogate antibody recovered when the target is absent (negative control).

D. Observations and Conclusions

The surrogate antibody binding affinity for the non-polar BZ101 congener is believed to be the result of the binding loop/cavity designed into the molecules and hydrophobic interactions. The observation is similar to other experiments that illustrated the high affinity binding of PCB congeners by β cyclodextrins. The better than expected sensitivity obtained may also suggest the cooperative effect of hydrophobic, hydrogen, electrostatic and Van der Waals bonds. The binding of the BZ101-BSA conjugate, and the effective inhibition of binding induced by relatively low concentrations of free BZ101, was of special interest. The data suggests limited preferential binding of the conjugated ligand that was used during selection, and that the same bridge chemistry could be used in a reporter molecule for final immunoassay. This is typically not an available option when developing a hapten-specific immunoassay, where preferential antibody binding, and decreased assay sensitivity, would occur if the reporter molecule and immunogen shared the same bridge chemistry. The observation illustrates the versatility of the selection method and ability to eliminate bridge and carrier binding molecules from the SAb library. The data demonstrates the rapid production of a new binding reagent that could preferentially bind an EPA-specified PCB congener at a concentration below the regulatory action limit.

### Example 7

### Use of Surrogate Antibodies in Arrays

Five monoclonal surrogate antibody reagents to the congeners designated in Table 1 will be prepared for the Aroclor® immunoassay array.

TABLE 1
5 Congeners of Interest

| | | | M.W. |
|---|---|---|---|
| 2,2′3,4,4′5,5′ Heptachlorobiphenyl | BZ180 | C₁₂H₃C₁₇ | 395.35482 |
| 2,3,3′,4′,6 Pentachlorobiphenyl | BZ110 | C₁₂H₅C₁₅ | 326.4567 |
| 2,2′4,5,5′ Pentachlorobiphenyl | BZ101 | C₁₂H₅C₁₅ | 326.4567 |
| 2,3′4,4′ Tetrachlorobiphenyl | BZ66 | C₁₂H₆C₁₄ | 292.00764 |
| 2,2′5 Trichlorobiphenyl | BZ18 | C₁₂H₇C₁₃ | 257.55858 |

Five immunoassays, each targeting one of the Method 8082-specified congeners, will be developed. The unique response profile produced by the five tests will be used to identify the Aroclor present. The composite signal generated will be used to quantify Aroclor® concentration. A single well “total PCB” assay will be formulated using a polyclonal reagent from the five monoclonal surrogate antibodies produced.

Proposed Test Characteristics:

Aroclor® composition data published by Frame (Frame et al. (1997) Anal. Chem 468A-475A) and EPA Region V (Frame et al. (1996) J. High Resol. Chromatogr 19:657-688) were used to select target congeners that would collectively provide a unique, predictable, and detectable response profile. Table 2 illustrates the weight % composition of the congeners in each of five EPA-specified Aroclors®.

TABLE 2
Weight % Composition of Selected Congener in Five Aroclors ®

| | Congener Wt. % in Designated Aroclor | | | | |
|---|---|---|---|---|---|
| | 180 | 110 | 101 | 66 | 18 |
| molecular weight | 395.35 | 326.46 | 326.46 | 292.01 | 257.56 |
| 1260 | 11.38 | 1.33 | 3.13 | 0.02 | 0.05 |
| 1254 (composite) | 0.55 | 8.86 | 6.76 | 2.29 | 0.17 |
| 1248 (composite) | 0.12 | 2.76 | 2.06 | 6.53 | 3.79 |
| 1242 | 0.00 | 0.83 | 0.69 | 3.39 | 8.53 |
| 1016 | 0.00 | 0.00 | 0.04 | 0.39 | 10.86 |

Table 3 illustrates the molar concentration of each congener when the total Aroclor® concentration in a sample is 10 ppm, the EPA-OSWER regulatory action level for solid-waste.

TABLE 3
Molar concentration of congeners in a
sample when total Aroclor ® concentration
of the sample is 10 ppm.

| | Molar Concentration of Congener in Sample when | | | | |
|---|---|---|---|---|---|
| | Total Aroclor Concentration In Sample = 10 ppm | | | | |
| | 180 | 110 | 101 | 66 | 18 |
| 1260 | 2.88E−06 | 4.07E−07 | 9.59E−07 | 6.85E−09 | 1.94E−08 |
| 1254* | 1.38E−07 | 2.71E−06 | 2.07E−06 | 7.83E−07 | 6.41E−08 |
| 1248* | 2.91E−08 | 8.45E−07 | 6.29E−07 | 2.24E−06 | 1.47E−06 |
| 1242 | 0.00E+00 | 2.54E−07 | 2.11E−07 | 1.16E−06 | 3.31E−06 |
| 1016 | 0.00E+00 | 0.00E+00 | 1.23E−08 | 1.34E−07 | 4.22E−06 |

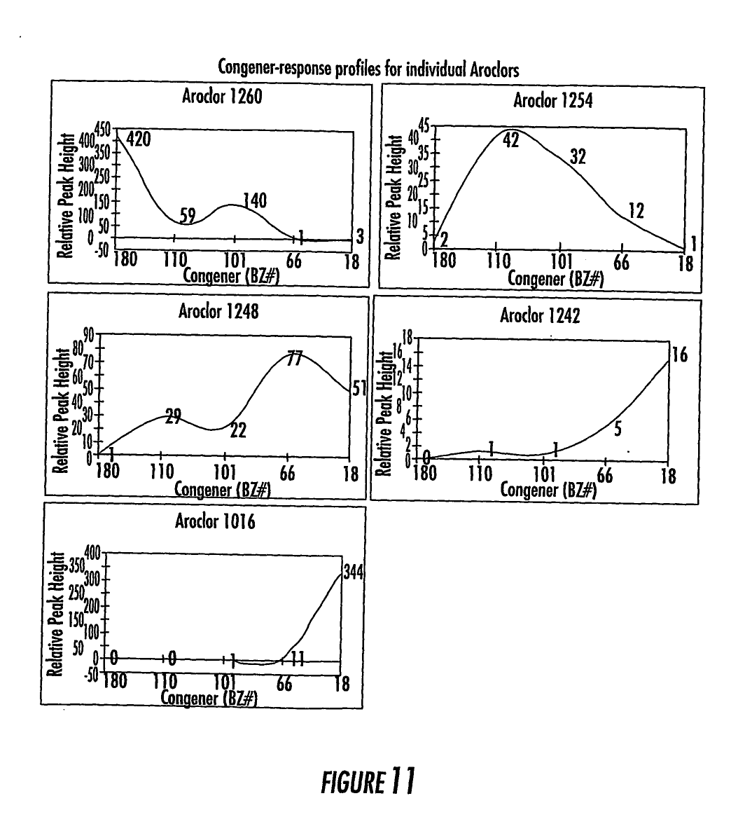

This concentration approximates the Ka each of the immunoassays and surrogate antibody would need to achieve to detect the congener in the middle (B₅₀) of their respective dose-response curves. Some of the cited applications for the test will require a practical quantitation limit of 2 ppm, a concentration that would require 2-4 times greater affinity. Based upon the BZ101 immunoassay data and the literature cited for the affinity of aptamers, immunoassays developed using surrogate antibodies should achieve the required practical detection limits without additional pre-analysis concentration steps. Table 4 indicates the relative distribution of the selected congeners in each of the Aroclors®, and FIG. 11 illustrates the unique congener response profiles the array would produce for selected Aroclors®.

TABLE 4
Relative Peak Heights of Congeners in Specified Aroclors ®

| | Ratio of Peak Heights at 10 ppm Aroclor Concentration | | | | |
|---|---|---|---|---|---|
| | 180 | 110 | 101 | 66 | 18 |
| 1260 | 420 | 59 | 140 | 1 | 3 |
| 1254* | 2 | 42 | 32 | 12 | 1 |
| 1248* | 1 | 29 | 22 | 77 | 51 |
| 1242 | 0 | 1 | 1 | 5 | 16 |
| 1016 | 0 | 0 | 1 | 11 | 344 |

*average of “a” and “g”

Surrogate Antibody Development:

The five congeners identified in table 1 for surrogate antibody development were selected on the basis of;

1. concentration compatible with the anticipated surrogate antibody binding constant (note; the sample processing chemistry developed would allow the PCBs to be concentrated and thereby overcome a disparity between binding Ka and required assay detection range.)

2. unique Aroclor® distribution profile (note; the unique response profile of the immunoassays will be used to Aroclors® in the way the gas chromatography reference method is used)

3. their citation in EPA reference Method 8082

4. congeners having an approximately equal concentration in Aroclor 1248a and 1248g, and 1254a and 1254g (note; the first generation product will not differentiate these sub-populations)

Surrogate antibody molecules will be assembled before each selection cycle into duplex oligonucleotides having one strand that is may be unlabeled or labeled using a biotin-primer, and the other strand labeled with fluorocein isothiocyanate (FITC) at the 5′ end (Kato et al. (2000) NAR 28:1963-1968). A Wallac Victor 2 multi-label reader will be used to quantify the concentration of the FITC-labeled strand and assembled SAb. Non-denaturing acrylamide gel (16%) will be used to confirm the assembly of SAb's by noting the change in mobility of the unannealed vs. annealed FITC-labeled strand. Electrophoresis using 8% acrylamide gel and 8M urea will be used to confirm that the identity of the annealed duplex molecule. Yield and % recovery of the assembled SAb will be quantified by determining the amount of SAb related fluorescence in an excised SAb gel fraction to the total fluorescence of the components.

The initial unselected population will be incubated with a congener-BSA conjugate to produce an amplified binding population. The “size-exclusion” filtration method, using the Microcon® device will be used to separate SAb molecules bound to the conjugate from those not bound. Unbound molecules will pass into the filtrate. Volume and fluorescence will be quantified and the fraction discarded. Molecules in the retentate will similarly be quantified for volume and fluorescence and then used for PCR amplification. The relative amount of fluorescence in the retentate vs. total starting fluorescence will be calculated as % recovery (%/bound/total).

PCR will be performed using two primers, one labeled with FITC. The FITC primer will be used to produce the positive congener-binding strand. Standard PCR will be performed using 40 cycles of amplification, Deep-Vent® polymerase (exonuclease free), and NTPs. PCR products will be purified with phenol/chloroform extraction and NaAc:EtOH precipitation to remove proteins (e.g. polymerase) and to concentrate the product. The “Stabilizing” primer (with/without biotin) will be added to the “binding” strand of the purified PCR pellet at a 4-10 molar excess concentration. The mixture will be annealed using a thermal cycler at 95° C./5′, 65°/20′, 60°/5′, 55°/5′, and then cooled to RT at the rate of 1°/1′. The 65° C. annealing temperature is used to favor the formation of duplex SAb's that have Tm's in the 80° C. range. Sucrose buffer (7 μl, 60%) will be added to the SAb's to increase density prior to electrophoresis. Non-denaturing electrophoresis (16% acrylamide, 100V, RT) will be used to fractionate the SAb from other components. The FITC-labeled SAb will be located on the gel by fluorescent scanning and mobility (Rf) and excised for use in selection. SAb will be extracted from the macerating gel after the addition of a buffer, incubation for 2 hours, and Microcon® filtration.

The congener-BSA conjugate will first be filtered through a Microcon® column. Conjugate appearing in the filtrate will be discarded and conjugate in the retentate recovered for use in the selection. The processed conjugate (10-20 μl) will be incubated with the purified SAb and incubated at RT/60′. The incubated solution will be filtered and SAb in the retentate recovered, quantified for FITC, and amplified. The % bound/total SAb will again be calculated. Incubation with exonuclease I will be used to demonstrate the formation and use of the duplex structure (note; SAb molecule should be resistant to degradation by this enzyme). Selection cycles will continue until further enrichment in % B/T is not produced.

Specificity enrichment will remove surrogate antibodies that recognize the derivatized BSA carrier. The enriched binding population will undergo cycles of incubation with unconjugated BSA followed by Microcon® filtration. The non-specific oligonucleotides in the retentate will be discarded and those in the filtrate will be re-processed until base-line protein binding is obtained. Similar cycling will be performed by adding methanol extracts of negative soil samples prior to the addition of the target conjugate. Surrogate antibodies bound to the conjugate will be recovered for amplification. A final cycle of incubation using the unconjugated target congener, filtration, and amplification of SAb in the filtrate, will provide a polyclonal reagent free of derivative recognition. The consistent use of 10% MeOH in the selection buffers will enhance affinity and allow for higher PCB concentrations to be achieved in the final immunoassay. Published data on the use of MeOH indicates limited destabilization of a double helix relative to water (Albergo et al. (1981) Biochem 20:1413-8) suggesting that hydrophobic bonds are not a major component of duplex stability (Hickey et al. (1985) Biochem 9:2086-94)

Monoclonal surrogate antibodies will be produced from the enriched polyclonal reagent. Molecules having a single deoxyadenosine (A) at the 3′ end will be ligated using a pGEM-T EASY Vector® System (Promega). One sequence insert will ligate into each vector and produce individual bacterial colonies that have a single sequence. The presence of α-peptide in the vector sequence allows direct color screening of the recombinant clones on indicator plates. Clones containing the PCR fragments will produce white or light blue colonies. The PCR amplification and annealing protocols previously used will again be used to produce individual wells that contain monoclonal surrogate antibody. Each well will next be characterized.

Characterization and Method Development:

Black microplates, suitable for fluorescence detection, will be passively coated with the congener-BSA conjugate used for selection. Conjugates will be modified to alter the location or number of chlorine atoms if preferential conjugate binding of the SAb is observed. Standard validation protocols will be used to select molecules on the basis of affinity, congener cross-reactivity, cross-reactivity to related compounds or others that may be present, and matrix interferences. A database will be prepared to compare the performance of the SAbs and select one or more for use in the array. The performance advantage, if any, obtained by combining multiple monoclonal reagents into a polyclonal reagent for the test will be reviewed and considered. Selected surrogate antibody molecules will be sequenced and then synthesized to provide needed array-development material.

The characterization method will rely on detecting single, or double, FITC-labeled surrogate antibody molecules. The immunoassay protocol will incubate, in solution, surrogate antibody molecules with standards, samples, or controls. The reaction mixture will be added to microtiter plate wells coated with the target conjugate and blocked with 2% BSA. After 15-30 minutes the contents will be removed and the wells washed with a buffer containing Tween® 20. The signal will be quantified using a Wallac Victor II multi-label reader. Surrogate antibody titers will be quantified by testing doubling dilutions in 10% MeOH-Tris HCl buffer Dose-response characteristics will be calculated using an assay composed of a surrogate antibody dilution and 10 ppm congener illustrating 50% binding inhibition (B₅₀/ED₅₀). Dose-response curves will be produced using 5 congener standards. The curve will be linearized using a logit-log transform of the data to allow y=mx+b extrapolation of the data. The quantitation range of the competitive binding assay will typically extends from B₈₀ (i.e. 80% conjugate binding) to B₂₀ (20% Binding). The concentration range will span one to two logs depending upon the Ka of the surrogate antibody. The linearity of standard curves will be assessed from the correlation coefficient of the logit-log line (r²). Standard curves with a correlation coefficient ≧0.95, and % error of the duplicate standards ≦15%, will be used for calculating validation parameters (e.g. sensitivity, % cross-reactivity).

Preliminary % cross-reactivity will define the concentration of the non-target congeners needed to inhibit 50% of the surrogate antibody binding to the target congener. This ratio will be expressed as the % cross-reactivity. To develop an array having the characteristics shown in FIG. 13, surrogate antibody with <10% cross-reactivity will be selected. Similar studies will be performed using the compounds listed on the “specifications sheet” as possible cross-reactants. Spike-recovery studies using various sample matrices will evaluate relative matrix effects. Sensitivity, expressed as least detectable dose (LDD), minimum detection limit (MDL), practical quantitation limit (PQL) will be calculated as the extrapolated congener concentration equal to a multiple (e.g. LDD=2σ) of the signal standard deviation obtained from the simultaneous testing of multiple negative samples. Aroclors® will be tested at concentrations ≦10 ppm to verify detection capability and consistency with the anticipated response profiles (FIG. 11).

Surrogate antibody reagents for detecting each of the congeners will be combined and used with a microtiter plate having the five conjugates immobilized in adjacent wells. Unconjugated BSA will be immobilized to separate wells and used as a control. The assay will be used to test Aroclor® standards and spiked matrices. Profile array data will be collected and peak height vs. Aroclor correlation studies performed and collected. A total PCB, as opposed to an Aroclor identification assay format, will be evaluated by immobilizing a mixture of the 5 congener conjugates to individual microtiter wells. Samples will be incubated with the mixture surrogate antibody reagents and added to the mixed conjugate wells and BSA control wells. Standard FDA and EPA validation protocols will be performed to assess preliminary sensitivity, cross-reactivity, matrix interferences, and % recovery characteristics.

### Example 8

### Methods for Making a Ligand-Binding Surrogate Antibody Reagent that Recognizes IgG

As outlined in Example 5, surrogate antibody (SAb) molecules were produced using self-assembling oligonucleotide strands (87 nt+48 nt) to form a dimeric molecule having a 40 nt random specificity domain sequence with adjacent constant nucleotide sequences. Cycles of ligand binding, PCR amplification, bound/free separation, and reassembly/reannealing were used to enrich the SAb population with molecules that would bind an IgG polypeptide. Methods for the selection are discussed in detail in Example 1.

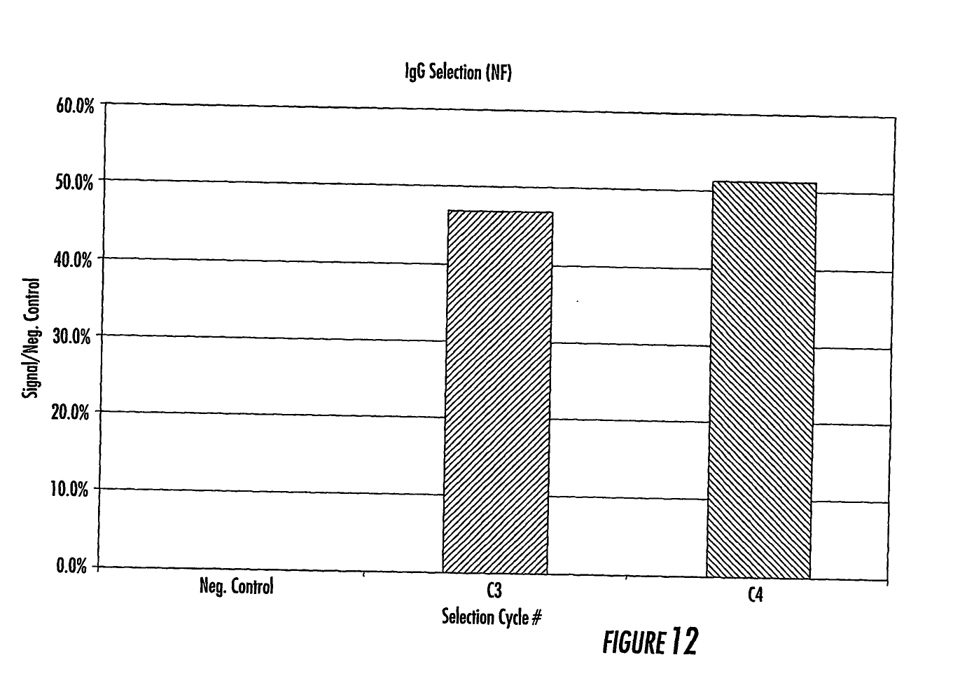

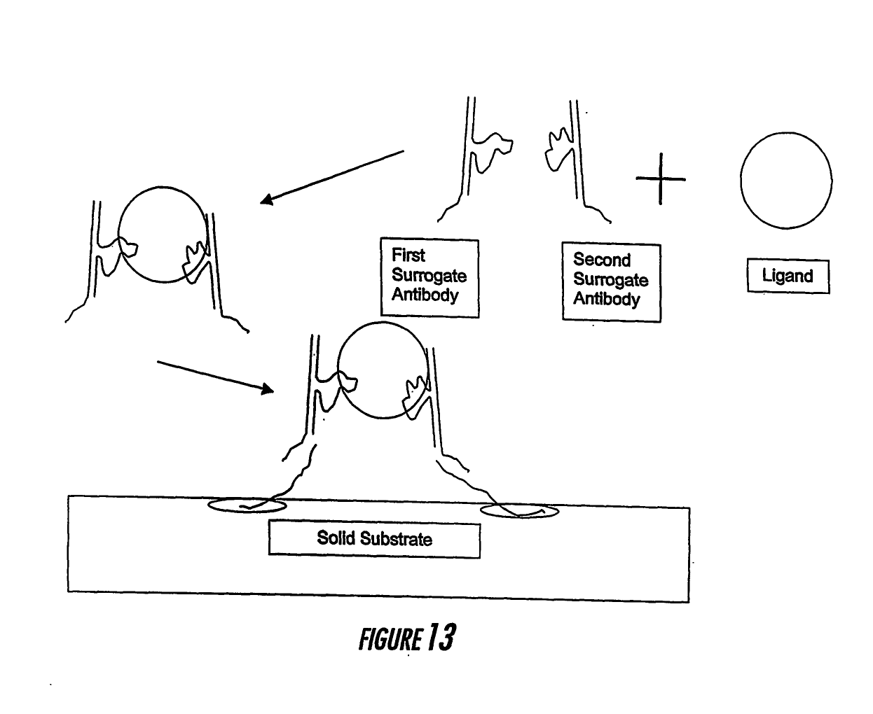

FIG. 12 illustrates the selection and enrichment of the surrogate antibodies to IgG. Signal/Negative control represents as a percent the amount of surrogate antibody bound to the target verses the amount of surrogate antibody recovered when the target is absent (negative control).

The following references are incorporated herein in their entirety for all purposes. References:
Ono et al. (1997) Nucleic Acids Research 25(22): 4581-4588
Peyman et al. (1996) Biol Chem Hoppe Seyler, 377(1): 67-70
Khan et al. (1997) J. Chrom. Biomed. Sci. Appl. 702(1-2):69-76
Maier et al. (1995) Biomed Pept Proteins Nucleic Acids 1(4):235-42
Boado et al. (1992) Bioconjug Chem 6:519-23
Jayasena et al. (1999) Clin Chem 45; 9:1628-1650
Dougan et al. (2000) Nucl Med Biol 27(3):289-97
Brody et al. (2000) J. Biotech.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

1. A method for detecting one or more ligands of interest in a population of test ligands, said method comprising:
a) contacting the population of test ligands with a population of surrogate antibody molecules under conditions that allow for the formation of a binding partner complex between at least one of the surrogate antibody molecules and at least one ligand of interest, wherein the surrogate antibody molecule comprises
i) a specificity strand having a specificity domain flanked by a first constant domain and a second constant domain;
ii) a stabilization strand comprising a first stabilization domain that interacts with said first constant domain and a second stabilization domain that interacts with said second constant domain; and,
iii) at least one oligonucleotide tail comprising a recognition nucleotide sequence that is unique to the particular surrogate antibody molecule;
b) forming at least one binding partner complex;
c) providing an array comprising a population of capture probes attached to a solid support, where the capture probes are attached to a discrete, known region of the solid support and comprise a capture nucleotide sequence that is complementary to at least one recognition nucleotide sequence;
d) contacting the binding partner complex with the array under conditions that allow for the hybridization of the recognition nucleotide sequence of the surrogate antibody with the capture nucleotide sequence of the corresponding capture probe; and
e) detecting the binding partner complex bound to the array to thereby detect the ligand of interest.

2. The method of claim 1, wherein the stabilization strand and the specificity strand are non-contiguous strands.

3. The method of claim 1, wherein the stabilization strand comprises an amino acid sequence.

4. The method of claim 1, wherein the stabilization strand comprises a nucleotide sequence.

5. The method of claim 1, wherein the recognition nucleotide sequence is about 4 to about 100 nucleotides in length.

6. The method of claim 1, wherein the step of detecting the binding partner complex bound to the array comprises at least one method selected from the group consisting of:
a) detecting the signal from a fluorescent group attached to the surrogate antibody molecule;
b) detecting the signal from a fluorescent group attached to the ligand of interest;
c) detecting the signal from a luminescent group attached to the surrogate antibody molecule;
d) detecting the signal from a luminescent group attached to the ligand of interest;
e) detecting the signal from a chromogenic group attached to the surrogate antibody molecule;
f) detecting the signal from a chromogenic group attached to the ligand of interest;
g) detecting a change in a fluorescent signal, where the change in the fluorescent signal results from the physical proximity of a fluorescent group found on the surrogate antibody molecule and a fluorescence modifying group found on the ligand of interest;
h) detecting a change in a fluorescent signal, where the change in the fluorescent signal results from the physical proximity of a fluorescent group found on the ligand of interest and a fluorescence modifying group found on the surrogate antibody molecule;
i) contacting the binding partner complex with a secondary molecule, where the secondary molecule contains a detectable label and binds specifically to the surrogate antibody molecule;
j) contacting the binding partner complex with a secondary molecule, where the secondary molecule contains a detectable label and binds specifically to the ligand of interest;
k) detecting the presence of a radioactive labeling group attached to the surrogate antibody molecule;
l) detecting the presence of a radioactive labeling group attached to the ligand of interest;
m) detecting the presence of an enzymatic labeling group attached to the surrogate antibody molecule;
n) detecting the presence of an enzymatic labeling group attached to the ligand of interest;
o) detecting a change in refractive index caused by the hybridization of the binding partner complex to the capture probe on the array;
p) detecting a change in electrical conductance caused by the hybridization of the binding partner complex to the capture probe on the array;
q) detecting a change in potential caused by the hybridization of the binding partner complex to the capture probe on the array; and
r) detecting a change in resistivity caused by the hybridization of the binding partner complex to the capture probe on the array.

7. The method of claim 6, wherein the step of detecting the binding partner complex bound to the array comprises at least one method selected from the group consisting of:
a) contacting the binding partner complex with a secondary molecule, wherein the secondary molecule is a second surrogate antibody molecule that contains a detectable label and binds specifically to the surrogate antibody molecule in the binding partner complex; and
b) contacting the binding partner complex with a secondary molecule, wherein the secondary molecule is a second surrogate antibody that contains a detectable label and binds specifically to the ligand of interest.

8. The method of claim 6, wherein the binding partner complex comprises at least two different surrogate antibody molecules bound to distinct epitopes on the ligand of interest and the step of detecting the binding partner complex bound to the array comprises at least one method selected from the group consisting of:
a) detecting a change in electrical conductance caused by the hybridization of the binding partner complex to the capture probe;
b) detecting a change in potential caused by the hybridization of the binding partner complex to the capture probe; and
c) detecting a change in resistivity caused by the hybridization of the binding partner complex to the capture probe.

9. The method of claim 1, wherein said step of detecting the binding partner complex bound to the array is performed in the presence of unbound test ligand and unbound surrogate antibody molecules.

10. The method of claim 1, wherein the population of test ligands is selected from the group consisting of:
a) a cell extract;
b) a tissue lysate
c) a clinical sample;
d) a water sample;
e) an industrial sample;
f) a food sample; and
g) a pharmaceutical sample.

11. The method of claim 1, wherein the ligand of interest is selected from the group consisting of:
a) a hapten;
b) a non-natural environmental chemical or biological agent;
c) a pathogen;
d) a carbohydrate;
e) a glycoprotein;
f) a muccopolysaccharide;
g) an enzyme;
h) a bacterium or molecule derived from a bacterium;
i) a virus or a molecule derived from a virus;
j) a protist or a molecule derived from a virus
k) an agent used in biological or chemical warfare;
l) a substance of abuse;
m) a therapeutic drug;
n) a hormone;
o) a peptide;
p) a polypeptide;
q) a prion; and
r) a molecule comprising a nucleic acid.

12. A method for detecting a ligand of interest in a population of test ligands comprising:
a) providing an array having a population of capture probes, where the capture probes are attached to discrete, known locations on a solid support, the capture probes comprise a known capture nucleotide sequence, and a population of surrogate antibody molecules are bound to the capture probes by an interaction between the capture nucleotide sequence and a recognition nucleotide sequence comprised within an oligonucleotide tail of the surrogate antibody, where the surrogate antibody molecules further comprise:
i) a specificity strand having a specificity domain flanked by a first constant domain and a second constant domain;
ii) a stabilization strand comprising a first stabilization domain that interacts with said first constant domain and a second stabilization domain that interacts with said second constant domain; and
iii) wherein the oligonucleotide trail comprises a recognition nucleotide is unique to the particular surrogate antibody molecule,
b) contacting a population of test ligands with the array under conditions that allow for the formation of a binding partner complex between at least one of the surrogate antibody molecules bound to the array and at least one of ligand of interest; and
c) detecting the binding partner complex.

13. The method of claim 12, wherein the stabilization strand and the specificity strand are non-contiguous strands.

14. The method of claim 12, wherein the stabilization strand comprises an amino acid sequence.

15. The method of claim 12, wherein the stabilization strand comprises a nucleotide sequence.

16. The method of claim 12, wherein the recognition nucleotide sequence is about 4 to about 100 nucleotides in length.

17. The method of claim 12, wherein the step of detecting the binding partner complex bound to the array comprises at least one method selected from the group consisting of:
a) detecting the signal from a fluorescent group attached to the surrogate antibody molecule;
b) detecting the signal from a fluorescent group attached to the ligand of interest;
c) detecting the signal from a luminescent group attached to the surrogate antibody molecule;
d) detecting the signal from a luminescent group attached to the ligand of interest;
e) detecting the signal from a chromogenic group attached to the surrogate antibody molecule;
f) detecting the signal from a chromogenic group attached to the ligand of interest;
g) detecting a change in a fluorescent signal, where the change in the fluorescent signal results from the physical proximity of a fluorescent group found on the surrogate antibody molecule and a fluorescence modifying group found on the ligand of interest;
h) detecting a change in a fluorescent signal, where the change in the fluorescent signal results from the physical proximity of a fluorescent group found on the ligand of interest and a fluorescence modifying group found on the surrogate antibody molecule;
i) contacting the binding partner complex with a secondary molecule, where the secondary molecule contains a detectable label and binds specifically to the surrogate antibody molecule;
j) contacting the binding partner complex with a secondary molecule, where the secondary molecule contains a detectable label and binds specifically to the ligand of interest;
k) detecting the presence of a radioactive labeling group attached to the surrogate antibody molecule;
l) detecting the presence of a radioactive labeling group attached to the ligand of interest;
m) detecting the presence of an enzymatic labeling group attached to the surrogate antibody molecule;
n) detecting the presence of an enzymatic labeling group attached to the ligand of interest;
o) detecting a change in refractive index caused by the hybridization of the binding partner complex to the capture probe on the array;
p) detecting a change in electrical conductance caused by the hybridization of the binding partner complex to the capture probe on the array;
q) detecting a change in potential caused by the hybridization of the binding partner complex to the capture probe on the array; and
r) detecting a change in resistivity caused by the hybridization of the binding partner complex to the capture probe on the array.

18. The method of claim 17, wherein the step of detecting the binding partner complex bound to the array comprises at least one method selected from the group consisting of:
a) contacting the binding partner complex with a secondary molecule, where the secondary molecule is a second surrogate antibody molecule that contains a detectable label and binds specifically to a surrogate antibody molecule in the binding partner complex; and
b) contacting the binding partner complex with a secondary molecule, where the secondary molecule is a second surrogate antibody that contains a detectable label and binds specifically to the ligand of interest.

19. The method of claim 12, wherein said step of detecting the binding partner complex bound to the array is performed in the presence of unbound test ligand and unbound surrogate antibody molecules.

20. The method of claim 12, wherein the population of test ligand is selected from the group consisting of:
a) a cell extract;
b) a tissue lysate
c) a clinical sample;
d) a water sample;
e) an industrial sample;
f) a food sample; and
g) a pharmaceutical sample.

21. The method of claim 12, wherein the ligand of interest is selected from the group consisting of:
a) a hapten;
b) an environmental toxin;
c) a pathogen;
d) a carbohydrate;
e) a glycoprotein;
f) a muccopolysaccharide;
g) an enzyme;
h) a bacterium or molecule derived from a bacterium;
i) a virus or a molecule derived from a virus;
j) a protist or a molecule derived from a virus k) an agent used in biological or chemical warfare;
l) a substance of abuse;
m) a therapeutic drug;
n) a hormone;
o) a peptide;
p) a polypeptide;
q) a prion; and
r) a molecule comprising a nucleic acid.

22. A method of producing an array comprising:
a) providing a solid support;
b) attaching to the solid support a population of capture probes, where the capture probes are attached to a discrete known region of the solid support and comprise a known capture nucleotide sequence;
c) providing a population of surrogate antibody molecules; wherein said surrogate antibody molecules comprise:
i) a specificity strand having a specificity domain flanked by a first constant domain and a second constant domain;
ii) a stabilization strand comprising a first stabilization domain that interacts with said first constant domain and a second stabilization domain that interacts with said second constant domain; and,
iii) at least one oligonucleotide tail comprising a recognition nucleotide sequence that is unique to the particular surrogate antibody molecule and binds to a capture nucleotide sequence;
d) contacting the solid support with the population of surrogate antibodies under conditions that allow for the hybridization of at least one capture nucleotide sequence with the corresponding recognition nucleotide sequence.

23. An array comprising:
a) a population of capture probes, where the capture probes are attached to discrete, known locations on a solid support and comprise a known capture nucleotide sequence; and
b) a population a surrogate antibody molecules that are bound to the capture probes by means of an interaction between the capture nucleotide sequence and a recognition nucleotide sequence comprised within an oligonucleotide tail of the surrogate antibody, wherein the surrogate antibody molecules further comprise:
i) a specificity strand having a specificity domain flanked by a first constant domain and a second constant domain; and
ii) a stabilization strand comprising a first stabilization domain that interacts with said first constant domain and a second stabilization domain that interacts with said second constant domain.

24. A kit comprising the array of claim 23.

25. A kit comprising:
a) a population of surrogate antibody molecules; wherein said surrogate antibody molecules comprise:
i) a specificity strand having at least one specificity domain flanked by a first constant domain and a second constant domain;
ii) a stabilization strand comprising a first stabilization domain that interacts with said first constant domain and a second stabilization domain that interacts with said second constant domain; and,
iii) at least one oligonucleotide tail comprising a recognition nucleotide sequence that is unique to the particular surrogate antibody molecule and binds to a capture nucleotide sequence;
b) an array comprising a solid support with a population of capture probes affixed thereto, where the capture probes are attached to a discrete, known region of a solid support, the capture probes comprise known capture nucleotide sequences, where the capture nucleotide sequence is complementary to and capable of hybridizing with a recognition sequence comprised in a surrogate antibody molecule.

26. The kit of claim 25, wherein the population of surrogate antibody molecules in the kit is lyophilized.

27. The kit of claim 25, wherein the kit further comprises a population of secondary molecules, where the secondary molecules comprise a detectable label and bind specifically to a ligand of interest.

28. The kit of claim 25, wherein the kit further comprises a population of secondary molecules, where the secondary molecules comprise a detectable label and bind specifically to one or more surrogate antibody molecules.

29. The kit of claim 27, wherein the population of secondary molecules is lyophilized.

30. The kit of claim 27, where the population of secondary molecules are surrogate antibody molecules.

31. A method for generating a ligand profile for a sample, said method comprising the steps of:
a) contacting the sample with a population of surrogate antibody molecules under conditions that allow for the formation of a binding partner complex between at least one of the surrogate antibody molecules and at least one ligand of interest in the sample, wherein the surrogate antibody molecule comprises
i) a specificity strand having a specificity domain flanked by a first constant domain and a second constant domain;
ii) a stabilization strand comprising a first stabilization domain that interacts with said first constant domain and a second stabilization domain that interacts with said second constant domain; and,
iii) at least one oligonucleotide tail comprising a recognition nucleotide sequence that is unique to the particular surrogate antibody molecule;
b) providing an array comprising a population of capture probes attached to a solid support, where the capture probes are attached to a discrete, known region of the solid support and comprise a capture nucleotide sequence that is complementary to at least one recognition nucleotide sequence;
c) contacting any binding partner complexes formed in step a) with the array under conditions that allow for the hybridization of the recognition nucleotide sequence of the surrogate antibody with the capture nucleotide sequence of the corresponding capture probe;
d) detecting the binding partner complex bound to the array; and
e) generating the ligand profile for the sample, wherein said ligand profile comprises values representing the level of one or more ligands that are present in the sample.

32. A method for generating a ligand profile for a sample, said method comprising the steps of:
a) providing an array having a population of capture probes, where the capture probes are attached to discrete, known locations on a solid support, the capture probes comprise a known capture nucleotide sequence, and a population of surrogate antibody molecules are bound to the capture probes by an interaction between the capture nucleotide sequence and a recognition nucleotide sequence comprised within an oligonucleotide tail of the surrogate antibody, where the surrogate antibody molecules further comprise:
i) a specificity strand having a specificity domain flanked by a first constant domain and a second constant domain;
ii) a stabilization strand comprising a first stabilization domain that interacts with said first constant domain and a second stabilization domain that interacts with said second constant domain; and
iii) wherein the oligonucleotide trail comprises a recognition nucleotide is unique to the particular surrogate antibody molecule,
b) contacting the sample with the array under conditions that allow for the formation of a binding partner complex between at least one of the surrogate antibody molecules bound to the array and at least one ligand of interest in the sample;
c) detecting the binding partner complex; and
d) generating the ligand profile for the sample, wherein said ligand profile comprises values representing the level of one or more ligands that are present in the sample.

33. A method for identifying a test sample, said method comprising:
a) providing one or more reference profiles, wherein each reference profile is characteristic of a particular type of sample and comprises values representing the levels of at least one ligand of interest in the sample;
b) providing a ligand profile for the test sample, wherein said ligand profile is generated according to the method of claim 31 or claim 32 and comprises values representing the level of one or more ligands of interest for which values are also comprised within the reference profiles; and
c) determining whether the ligand profile from the test sample is similar to one or more reference profiles to thereby identify the test sample.

34. A method for screening two or more samples to identify at least one ligand that is present at different levels in the samples, the method comprising
a) separately contacting each sample with a population of surrogate antibody molecules, wherein the surrogate antibody molecules comprise:
i) a specificity strand having a specificity domain flanked by a first constant domain and a second constant domain;
ii) a stabilization strand comprising a first stabilization domain that interacts with said first constant domain and a second stabilization domain that interacts with said second constant domain; and,
iii) at least one oligonucleotide tail comprising a recognition nucleotide sequence that is unique to the particular surrogate antibody molecule;
b) for each sample, forming one or more binding partner complexes between a surrogate antibody and a ligand if the sample contains a ligand that is bound by one or more surrogate antibodies in the population of antibodies;
c) for each sample, providing an array comprising a population of capture probes attached to a solid support, where the capture probes are attached to a discrete, known locations on the solid support and comprise a capture nucleotide sequence that is complementary to at least one recognition nucleotide sequence;
d) for each sample, contacting any binding partner complex formed in step b) with the array under conditions that allow for the hybridization of the recognition nucleotide sequence of the surrogate antibody with the capture nucleotide sequence of the corresponding capture probe;
e) for each sample, detecting any binding partner complex bound to the array; and
f) comparing the levels of the binding partner complex detected in each sample to thereby identify one or more ligands that are present at different levels in the samples.

35. The method of claim 33, wherein said method comprises the additional step of generating a ligand profile for one or more of the samples, wherein said ligand profile comprises values representing the level of one or more ligands that are present at different levels in the samples being compared.

36. A method for identifying a test sample, said method comprising:
a) providing a ligand profile for the test sample, wherein said ligand profile is generated according to the method of claim 33;
b) providing one or more reference profiles, wherein each reference profile is characteristic of a particular type of sample, and wherein the ligand profile for the test sample and each reference profile comprise one or more values representing the level of a ligand that is present at different levels in the populations of test ligands being compared; and
c) selecting the reference profile that is most similar to the ligand profile for the test sample to thereby identify the test sample.

37. A kit for identifying one or more samples, said kit comprising:
a) an array according to claim 23; and
b) a computer-readable medium having one or more digitally-encoded reference profiles wherein each reference profile of the plurality has a plurality of values, each value representing the level of a ligand detected by the array.

38. The kit of claim 25, wherein said kit additionally comprises a computer-readable medium having one or more digitally-encoded reference profiles wherein each reference profile of the plurality has a plurality of values, each value representing the level of a ligand detected by the array.
